SEQUENCE LISTING (48 sequences)

SEQ ID NO: 1 - RNA, 23 nt, Mus musculus
aggcaguguaauuagcugauugu

SEQ ID NO: 2 - RNA, 22 nt, Mus musculus
aaucacuaacuccacugccauc

SEQ ID NO: 3 - RNA, 23 nt, Homo sapiens
uaggcagugucauuagcugauug

SEQ ID NO: 4 - RNA, 22 nt, Homo sapiens
caaucacuaacuccacugccau

SEQ ID NO: 5 - DNA, 474 bp, Mus musculus
gcggccgctccgagggttacttgcacttagacctcgtgctcccggccctttgctgacgcatcctggctccggcctcggctttctgcggagtcagtggggctgcagcgctggcttctcctcccgcgggcggcgggtgatgctgtgccttgttttgatggcagtggagttagtgattgtcagcaccgcactacaatcagctaattacactgcctacaaaccgagcaccgggcgcccgccactgcagctcccgagggtcgggcccctcgccccctttcgccacggtcgacaggcgagggcggcggagcgagaggtgcctcaggctcccgaggcccctccacacccagcagggccgcgcgcgaccccaggtgaacccccaggcgctgaggccccctgtccccgccgtcccccccgagacccccgactcagcccggaccccagggcatccggcccgagtccttcttcccgcaaggatcc

SEQ ID NO: 6 - DNA, 84 bp, Homo sapiens
gtgctcggtttgtaggcagtgtcattagctgattgtactgtggtggttacaatcactaactccactgccatcaaaacaaggcac

SEQ ID NO: 7 - RNA, 84 nt, Homo sapiens
gugcucgguuuguaggcagugucauuagcugauuguacuguggugguuacaaucacuaacuccacugccaucaaaacaaggcac

SEQ ID NO: 8 - RNA, 84 nt, Mus musculus
gugcucgguuuguaggcaguguaauuagcugauuguagugcggugcugacaaucacuaacuccacugccaucaaaacaaggcac

SEQ ID NO: 9 - RNA, 23 nt, Mus musculus
aggcaguguaguuagcugauugc

SEQ ID NO: 10 - RNA, 22 nt, Mus musculus
aaucacuaaccacacagccagg

SEQ ID NO: 11 - RNA, 23 nt, Homo sapiens
aggcaguguaguuagcugauugc

SEQ ID NO: 12 - RNA, 22 nt, Homo sapiens
aaucacuaaccacacggccagg

SEQ ID NO: 13 - DNA, 480 bp, Mus musculus
gcggccgcagtcaatataatgaccaaatcagctaagggataatttctatttttccaatatatctaaaaatcacaaaaaatgtaccccacacaaattgatacattgtatacttagcagctaagggctagcggttccccccccccccccaaaccactaatagtatggtaagaatatttccctatggctctgtcctcaccaaaatgacgattcacaggaggctcagtcggaggaatttcagtctttttacctggctgtgtggttagtgattggtactattagcaatcagctaactacactgcctagtaactagactcagaaaaaagcatgcagtctttagctggtgctctcagactttggtgtgaccagagcaaatcgtcagccaagctgtggttgactctagtcgctgccttggtgatagctttctcagaagtggaaatcaggcagtgaatcacagcagcagcaggaactgttctgggatcc

SEQ ID NO: 14 - DNA, 77 bp, Homo sapiens
agtctagttactaggcagtgtagttagctgattgctaatagtaccaatcactaaccacacggccaggtaaaaagatt

SEQ ID NO: 15 - RNA, 77 nt, Homo sapiens
agucuaguuacuaggcaguguaguuagcugauugcuaauaguaccaaucacuaaccacacggccagguaaaaagauu

SEQ ID NO: 16 - RNA, 77 nt, Mus musculus
agucuaguuacuaggcaguguaguuagcugauugcuaauaguaccaaucacuaaccacacagccagguaaaaagacu

SEQ ID NO: 17 - RNA, 1162 nt, Homo sapiens
agcggaggccaaaucaacagcaacccuaagaacaagcauucuuuuuuuuuuuucaacagaacuaggccacacauauuuuuugucguuauuuaaauuuuuaguuguacuacauagaaaauaaacucauuuuaaaaaguuauuucaguaggcaaugcaucuucaugacuuuuacauaugaguuuuauuuuuuauuacuuuugaagaaaagucuguagaaaccuacuuuucaaggcaucugacccaaccauugccuuggauggcagcaauccagcucaggcacagcaucaccgccgcccggccgggaagaagacgccggcucggguagcccgcagccuucgagagaagaugccugagaagcgcggcgucggcguggguccugcgcagccugccccgcgagcgcccgcugcaagugcgaggaaacccgcgguuucuccagauacaguuaaacuguuagcucucucuaggagucacagaagaugaaacagucucaugccaggaaagcaaaaucccuggaggugaagccccuccauccauguaacaguuaauacuguaugcugugauucacugugucuauuugccaucgucuaguagaguauucaccaagcuagcaacucaguugagcuccaacucaaccaaugaauugccugccugucacaacguguugggguaccaacuugagacugcaauuuuuucuaugagucuaguuacuaggcaguguaguuagcugauugcuaauaguaccaaucacuaaccacacggccagguaaaaagauuugggaauucguccaaaugagcugccugugcaucaucaaugugcguggggaagagggguguuggaaaaugcugauuucauccauugccuauuaauugcucagccaaaagaaaaaaaucaacauuucagcuacuaaguuuacaauguauguaauguguauguaugugggguuuuguuuuguuuuguuuucaauauuccuucaggcucuuaaccaaaauuuuagauauaagggggaauaugauuuuuuucuuagcugacugauguauguuauuauaugaacaugugauuauuaacuucuugagacuauauuguuaguaauauuuugaaaguaauauuguuaguaauauuucgaaagaauaaagugccauaaagacaaaaaaaaaaaaaaaa

SEQ ID NO: 18 - DNA, 1162 bp, Homo sapiens
agcggaggccaaatcaacagcaaccctaagaacaagcattcttttttttttttcaacagaactaggccacacatattttttgtcgttatttaaatttttagttgtactacatagaaaataaactcattttaaaaagttatttcagtaggcaatgcatcttcatgacttttacatatgagttttattttttattacttttgaagaaaagtctgtagaaacctacttttcaaggcatctgacccaaccattgccttggatggcagcaatccagctcaggcacagcatcaccgccgcccggccgggaagaagacgccggctcgggtagcccgcagccttcgagagaagatgcctgagaagcgcggcgtcggcgtgggtcctgcgcagcctgccccgcgagcgcccgctgcaagtgcgaggaaacccgcggtttctccagatacagttaaactgttagctctctctaggagtcacagaagatgaaacagtctcatgccaggaaagcaaaatccctggaggtgaagcccctccatccatgtaacagttaatactgtatgctgtgattcactgtgtctatttgccatcgtctagtagagtattcaccaagctagcaactcagttgagctccaactcaaccaatgaattgcctgcctgtcacaacgtgttggggtaccaacttgagactgcaattttttctatgagtctagttactaggcagtgtagttagctgattgctaatagtaccaatcactaaccacacggccaggtaaaaagatttgggaattcgtccaaatgagctgcctgtgcatcatcaatgtgcgtggggaagaggggtgttggaaaatgctgatttcatccattgcctattaattgctcagccaaaagaaaaaaatcaacatttcagctactaagtttacaatgtatgtaatgtgtatgtatgtggggttttgttttgttttgttttcaatattccttcaggctcttaaccaaaattttagatataagggggaatatgatttttttcttagctgactgatgtatgttattatatgaacatgtgattattaacttcttgagactatattgttagtaatattttgaaagtaatattgttagtaatatttcgaaagaataaagtgccataaagacaaaaaaaaaaaaaaaa

SEQ ID NO: 19 - DNA, 102 bp, Mus musculus
ccagctgtgagtaattctttggcagtgtcttagctggttgttgtgagtattagctaaggaagcaatcagcaagtatactgccctagaagtgctgcacattgt

SEQ ID NO: 20 - RNA, 102 nt, Mus musculus
ccagcugugaguaauucuuuggcagugucuuagcugguuguugugaguauuagcuaaggaagcaaucagcaaguauacugcccuagaagugcugcacauugu

SEQ ID NO: 21 - RNA, 22 nt, Mus musculus
uggcagugucuuagcugguugu

SEQ ID NO: 22 - DNA, 110 bp, Homo sapiens
ggccagctgtgagtgtttctttggcagtgtcttagctggttgttgtgagcaatagtaaggaagcaatcagcaagtatactgccctagaagtgctgcacgttgtggggccc

SEQ ID NO: 23 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 24 - RNA, 22 nt, Homo sapiens
uggcagugucuuagcugguugu

SEQ ID NO: 25 - DNA, 20 bp, Artificial Sequence
  synthetic primer
cagaattgccattgcacaac

SEQ ID NO: 26 - DNA, 20 bp, Artificial Sequence
  synthetic primer
cagaagttggcatggtagcc

SEQ ID NO: 27 - DNA, 23 bp, Artificial Sequence
  synthetic primer
tcttgagcttggtgacaaaaact

SEQ ID NO: 28 - DNA, 20 bp, Artificial Sequence
  synthetic primer
gccattgtggcagatacaga

SEQ ID NO: 29 - DNA, 20 bp, Artificial Sequence
  synthetic primer
cactgtgcacaccccacagc

SEQ ID NO: 30 - DNA, 17 bp, Artificial Sequence
  synthetic primer
ttgcggtggacgatgga

SEQ ID NO: 31 - DNA, 21 bp, Artificial Sequence
  synthetic primer
ggcttgtcacgaattttgaga

SEQ ID NO: 32 - DNA, 20 bp, Artificial Sequence
  synthetic primer
gtatgttcggcttcccattc

SEQ ID NO: 33 - DNA, 37 bp, Artificial Sequence
  synthetic primer
gctcgctagcctcgagctgacacgctccgggtatcat

SEQ ID NO: 34 - DNA, 41 bp, Artificial Sequence
  synthetic primer
atgcctgcaggtcgacaagtcatatataataaatcatttat

SEQ ID NO: 35 - DNA, 41 bp, Artificial Sequence
  synthetic primer
tagcctcgagtctagagaactgacatcactccattttgccc

SEQ ID NO: 36 - DNA, 38 bp, Artificial Sequence
  synthetic primer
atgcctgcaggtcgaccggttattcagtgagaagcacc

SEQ ID NO: 37 - DNA, 66 bp, Artificial Sequence
  synthetic primer
gttaaaaaaaatataaacaaaagccgtaatacagcttgtcatacacattttggcagtattctccaa

SEQ ID NO: 38 - DNA, 66 bp, Artificial Sequence
  synthetic primer
ttggagaatactgccaaaatgtgtatgacaagctgtattacggcttttgtttatattttttttaac

SEQ ID NO: 39 - DNA, 35 bp, Artificial Sequence
  synthetic primer
gggacagcttgtggaagcgttgctgctgggaggcc

SEQ ID NO: 40 - DNA, 35 bp, Artificial Sequence
  synthetic primer
ggcctcccagcagcaacgcttccacaagctgtccc

SEQ ID NO: 41 - DNA, 28 bp, Artificial Sequence
  synthetic primer
cgcggatccttgcgggaagaaggactcg

SEQ ID NO: 42 - DNA, 33 bp, Artificial Sequence
  synthetic primer
atttgcggccgctccgagggttacttgcactta

SEQ ID NO: 43 - DNA, 36 bp, Artificial Sequence
  synthetic primer
gcggccgcagtcaatataatgaccaaatcagctaag

SEQ ID NO: 44 - DNA, 26 bp, Artificial Sequence
  primer
ggatcccagaacagttcctgctgctg

SEQ ID NO: 45 - DNA, 34 bp, Artificial Sequence
  synthetic primer
gctcgctagcctcgagactccctccatcccaacc

SEQ ID NO: 46 - DNA, 39 bp, Artificial Sequence
  synthetic primer
atgcctgcaggtcgacaagcttaaaaaggagtaggcggg

SEQ ID NO: 47 - DNA, 33 bp, Artificial Sequence
  synthetic primer
cccgccccccggtagctgccccggtgacacatc

SEQ ID NO: 48 - DNA, 33 bp, Artificial Sequence
  synthetic primer
gatgtgtcaccggggcagctaccggggggcggg

(19) United States
(12) Patent Application Publication
(10) Pub. No.: US 2024/0226134 A9
(43) Pub. Date: Jul. 11, 2024

(54) USE OF MICRORNAS IN THE TREATMENT OF FIBROSIS

(71) Applicant: FONDAZIONE TELETHON ETS, ROMA (RM) (IT)

(72) Inventors: Nicola BRUNETTI PIERRI, Roma (IT); Pasquale PICCOLO, Roma (IT); Rosa FERRIERO, Roma (IT)

(73) Assignee: FONDAZIONE TELETHON ETS, ROMA (RM) (IT)

(21) Appl. No.: 18/547,461

(22) PCT Filed: Feb. 28, 2022
(86) PCT No.: PCT/EP2022/055005

§ 371 Date: Aug. 22, 2023

(30) Foreign Application Priority Data
Mar. 1, 2021 (IT) 102021000004763

(51) Int. Cl. A61K 31/713; A61P 1/16; C12N 15/113; C12Q 1/6883

(52) U.S. Cl. CPC A61K 31/713; A61P 1/16; C12N 15/113; C12Q 1/6883; C12N 2310/141; C12Q 2600/178

(57) ABSTRACT

The present invention includes methods of treatment and/or prevention of fibrosis and of diseases associated with fibrosis by administering an, agent being selected from among: a combination of: miR-34b or a precursor or a mimic or a functional derivative thereof and miR-34c or a precursor or a mimic or a functional derivative thereof; or miR-34b or a precursor or a mimic or a functional derivative thereof or miR-34c or a precursor or a mimic or a functional derivative thereof.

### FIELD OF THE INVENTION

The present invention relates to at least one agent selected from miR-34b or miR-34c or a precursor or a mimic or a functional derivative thereof or a combination thereof for use in the treatment and/or prevention of fibrosis, in particular liver fibrosis, relative pharmaceutical compositions, nucleic acids, vectors and host cells.

### BACKGROUND OF THE INVENTION

Liver fibrosis is the deposition of scar tissue in the liver as consequence of chronic liver injury induced by multiple causes. Liver fibrosis can progress to cirrhosis, a condition that alters the organ architecture with aberrant vasculature and regenerative nodules, that ultimately results in portal hypertension, organ failure and hepatocellular carcinoma. Cirrhosis is a leading cause of morbidity and mortality worldwide and is expected to further increase in the next years ¹. Treatments for liver fibrosis are largely supportive and liver transplantation is the only life-saving option in advanced cirrhosis.

MicroRNAs (miRNAs) are small single-stranded non-coding RNAs of ˜22 nt in length that are responsible for fine tuning of gene expression. To date several miRNAs have been associated to the regulation of different processes contributing to liver fibrosis, particularly to hepatic stellate cells (HSC) activation into myofibroblast, a key step in the pathogenesis of liver fibrosis. For example, miR-21 is upregulated in HSC and promotes liver fibrosis through silencing of small mothers against decapentaplegic homolog 7 (SMAD7) that increases transforming growth factor β (TGF-β)/SMAD pro-fibrogenic signaling ^{2,3}. However, miR-21 pro-fibrotic role has been recently challenged, because miR-21 knock-out or knock-down did not affect liver fibrosis in mouse models ⁴. Conversely, HSC-enriched miR-29a is down-regulated in multiple models of liver fibrosis and exerts an antifibrotic activity by repressing collagen synthesis ^{5,6}. MiRNAs can also be released through extracellular vesicles to act as paracrine or endocrine effectors on other liver cells. Secretion of extracellular vesicles is enhanced by liver damage. Another miRNA secreted by HSC, the miR-214 can suppress connective tissue growth factor (CTGF)-mediated fibrogenesis in both HSC and hepatocytes ^{7,8}, while neutrophils can transfer miR-223 to hepatocytes and Kupffer cells promoting fibrosis resolution ^{9,10}. α1-antitrypsin (AAT) deficiency is one of the most common genetic diseases, it is an inherited disorder that affects ˜1 in 3,000 individuals and is an important genetic cause of lung and liver disease ¹¹. The most common defect is the Z variant of the SERPINA1 gene which results in the production of misfolded and polymerogenic Z α1-antitrypsin (ATZ). ATZ-dependent liver disease has a wide spectrum of clinical manifestations ranging from liver insufficiency in newborns to chronic liver disease and hepatocellular carcinoma in adults ^{12,13}. Because of its misfolding and polymerization, ATZ is unable to efficiently traverse the secretory pathway. Accumulation of ATZ in the endoplasmic reticulum (ER) of hepatocytes has a proteotoxic effect. Homozygous and heterozygous carriers of the Z allele of α1-antitrypsin are susceptible to develop liver fibrosis and cirrhosis. Fibrosis is a major health problem and unravelling its underlying pathogenic mechanisms has potential for the development of target therapeutic agents. It is therefore still felt the need of therapeutic agents able to treat fibrosis.

### SUMMARY OF INVENTION

Expression of microRNAs (miRNA) is affected in several liver diseases with distinct profiles across diseases with different etiologies ¹⁴. Here, inventors investigated differentially expressed miRNAs in the liver of the PiZ mice, a transgenic animal model expressing the human ATZ ¹⁵. Inventors then confirmed the most relevant findings in liver samples from patients. Following the identification of an important miRNA involved in liver fibrosis, the upstream molecules affecting its expression, and its effector, inventors showed this newly identified pathway is involved in various murine models of liver fibrosis.

In particular, in mouse and human samples expressing the Z allele of α1-antitrypsin, inventors found both miR-34b and miR-34c are upregulated by activation of FOXO3 upon JNK phosphorylation on Ser⁵⁷⁴. Deletion of miR-34b and miR-34c results in early development of liver fibrosis and increased signaling of the PDGF pathway, a target of miR-34b and c. JNK-activated FOXO3 and miR-34b and miR-34c upregulation also occurs in several mouse models of liver fibrosis.

Then, inventors herein investigated the role of miR-34b and miR-34c in liver fibrosis and using multiple models they found miR-34b and miR-34c anti-fibrotic activity in mice and in a human cell culture system, showing that miR-34b and/or miR-34c may be used as antifibrotic drugs.

### DESCRIPTION OF THE INVENTION

Liver fibrosis is a major complication of chronic liver diseases and is orchestrated by a complex molecular network. MicroRNAs have been found to regulate several pathophysiological processes, including liver fibrosis. Mir-34 family is upregulated in response to several chronic liver insults and inventors have herein found miR-34b and/or miR-34c to silence the platelet derived growth factor signaling, thus protecting against liver fibrosis. The inventors further show a protective effect of miR-34b and/or miR-34c against liver fibrosis in various mouse models. MiR-34b and/or miR-34c were effective in blunting TGF-β-mediated activation of human hepatic stellate cells, a key event in liver fibrosis development thereby inhibiting activation of hepatic stellate cells, and directly inhibit collagen biosynthesis. Finally, inventors found that hepatocyte-specific delivery of mR-34b and/or miR-34c significantly ameliorated liver fibrosis in two independent mouse models of liver fibrosis. In conclusion, an antifibrotic activity was shown for miR-34b and/or miR-34c thus indicating a novel therapy against hepatic fibrogenesis.

The present inventors have found that miR-34b-5p and miR-34c-5p (herein defined as miR-34b and miR-34c) are upregulated mainly in hepatocytes and prevent fibrosis by inhibition of platelet-derived growth factor (PDGF) signaling in liver disease due to α-1 antitrypsin deficiency, a disorder prone to liver fibrosis. Moreover, inventors also found that miR-34b and miR-34c upregulation occurs in several other mouse models of liver fibrosis, suggesting that miR-34b and miR-34c have a broader involvement in fibrosis as an anti-fibrotic mechanism. α1-antitrypsin (AAT) deficiency is a common genetic disease presenting with lung and liver diseases. AAT deficiency results from pathogenic variants in the SERPINA1 gene encoding AAT and the common mutant Z allele of SERPINA1 encodes for ATZ, a protein forming hepatotoxic polymers retained in the endoplasmic reticulum of hepatocytes. PiZ mice express the human ATZ and are a valuable model to investigate the human liver disease of AAT deficiency. Inventors herein investigated differential expression of miRNAs between PiZ and control mice and inventors found that miR-34b and miR-34 c were upregulated, and their levels correlated with intrahepatic ATZ. Furthermore, in PiZ mouse livers, inventors found that FOXO3 driving miR-34b and c expression was activated and miR-34b and miR-34c expression was dependent upon JNK phosphorylation on Ser574. Deletion of miR-34b and/or miR-34c in PiZ mice resulted in early development of liver fibrosis and increased signaling of PDGF, a target of miR-34b and miR-34 c. Activation of FOXO3 and increased miR-34c were confirmed in livers of humans with AAT deficiency. In addition, JNK-activated FOXO3 and miR-34b and miR-34c upregulation were detected in several mouse models of liver fibrosis. The inventors thus revealed a novel pathway involved in liver fibrosis and potentially implicated in both genetic and acquired causes of hepatic fibrosis.

It is therefore an object of the invention at least one agent for use in the treatment and/or prevention of fibrosis and/or of diseases associated with fibrosis, said agent being selected from the group consisting of:
a combination of:
   (i) miR-34b or a precursor or a mimic or a functional derivative thereof and
   (ii) miR-34c or a precursor or a mimic or a functional derivative thereof; or
miR-34b or a precursor or a mimic or a functional derivative thereof or
miR-34c or a precursor or a mimic or a functional derivative thereof.

Any combination of two or more of the agents defined above is comprised in the present invention.

Preferably, said agent is a combination of miR-34b or a precursor or a mimic or a functional derivative thereof and miR-34c or a precursor or a mimic or a functional derivative thereof; or it is miR-34b or a precursor or a mimic or a functional derivative thereof.

Preferably, the agent comprises a double-stranded RNA molecule 22 to 24 base pairs in length comprising:
a) an active strand comprising miR-34b or miR-34c and
b) a passenger strand comprising a sequence that is at least 60%, 70%, 80%, 90% or 100% complementary to the active strand,
optionally said RNA molecule being blunt-ended.

Preferably, miR-34b comprises or consists of the SEQ ID NO: 3 or 1. Preferably, the miR-34c comprises or consists of the SEQ ID NO:11 or 9.

Preferably, the agent is provided within a delivery vehicle, optionally wherein the delivery vehicle is selected from a vector, preferably a recombinant expression vector or a viral vector, or a delivery vehicle selected from nanoparticles, microparticles, liposomes or other biological or synthetic vesicle or material including lipid nanoparticles, polymer-based nanoparticles, polymer-lipid hybrid nanoparticles, microparticles, microspheres, liposomes, colloidal gold particles, graphene composites, cholesterol conjugates, cyclodextran complexes, polyethylenimine polymers, lipopolysaccharides, polypeptides, polysaccharides, lipopolysaccharides, collagen, pegylation of viral vehicles.

Another object of the invention is a nucleic acid coding for the agent as defined herein for use in the treatment and/or prevention of fibrosis and/or of diseases associated with fibrosis.

A further object of the invention is a vector, preferably a recombinant expression vector, comprising a coding sequence for the agent as defined herein or the nucleic acid as defined above and/or expressing the agent as defined in herein, preferably under the control of a suitable promoter, for use in the treatment and/or prevention of fibrosis and/or of diseases associated with fibrosis. Preferably the vector is a viral or non-viral vector, preferably the viral vector is selected from adeno-associated virus (AAV) vectors, lentivirus vectors, adenoviral vector, retroviral vectors, alphaviral vectors, vaccinia virus vectors, herpes simplex virus (HSV) vectors, rabies virus vectors, and Sindbis virus vectors.

Another object of the invention is a host cell transformed with the vector as defined above for use in the treatment and/or prevention of a fibrosis and/or of diseases associated with fibrosis. A further object of the invention is a recombinant adeno-associated virus (rAAV) particle comprising a nucleic acid encoding miR-34b and/or miR-34c or a precursor or a mimic or a functional derivative thereof, preferably the particle comprises a capsid derived from adeno-associated vectors AAV8, AAV1, AAV2, AAV5, or AAV9, preferably wherein the nucleic acid is operably linked to an hepatocyte-specific thyroxine binding protein promoter, for use in the treatment of fibrosis and/or of diseases associated with fibrosis.

Another object of the invention is a pharmaceutical composition for use in the treatment of fibrosis and/or of diseases associated with fibrosis comprising an agent or the nucleic acid or the vector or the host cell according or a recombinant adeno-associated virus (rAAV) particle as defined herein and at least one pharmaceutically acceptable carrier and/or diluents.

A further object of the invention is a method for the diagnosis of fibrosis and/or of diseases associated with fibrosis and/or for determining the activity, the stage, or the severity of fibrosis in a subject, and/or for the classification of a subject as a receiver or non receiver of a treatment for fibrosis and/or for diseases associated with fibrosis, and/or for the evaluation of the efficacy of a medical treatment, and/or for the determination of the progression or the regression of the disease in fibrosis and/or in diseases associated with fibrosis patients, and/or for the classification of a patient as a potential responder or non responder to a medical treatment, and/or for the prediction of disease outcome for a patient comprising determining the level of miR-34b and/or miR34c in a sample obtained from a subject and comparing it with a proper control.

Another object of the invention is a kit for the diagnosis of fibrosis and/or of diseases associated with fibrosis and/or for determining the activity, the stage, or the severity of fibrosis and/or of diseases associated with fibrosis in a subject, and/or for the classification of a subject as a receiver or non receiver of a treatment for fibrosis and/or of diseases associated with fibrosis, and/or for the evaluation of the efficacy of a medical treatment, and/or for the determination of the progression or the regression of the disease in fibrosis and/or in diseases associated with fibrosis patients, and/or for the classification of a patient as a potential responder or non responder to a medical treatment, and/or for the prediction of disease outcome comprising primers and/or probes specific for miR-34b and miR-34c, or for miR-34b or for miR-34c, the kit preferably further comprising miRNA isolation and/or purification means.

Preferably the fibrosis is a fibrosis of liver, lungs, kidneys, skin, joints, even more preferably of liver or lungs.

Preferably the disease associated with fibrosis is an acquired or genetic diseases selected from the group consisting of: Cholestatic liver diseases, such as Primary Sclerosing Cholangitis, Primary Biliary Cholangitis, Primary Familiar Intrahepatic Cholestasis, Non-alcoholic fatty liver disease (NAFLD)/Non-alcoholic steatohepatitis (NASH) preferably with advanced fibrosis, Viral hepatitis, Genetic diseases affecting liver, such as Wilson disease, Primary Familiar Intrahepatic Cholestasis, AlAT deficiency, Haemochromatosis, Congenital Hepatic Fibrosis.

The fibrosis can be at any stage. In an embodiment the fibrosis is at an advanced stage.

### DETAILED DESCRIPTION OF THE INVENTION

MicroRNAs (miRNAs) are a class of non-coding RNAs that play important roles in regulating gene expression. The majority of miRNAs are transcribed from DNA sequences into primary miRNAs and processed into precursor miRNAs, and finally mature miRNAs. In most cases, miRNAs interact with the 3′ untranslated region (3′ UTR) of target mRNAs to induce mRNA degradation and translational repression. However, interaction of miRNAs with other regions, including the 5′ UTR, coding sequence, and gene promoters, have also been reported.

The seed sequence is essential for the binding of the miRNA to the mRNA. The seed sequence or seed region is a conserved heptametrical sequence which is mostly situated at positions 2-7 from the miRNA 5′-end. Besides seed match, additional sequence features were shown to affect miRNA-target recognition and silencing efficiency.

miRNAs are frequently complementary to the 3′ UTR of the mRNA transcript, however, miRNAs of the invention may bind any region of a target mRNA. Alternatively, or in addition, miRNAs target methylation genomic sites which correspond to genes encoding targeted mRNAs.

Mature miRNAs may have a length of about 19-24 nucleotides (and any range in between), particularly 21, 22 or 23 nucleotides. The miRNAs, however, may be also provided as a precursor which may have a length of about 70 to about 100 nucleotides (pre-miRNA). The precursor may be produced by processing of a primary transcript which may have a length of greater than about 100 nucleotides (pri-miRNA). The miRNA as such may usually be a single-stranded molecule, while the miRNA-precursor may be in the form of an at least partially self-complementary molecule capable of forming double-stranded portions, e.g. stem- and loop-structures. DNA molecules encoding the miRNA, pre-miRNA and pri-miRNA molecules are also encompassed by the invention. The nucleic acids may be selected from RNA, DNA or nucleic acid analog molecules, such as sugar- or backbone-modified ribonucleotides or deoxyribonucleotides. It should be noted, however, that other nucleic analogs, such as peptide nucleic acids (PNA) or locked nucleic acids (LNA), may also be suitable.

miRNAs of the invention include miRNA34b and/or miRNA34c and homologs, analogs and orthologues thereof, primary miRNA molecules, precursor miRNA molecules, mature miRNA molecules, and DNA molecules encoding said miRNAs.

In the context of the present invention the term “miR34b”, “miR-34b”, “miRNA-34b”, microRNA-34b″, “miR-34b-5p” are used interchangeably. In the context of the present invention the term “miR34c”, “miR-34c”, “miRNA-34c”, microRNA-34c″, “miR-34c-5p” are used interchangeably. Within the present invention, lower case letters are used to indicate both DNA and RNA molecules, including but not limited to genomic DNA and RNA transcripts. When used, upper case letters indicate genomic miRNA sequences.

Conveniently, the terms “miR34b” and “miR34c” encompass homologs, analogs and orthologues thereof, primary miRNA molecules, precursor miRNA molecules, mature miRNA molecules, and DNA molecules encoding said miRNAs. Such terms include miR-34b-5p, miR-34b-3p, miR-34c-5p and miR-34c-3p. Optionally, miR34b and/or miR-34c do not include miR-34b-3p, and/or miR-34c- 3p.

As used herein, miR34b/c means miR34b, miR34c and/or a combination of miR34b and miR34c.

MiRNAs of the present invention may be a combination of miR34b and miR34c, homologs and analogs thereof, wherein miR34b and miR34c may be primary miRNA molecules, precursor miRNA molecules, mature miRNA molecules, and DNA molecules encoding said miRNAs. Conveniently, said combinations of miR34b and miR34c may be encoded within a single nucleotide sequence or multiple nucleotide sequences, as a primary transcript or DNA encoding said primary transcript, as a polycistronic or bicistronic DNA molecule e.g. the two miRNA encoding DNA sequences are linked by a sequence that recruits ribosomes and allows cap-independent translation, for instance (but not limited to) IRES or E2A sequences.

As defined herein, the term “functional derivative” of a miRNA refers to a miRNA that has less than 100% identity to a corresponding wild-type miRNA and possesses one or more biological activities of the corresponding wild-type miRNA. Examples of such biological activities include, but are not limited to, inhibition of expression of a target RNA molecule (e g, inhibiting translation of a target mRNA molecule and/or modulating the stability of a target mRNA molecule) and inhibition of a cellular process associated therewith. These functional derivatives include species variants and variants that are the consequence of one or more mutations (e.g., a substitution, a deletion, an insertion) in a miRNA-encoding gene. In certain embodiments, the variant is at least about 87%, 90%, 95%, 98%, or 99% identical to a corresponding wild-type miRNA. Functional derivatives also encompass “functional fragments” of a miRNA, i.e., portions of miRNA which are less than the full-length molecule (and their species and mutant variants) and that possess one or more biological activities of a corresponding wild-type miRNA. In certain embodiments, the biologically-active fragment is at least about 7, 10, 12, 15, or 17 nucleotides in length. In other embodiments, the biologically active fragment is at least 7 or more nucleotides, preferably at least 8 or more nucleotides. The functional derivative may also include longer sequences or shifted sequences, compared to the miRNA; optionally, the functional derivative may include longer sequences or shifted sequences from said miRNA genomic sequence.

The term “functional derivative” also includes:
variants of mature miR-34b comprising sequences with at least 87% sequence identity to miR-34b mature sequence, optionally wherein the SEED sequence GGCAGUG is preserved (i.e. wherein nucleotide changes are not within the SEED sequence), or DNA molecules encoding said miRNAs,
variants of mature miR-34b comprising sequences with at least 86% sequence identity to miR-34b mature sequence, said percentage calculated in the sequence not comprising the seed, optionally wherein the SEED sequence GGCAGUG is preserved (i.e. wherein nucleotide, changes are not within the SEED sequence), or DNA molecules encoding said miRNAs
variants of mature miR-34c comprising sequences with at least 86% sequence identity to miR-34c mature sequence, optionally wherein the SEED sequence GGCAGUG is preserved (i.e. wherein nucleotide changes are not within the SEED sequence), or DNA molecules encoding said miRNAs,
variants of mature miR-34c comprising sequences with at least 81% sequence identity to miR-34c mature sequence, said percentage calculated in the sequence not comprising the seed, optionally wherein the SEED sequence GGCAGUG is preserved (i.e. wherein nucleotide changes are not within the SEED sequence), or DNA molecules encoding said miRNAs.

Also included in the present invention are variants of primary miRNA and precursor miRNAs of the invention comprising a sequence with at least 50%, 60%, 70%, 75%, 80%, 85%, 90%, 95% identity to the reference sequences or DNA molecules encoding said miRNAs.

Mature miRNAs of the present invention do not consist of the sequence of miR34a, i.e. of SEQ ID NO: 21 and 24.

As intended herein, a precursor may be a primary miRNA and/or a precursor miRNA.

The present invention encompasses agents capable of increasing the level, activity, function and/or efficacy of miR-34b and/or miR-34c. An agent within the meaning of the present invention may be a nucleic acid, a peptide or peptidomimetic, an antibody or antibody fragment, a small molecule, agonist, antagonist, aptamer. Preferred agents are miR34b and/or miR34c primary miRNA molecules, precursor miRNA molecules, mature miRNA molecules, miRNA mimetics or mixture thereof, DNA molecules encoding said primary miRNA molecules, precursor miRNA molecules, mature miRNA molecules, miRNA mimetics or mixture thereof. Preferably, the peptide is JNK1/2 (Gene ID: 51528 and 5601) and FOXO3 (Gene ID: 2309).

Agents of the present invention may be agonist, antagonist, aptamers, wherein agonists are intended to be molecules which directly increase levels, activity, function and/or efficacy of miRNAs of the invention; antagonists and aptamers are intended to be molecules which antagonize the activity of molecules or factors which lead to inactivation of miRNAs of the invention, indirectly resulting in increased levels, activity, function and/or efficacy of the miRNAs of the invention.

As used herein, the term “miRNA mimic” refers to a double-stranded miRNA-like RNA fragment. Such miRNA mimic is designed to have its 5′-end bearing a partially complementary motif to the selected sequence in the 3′UTR unique to the target mRNA. Once introduced into cells, miRNA mimic, mimicking an endogenous miRNA, can bind to its target mRNA and inhibit its translation and/or modulate its stability. Unlike endogenous miRNAs, miR-mimics can be made to act in a gene-specific fashion by increasing the region of perfect complementarity with mRNA 3′ UTR. Often, miRNA mimics are made to harbor chemical modifications to improve stability and/or cellular uptake (Rooij and Kauppinen, EMBO Mol Med., 2014, 6(7): 851-864, which is incorporated herein by reference in its entirety). In such double-stranded miRNA mimics, the strand identical to the miRNA of interest is the guide (antisense) strand, while the opposite (passenger or sense) strand is less stable and can be linked to a molecule, such as, e.g., cholesterol, to enhance cellular uptake. In addition, the passenger strand may contain chemical modifications to prevent RISC loading, while it is further left unmodified to ensure rapid degradation. Since the miRISC needs to recognize the guide strand as a miRNA, the chemical modifications that can be used for the guide strand are limited. For example, the 2¢-fluoro (2¢-F) modification helps to protect against exonucleases, hence making the guide strand more stable, while it does not interfere with RISC loading (Rooij and Kauppinen, EMBO

Mol Med., 2014, 6(7): 851-864, which is incorporated herein by reference in its entirety). Preferably, an additional treatment agent is administered with the agent as disclosed above.

Delivery vehicles within the meaning of the present disclosure may be vectors as defined herein or delivery systems or particles including but not limited to nanoparticles, microparticles or liposomes as defined herein.

The terms “vector”, “expression vector” and “expression construct”, “recombinant expression vector”, “recombinant expression construct”, “recombinant vector”, are used interchangeably to refer to a composition which can be used to deliver a nucleic acid of interest to the interior of a cell and mediate its expression within the cell. Most commonly used examples of vectors are autonomously replicating plasmids and viruses (such as, e.g., adenoviral vectors, adeno-associated virus vectors (AAV), lentiviral vectors, Sindbis virus vectors, etc.). An expression construct can be replicated in a living cell, or it can be made synthetically. In one embodiment, an expression vector comprises a promoter operably linked to a polynucleotide (e.g., a polynucleotide encoding miR-34b and/or miR-34c or its functional derivative or mimic) which promoter controls the initiation of transcription by RNA polymerase and expression of the polynucleotide. Typical promoters for mammalian cell expression include, e.g., SV40 early promoter, CMV immediate early promoter (see, e.g., U.S. Pat. Nos. 5,168,062 and 5,385,839, both of which are incorporated herein by reference in their entirety), mouse mammary tumor virus LTR promoter, adenovirus major late promoter (Ad MLP), herpes simplex virus promoter, murine metallothionein gene promoter, and U6 or H1 RNA pol III promoter. Non-limiting examples of promoters useful for expressing miR-34b and/or miR-34c in the methods of the present disclosure include liver-specific promoters, including but not limited to hepatocyte-specific promoters, for instance thyroxine binding protein promoter, lung-specific promoters for example surfactant protein B gene promoter, kidney specific promoters, for instance Kidney-specific cadherin promoter, , skin promoters, for instance Keratin 14 promoter targeting gene expression to keratinocytes of the epidermal basal layer, CD11c promoter targeting gene expression to dendritic cells, fascin promoter targeting gene expression to mature dendritic cells; joints specific promoters, Synapsin promoter (neuron specific), CamKIIa promoter (specific for excitatory neurons), ubiquitin promoter, CAG promoter, CMV promoter, and b-actin promoter. These and other promoters can be obtained from commercially available plasmids, using techniques well known in the art. See, e.g., Sambrook et al., supra. Enhancer elements may be used in association with promoters to increase expression levels of the vectors. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777, which is incorporated herein by reference in its entirety, and elements derived from human CMV, as described in Boshart et al., Cell (1985) 41:521, which is incorporated herein by reference in its entirety, such as elements included in the CMV intron A sequence.

Typically, transcription terminator/polyadenylation signals will also be present in the expression vector.

The recombinant expression vector of the invention can be any suitable recombinant expression vector, and can be used to transform or transfect any suitable host. Suitable vectors include those designed for propagation and expansion or for expression or both, such as plasmids and viruses. The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell.

Replication systems can be derived, e.g., from CoIEl, 2 μ plasmid, λ, S V40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA- based. The recombinant expression vector can include one or more marker genes, which allow for selection of transformed or transfected hosts.

The recombinant expression vector can comprise a native or normative promoter operably linked to the nucleotide sequence encoding the miR-34b, miR-34c, and/or mimics thereof (including functional portions and functional variants thereof), or to the nucleotide sequence which is complementary to or which hybridizes to the nucleotide sequence encoding the RNA.

The selection of promoters, e.g., strong, weak, inducible, tissue- specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an S V40 promoter, an RSV promoter, and a promoter found in the long-terminal repeat of the murine stem cell virus. Prefered promoter is the thyroxine binding protein promoter.

The recombinant expression vectors can be designed for either transient expression, for stable expression, or for both. Also, the recombinant expression vectors can be made for constitutive expression or for inducible expression.

The level of the miR is preferably determined using a method selected from hybridization, array-based assays, PCR-based assays, and sequencing, wherein the PCR-based assay is quantitative PCR (qPCR). The level of the miR is preferably determined prior to the administration of the treatment or both prior and after the administration of the treatment.

The nucleic acid molecules of the invention may be obtained by chemical synthesis methods or by recombinant methods, e.g. by enzymatic transcription from synthetic DNA-templates or from DNA-plasmids isolated from recombinant organisms. Typically phage RNA- polymerases are used for transcription, such as T7, T3 or SP6 RNA-polymerases.

An agent within the meaning of the invention may also comprise a recombinant expression vector comprising a recombinant nucleic acid operatively linked to an expression control sequence, wherein expression, i.e. transcription and optionally further processing results in a miRNA-molecule or miRNA precursor (pri- or pre-miRNA) molecule as described above. The vector may be an expression vector suitable for nucleic acid expression in eukaryotic, more particularly mammalian cells. The recombinant nucleic acid contained in said vector may be a sequence which results in the transcription of the miRNA-molecule as such, a precursor or a primary transcript thereof, which may be further processed to give the miRNA-molecule. A delivery system or vehicle may be a vector of viral or non-viral origin.

Alternative delivery systems or vehicles for the agents of the invention as defined above comprise nanoparticles, microparticles, liposomes or other biological or synthetic vesicle or material.

Additional non limiting examples of said delivery systems or vehicles include but are not limited to lipid nanoparticles, polymer-based nanoparticles, polymer-lipid hybrid nanoparticles, microparticles, microspheres, liposomes, colloidal gold particles, graphene composites, cholesterol conjugates, cyclodextran complexes, polyethylenimine polymers, lipopolysaccharides, polypeptides, polysaccharides, lipopolysaccharides, collagen, pegylation of viral vehicles.

In some aspects, the agent of the invention may be an RNA-or DNA molecule, which may contain at least one modified nucleotide analog, i.e. a naturally occurring ribonucleotide or deoxyribonucleotide is substituted by a non-naturally occurring nucleotide. The modified nucleotide analog may be located for example at the 5′-end and/or the 3′-end of the nucleic acid molecule.

Nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase, such as uridines or cytidines modified at the 5-position, e.g. 5-(2-amino)propyl uridine, 5- bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine may be suitable. In sugar-modified ribonucleotides the 2′-OH-group is replaced by a group selected from H, OR, R, halo, SH, SR, NH 2 , NHR, NR 2 or CN, wherein R is C 1-C 6 alkyl, alkenyl or alkynyl and halo is F, CI, Br or I. In preferred backbone-modified ribonucleotides the phosphoester group connecting to adjacent ribonucleotides is replaced by a modified group, e.g. of phosphothioate group. It should be noted that the above modifications may be combined.

In the present invention “miR mimics or mimetics” are small double-stranded RNA oligonucleotides, that can be chemically modified and that mimic endogenous miRNAs; the mimic or mimetic sequence comprises or corresponds to the sequence of the mature miRNA.

Mimics or mimetic of miR34b and/or miR34c may be produced by many techniques known in the art. The 2′ hydroxyl group of the ribose sugars may be alkylated, such as by methylation, to increase the stability of the molecule. Also, the ribose sugars may be modified by replacement of the hydroxyl group at the 2′ position with a hydrogen, thus generating a DNA backbone. Also, any uracil base of an RNA sequence may be replaced by thymine. These are only a few non-limiting examples of the possible modifications that may be performed by a skilled artisan. A miR and mimics thereof can be administered in a composition (e.g., pharmaceutical composition) that can comprise at least one excipient (e.g., a pharmaceutically acceptable excipient), as well as other therapeutic agents (e.g., other miRs and/or mimics thereof). The composition can be administered by any suitable route, including parenteral, topical, oral, or local administration.

The administration of oligonucleotides of the present invention may be carried out by known methods, wherein a nucleic acid is introduced into a desired target cell in vitro or in vivo. An aspect of the present invention comprises a nucleic acid construct comprised within a delivery vehicle. A delivery vehicle is an entity whereby a nucleotide sequence can be transported from at least one media to another. Delivery vehicles may be generally used for expression of the sequences encoded within the nucleic acid construct and/or for the intracellular delivery of the construct. It is within the scope of the present invention that the delivery vehicle may be a vehicle selected from the group of RNA based vehicles, DNA based vehicles/vectors, lipid based vehicles, virally based vehicles and cell based vehicles, protein-based vehicles, polymer- based vehicles. Examples of such delivery vehicles include: biodegradable polymer microspheres, lipid based formulations such as liposome carriers, coating the construct onto colloidal gold particles, lipopolysaccharides, polypeptides, polysaccharides, pegylation of viral vehicles.

In one embodiment of the present invention may comprise a virus as a delivery vehicle, where the virus may be selected from: adenoviruses, retroviruses, lentiviruses, adeno-associated viruses, herpesviruses, vaccinia viruses, foamy viruses, cytomegaloviruses, Semliki forest virus, poxviruses, RNA virus vector and DNA virus vector. Such viral vectors are well known in the art.

Commonly used gene transfer techniques include calcium phosphate, DEAE-dextran, transfection, electroporation and microinjection and viral methods. Another technique for the introduction of DNA into cells is the use of cationic liposomes. Commercially available cationic lipid formulations are e.g. Tfx 50 (Promega) or Lipofectamin 2000 (Life Technologies).

The compositions of the present invention may be in form of a solution, e.g. an injectable solution, a cream, ointment, tablet, suspension or the like. The composition may be administered in any suitable way, e.g. by injection, , by oral, topical, nasal, rectal application etc. The carrier may be any suitable pharmaceutical carrier. Preferably, a carrier is used, which is capable of increasing the efficacy of the agents of the invention to enter the target-cells. An aspect of the present invention further encompasses pharmaceutical compositions comprising one or more agents of the invention for administration to subjects in a biologically compatible form suitable for administration in vivo. The agents of the invention may be provided within delivery vehicles as described above that are formulated in a suitable pharmaceutical composition.

By “biologically compatible form suitable for administration in vivo” is meant a form of the substance to be administered in which any toxic effects are outweighed by the therapeutic effects. Administration of a therapeutically active amount of the pharmaceutical compositions of the present invention, or an “effective amount”, is defined as an amount effective at dosages and for periods of time, necessary to achieve the desired result of increasing/decreasing the production of proteins. A therapeutically effective amount of a substance may vary according to factors such as the disease state/health, age, sex, and weight of the recipient, and the inherent ability of the particular agent to elicit the desired response. Dosage regimen may be adjusted to provide the optimum therapeutic response. For example, several divided doses may be administered daily or at periodic intervals, and/or the dose may be proportionally reduced or increased as indicated by the exigencies of the therapeutic situation. The amount of agent for administration will depend on the route of administration, time of administration and varied in accordance with individual subject responses. Suitable administration routes are intramuscular injections, subcutaneous injections, intravenous injections or intraperitoneal injections, oral and intranasal administration .. The composition of the invention may also be provided via implants, which can be used for slow release of the composition over time.

The invention further provides a host cell comprising any of the vectors, such as recombinant expression vectors or viral vectors, described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α, E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell.

The pharmaceutically acceptable excipient is preferably one that is chemically inert to the miR, and/or mimics thereof and one that has little or no side effects or toxicity under the conditions of use. Such pharmaceutically acceptable carriers include, but are not limited to, water, saline, Cremophor EL (Sigma Chemical Co., St. Louis, MO), propylene glycol, polyethylene glycol, alcohol, and combinations thereof. The choice of carrier will be determined in part by the particular miR, and/or mimics thereof as well as by the particular method used to administer the composition. Accordingly, there is a wide variety of suitable formulations of the composition. When administered in the form of a liquid solution or suspension, the formulation can contain one or more of the active compounds and purified water. Optional components in the liquid solution or suspension include suitable preservatives (e.g., antimicrobial preservatives), buffering agents, solvents, and mixtures thereof. A component of the formulation may serve more than one function.

Preservatives may be used. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition. Suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition. The following formulations for oral, aerosol, parenteral (e.g., subcutaneous, intravenous, intraarterial, intramuscular, intradermal, interperitoneal, and intrathecal), and rectal administration are merely exemplary and are in no way limiting. Formulations of the present invention may be suitable for parental administration,

The agents of the present invention, alone or in combination with other suitable components, can be made into aerosol formulations to be administered via inhalation. The agents of the present invention may also be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3- dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations may include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets.

The agents of the invention may be administered as an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986). Topical formulations, including those that are useful for transdermal drug release, are well known to those of skill in the art and are suitable in the context of embodiments of the invention for application to the skin.

The concentration of a compound of embodiments of the invention in the pharmaceutical formulations can vary, e.g., from less than about 1%, usually at or at least about 10%, to as much as 20% to 50% or more by weight, and can be selected primarily by fluid volumes, and viscosities, in accordance with the particular mode of administration selected. Methods for preparing administrable (e.g., parenterally administrable) compositions are known or apparent to those skilled in the art and are described in more detail in, for example, Remington's Pharmaceutical Science (17th ed., Mack Publishing Company, Easton, PA, 1985).

When the agents of the invention are administered with one or more additional therapeutic agents, one or more additional therapeutic agents can be coadministered to the mammal. By “coadministering” is meant administering one or more additional therapeutic agents and the agents of the invention sufficiently close in time such that the agents of the invention can enhance the effect of one or more additional therapeutic agents. In this regard, the agents of the invention can be administered first and the one or more additional therapeutic agents can be administered second, or vice versa. Alternatively, the agents of the invention and the one or more additional therapeutic agents can be administered simultaneously. The additional therapeutic agent may be a recombinant expression vector comprising the wild type form of the coding sequence responsible for the inherited disease under the control of an appropriate promoter.

The delivery systems useful in the context of embodiments of the invention may include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain composition embodiments of the invention.

According to the present invention, for “prevention” is intended that administration of the agent decreases the chance of developing a disease or condition, i.e. it decreases the chance of developing a fibrosis and/or a disease associated with fibrosis. In some embodiments, for “prevention” is intended that administration of the agent stops or slows down progression of a disease that has already begun. For example, in some embodiments the agent of the invention is administered to a subject who already has fibrosis and the fibrosis does not develop to a more advanced stage. Stages of fibrosis can be classified according to standard methods known in the field such as the Ishak scale.

According to the present invention, for “treatment” it is intended that administration of the agent improves or cures or reverts a condition or a disease, i.e. it improves or cures or reverts a fibrosis or a disease associated with fibrosis. In some embodiments, for “treatment” it is intended that the disease, such as fibrosis, is not completely cured but it reverts to a less advanced stage.

In a preferred aspect of the method, the measurement of the amount of microRNA(s) is determined with a method comprising: RNA reverse transcription and/or nucleic acid hybridization and/or nucleic acid amplification and/or a combination thereof. The hybridization of the nucleic acids is preferably carried out using primers and/or probes, each of which is specific and selective for the sequence of one of the microRNAs defined above.

The amplification (and possible hybridization) of the nucleic acids is preferably carried out by quantitative real-time or digital PCR, more preferably comprising forward and reverse primers and optionally a probe.

In the method according to the invention the probe preferably comprises a sequence complementary to the sequences of the at least one miRNA as defined above.

The invention also relates to the use of a kit as defined above for carrying out a method as described above.

The amounts of miRNAs measured preferably correspond to levels of expression that are normalised. The measurement of the amount of miRNA is preferably performed by nucleic acid amplification and hybridization with primers and/or probes, each of which is specific and selective for the sequence of one of the microRNAs, preferably by qRT-PCR. Any other method for the detection and quantification of nucleic acids, such as digital PCR, microarrays or sequencing, is comprised within the scope of the invention.

The method according to the invention preferably comprises a step of extracting RNA from the biological sample. The RNA used to measure the levels of expression of the above-mentioned microRNAs is preferably extracted from a biological fluid sample or from a tissue sample, for example a biopsy or surgical piece.

In the kit according to the invention, detection means are understood to be sequence-specific amplification means and/or means for the quantitative detection of said amplified nucleic acids.

In the context of the present invention the detection means are preferably specific primers and/or probes for each miRNA to be detected. Optionally, the kit according to the invention comprises control means. A further aspect of the invention relates to a microarray or a PCR reaction plate for carrying out the method as described above, comprising specific probes for each miRNA to be detected.

A further object of the invention is a kit for carrying out the above-mentioned methods, comprising
means for detecting and/or measuring the amount of at least one microRNA as defined above and optionally,
control means.

Another object of the invention is a kit for detecting and/or measuring the amount of at least one microRNA as defined above, consisting of:
for each of said microRNAs, sequence-specific amplification means;
means for the quantitative detection of said amplified nucleic acids;
appropriate reagents.

A further object of the invention is a device for measuring the amount of at least one miRNA as defined above in a biological sample, wherein said device consists of:
solid support means, e.g. a microfluidic device, and
a system for detecting the amount of microRNA.

Said device is preferably a chip microarray, a microfluidic printed circuit board, QPCR tubes, QPCR tubes in a strip or a QPCR plate.

In the context of the present invention, the term “determining the level” or “detection” can also be understood as “measurement of the amount”. In the present invention, the expression “ measurement of the amount” can be understood as a measurement of the amount or concentration or level of the respective miRNA and/or the DNA thereof, preferably semi-quantitative or quantitative. The term “amount”, as used in the description, refers to, but is not limited to, the absolute or relative amount (or the level of concentration or expression) of miRNA and/or the DNA thereof, and any other value or parameter associated therewith or which can result therefrom. Methods for measuring miRNA and DNA in samples are well known in the art. For the purpose of detecting and/or measuring the levels of nucleic acid, the cells of the isolated biological sample can be lysed and the levels of miRNA in the lysates or purified or semi-purified RNAs from the lysates can be measured with any method known to the expert.

Such methods include hybridisation assays that use detectable marked DNA or RNA probes (for example Northern blotting) and/or nucleic acid amplification, for example quantitative or semi-quantitative RT-PCR methods, using appropriate oligonucleotide primers, e.g. LNA primers. The person skilled in the art knows how to design the appropriate primers. Alternatively, quantitative or semiquantitative in situ hybridization assays can be performed using, for example, tissue sections, or undried cell suspensions, and marked, detectable DNA or RNA probes (for example, fluorescent or marked with the enzyme). Further methods for the quantification of miRNA include digital PCR, small RNA sequencing and microRNA microarrays.

The methods of the invention can further comprise normalisation of the levels of expression of miRNA. Normalisation includes, but is not limited to, regulation of the levels of expression of miRNA with respect to the levels of expression of one or more nucleic acids in the isolated biological sample.

Although the tested miRNAs are indicated as RNA sequences, it will be understood that when reference is made to hybridisations or to other assays, corresponding DNA sequences can be used. For example, the RNA sequences can be reverse transcribed and amplified using a polymerase chain reaction (PCR) to facilitate detection. In these cases, DNA rather than RNA will actually be directly quantified. It will also be understood that the complementary strand of the reverse transcribed DNA sequences can be analysed rather than the sequence itself. In this context, the term “complementary” refers to an oligonucleotide that has an exactly complementary sequence, i.e. for every adenine there is a thymine, etc. Although the assays can be performed individually for the miRNAs, it is generally preferable to assay various miRNAs or compare the ratio of two or more miRNAs.

In the kit of the invention, “control means” are preferably used to compare the amount of microRNA with an appropriate control or an appropriate control amount. The “means for detecting and/or measuring the amount of microRNA” are known to the person skilled in the art and are preferably at least one marked, identifiable DNA or RNA probe specific for the miRNAs defined above and/or miRNA-specific primers for reverse transcribing or amplifying each of the aforesaid detected miRNAs. For example, said means can be specific TaqMan probes.

In the kit of the invention, the sequence-specific amplification means are known to the person skilled in the art and are preferably at least one DNA or RNA primer, e.g. a “stem-loop RT primer” or an LNA primer.

The design of probes or primers specific for miRNA is known to the person skilled in the art and appropriate probes and/or primers can be commercially available for purchase.

The kit of the invention can further comprise appropriate reagents, such as, for example, an enzyme for the preparation of cDNA (e.g. reverse transcriptase) and/or PCR amplification (e.g. Taq polymerase) and /or a reagent for detecting and/or quantifying miRNA. Moreover, the kit can further comprise a reagent for the isolation of miRNA from samples and/or one or more normalisation controls. The normalisation control can be provided, for example, as one or more separate reagents for marking the samples or the reactions. The normalisation control(s) is/are preferably selected from endogenous RNA or miRNA expressed in the sample.

The kit of the invention preferably comprises instructions for the interpretation of the data obtained.

In all the methods and embodiments presented herein, the sample isolated from the subject may be a body fluid e.g. it may be a sample of blood, of a blood-derived fluid (such as serum and plasma, in particular platelet-free plasma, e.g. a cell-free, citrate-derived platelet-free plasma sample), of saliva, of cerebrospinal fluid or of urine. In a particular embodiment, the body fluid is plasma or serum, deprived of platelets or not.

In the methods of the present invention, the body fluid level of the miR(s) in the subject may be compared to a reference level of the same miR. The “reference level” denotes a predetermined standard or a level determined experimentally in a sample processed similarly from a reference subject. Depending of the purpose of the methods of the present invention, the reference subject may be a healthy subject, a subject having a different disease from fibrosis or of diseases associated with fibrosis, or a subject with no liver fibrosis or diseases associated with fibrosis. The reference subject may also be a placebo treated patient. The reference level may also be the level of the same miR determined in a similarly processed body fluid sample obtained in the past from the same subject, allowing determining the evolution the fibrosis or of the disease associated with fibrosis in the subject, in particular allowing determining the evolution of the disease activity or fibrosis, or the efficiency of the treatment of the disease, depending on the method being implemented.

In a particular embodiment, the diagnosis and/or detection of fibrosis or of diseases associated with fibrosis, or the diagnosis and/or detection of a potential fibrosis or of diseases associated with fibrosis, in a subject is based on the detection of an increased level of miR34-b and/or miR34-c in the body fluid sample relative to a reference level measured in healthy subjects with no fibrosis or diseases associated with fibrosis.

In a particular embodiment, the diagnosis and/or detection of fibrosis or of diseases associated with fibrosis, in a subject is based on the detection of an increased level of miR34-b and/or miR34-c in the body fluid sample relative to a reference level measured in healthy subjects with no fibrosis or disease associated with fibrosis.

In another particular embodiment, the diagnosis and/or detection of a potential fibrosis or of diseases associated with fibrosis, in a subject is based on the detection of an increased level of miR34-b and/or miR34-c in the body fluid sample relative to a reference level measured in a non-fibrosis subject such as a healthy subject.

In another embodiment, the diagnosis and detection of significant fibrosis, or of potential significant liver fibrosis, in a subject is based on the detection of a decreased level of miR34-b and/or miR34-c in the body fluid sample relative to a reference level measured in a subject with minimal liver fibrosis.

In another embodiment, the diagnosis and detection of moderate fibrosis or of potential moderate fibrosis, in a subject is based on the detection of an increased level of miR34-b and/or miR34-c in the body fluid sample relative to a reference level measured in a subject with significant fibrosis.

The invention also provides a method for monitoring the evolution of fibrosis or of diseases associated with fibrosis stage in a subject, based on the evolution of the level of miR34-b and/or miR34-c in a body fluid sample of the subject relative to a reference level of the same miR from one or more body fluid sample(s) collected in the same subject in the past. In this method, an increase of the level of the miR indicates that the fibrosis increases whereas a decrease of the level of the miR indicates that the disease activity and fibrosis decline.

An increase of the level of miR34-b and/or miR34-c or a stable level of miR34-b and/or miR34-c indicates that the treatment is not efficient whereas a decrease of the level of miR34-b and/or miR34-c indicates that the treatment is efficient.

The invention further provides a method for predicting the response of a subject (e.g. prediction fibrosis stage) to a specific treatment (responder subject) based on the detection of a differential level of miR34-b and/or miR34-c in the body fluid sample relative to a reference level measured in a non-responder subject.

According to the invention, the amount of microRNA is preferably determined by detecting a nucleic acid comprising, respectively, SEQ ID NO: 1-4 or 9-12, a variant or isoform thereof or fragments thereof.

The nucleic acid variants can include nucleic acid sequences that have about 75%-99.9% of identity, in terms of nucleic acid sequence, with a nucleic acid sequence described here. Preferably, a variant nucleic acid sequence will have at least about 75%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.7%, 99.8% or 99.9% of nucleic acid sequence identity with respect to a nucleic acid sequence of an entire length or a fragment of a nucleic acid sequence as described here.

The term “fragments” comprises nucleic acid sequences that may be truncated at the 5′ end or 3′ end, or may lack internal residues but maintain their function. Fragments are preferably from 18 to 24 nt long.

In the present invention, reference is made indistinctly to microRNA, miRNA or hsa-miR or mmu-miR.

In the context of the present invention miR-34b include the following sequences (SEQ ID NOs. 1-4) and homologs and analogs thereof, miRNA precursor molecules, e.g. those disclosed below (SEQ ID NO:7-8), and DNA molecules encoding said miRNAs, e.g. those defined below (SEQ ID NO:5-6) and complementary nucleic acids.

In the context of the present invention miR-34c include the following sequences (SEQ ID NOs. 9-12) and homologs, orthologues, analogs and functional derivatives thereof, miRNA precursor molecules, e.g. those disclosed below (SEQ ID NO:15-16), and to DNA molecules encoding said miRNAs, e.g. those defined below (SEQ ID NO:13-14).

The identity of a homologue to a sequence of the sequences herein defined can preferably be at least 75%, or 80%, or 85%, or 90%, more preferably at least 95% identical, up to 99.9%.

SEQUENCES

>mmu-miR-34b-5p MIMAT0000382 (mature miRNA)
AGGCAGUGUAAUUAGCUGAUUGU (SEQ ID NO: 1)
>mmu-miR-34b-3p MIMAT0004581 (mature miRNA)
AAUCACUAACUCCACUGCCAUC (SEQ ID NO: 2)
>hsa-miR-34b-5p MIMAT0000685 (mature miRNA)
UAGGCAGUGUCAUUAGCUGAUUG (SEQ ID NO: 3)
>hsa-miR-34b-3p MIMAT0004676 (mature miRNA)
CAAUCACUAACUCCACUGCCAU (SEQ ID NO: 4)
>murine genomic sequence cloned in pAAV for miR-34b-5p
gcggccgcTCCGAGGGTTACTTGCACTTAgacctcgtgctcccggccctttgctgacgcatcctggctccggcctc
ggctttctgcggagtcagtggggctgcagcgctggcttctcctcccgcgggcggcgggtgatgctgtgccttgttttgatggcagtggag
ttagtgattgtcagcaccgcactacaatcagctaattacactgcctacaaaccgagcaccgggcgcccgccactgcagctcccgagggt
cgggcccctcgccccctttcgccacggtcgacaggcgagggcggcggagcgagaggtgcctcaggctcccgaggcccctccacac
ccagcagggccgcgcgcgaccccaggtgaacccccaggcgctgaggccccctgtccccgccgtcccccccgagacccccgactca
gcccggaccccagggcatccggccCGAGTCCTTCTTCCCGCAAggatcc (SEQ ID NO: 5)
MIR-34B (genomic sequence human)
GTGCTCGGTTTGTAGGCAGTGTCATTAGCTGATTGTACTGTGGTGGTTACAATCAC
TAACTCCACTGCCATCAAAACAAGGCAC (SEQ ID NO: 6)
>hsa-mir-34b MI0000742 (pre-miRNA)
GUGCUCGGUUUGUAGGCAGUGUCAUUAGCUGAUUGUACUGUGGUGGUUACAAU
CACUAACUCCACUGCCAUCAAAACAAGGCAC (SEQ ID NO: 7)
>mmu-mir-34b MI0000404 (pre-miRNA)
GUGCUCGGUUUGUAGGCAGUGUAAUUAGCUGAUUGUAGUGCGGUGCUGACAAU
CACUAACUCCACUGCCAUCAAAACAAGGCAC (SEQ ID NO: 8)
>mmu-miR-34c-5p MIMAT0000381 (mature miRNA)
AGGCAGUGUAGUUAGCUGAUUGC (SEQ ID NO: 9)
>mmu-miR-34c-3p MIMAT0004580 (mature miRNA)
AAUCACUAACCACACAGCCAGG (SEQ ID NO: 10)
>hsa-miR-34c-5p MIMAT0000686 (mature miRNA)
AGGCAGUGUAGUUAGCUGAUUGC (SEQ ID NO: 11)
>hsa-miR-34c-3p MIMAT0004677 (mature miRNA)
AAUCACUAACCACACGGCCAGG (SEQ ID NO: 12)
>murine genomic sequence cloned in pAAV for miR-34c-5p
gcggccgcAGTCAATATAATGACCAAATCAGCTAAGggataatttctatttttccaatatatctaaaaatcacaa
aaaatgtaccccacacaaattgatacattgtatacttagcagctaagggctagcggttccccccccccccccaaaccactaatagtatggt
aagaatatttccctatggctctgtcctcaccaaaatgacgattcacaggaggctcagtcggaggaatttcagtctttttacctggctgtgtgg
ttagtgattggtactattagcaatcagctaactacactgcctagtaactagactcagaaaaaagcatgcagtctttagctggtgctctcagac
tttggtgtgaccagagcaaatcgtcagccaagctgtggttgactctagtcgctgccttggtgatagctttctcagaagtggaaatcaggca
gtgaatcacagCAGCAGCAGGAACTGTTCTGGGatcc (SEQ ID NO: 13)
MIR34C (genomic sequence human)
AGTCTAGTTACTAGGCAGTGTAGTTAGCTGATTGCTAATAGTACCAATCACTAACC
ACACGGCCAGGTAAAAAGATT (SEQ ID NO: 14)
>hsa-mir-34c MI0000743 (pre-miRNA)
AGUCUAGUUACUAGGCAGUGUAGUUAGCUGAUUGCUAAUAGUACCAAUCACUA
ACCACACGGCCAGGUAAAAAGAUU (SEQ ID NO: 15)
>mmu-mir-34c MI0000403 (pre-miRNA)
AGUCUAGUUACUAGGCAGUGUAGUUAGCUGAUUGCUAAUAGUACCAAUCACUA
ACCACACAGCCAGGUAAAAAGACU (SEQ ID NO: 16)
hsa-miR-34b/c primary transcript (pri-miRNA)
AGCGGAGGCCAAAUCAACAGCAACCCUAAGAACAAGCAUUCUUUUUUUUUUUU
CAACAGAACUAGGCCACACAUAUUUUU
UGUCGUUAUUUAAAUUUUUAGUUGUACUACAUAGAAAAUAAACUCAUUUUAAA
AAGUUAUUUCAGUAGGCAAUGCAUCUU
CAUGACUUUUACAUAUGAGUUUUAUUUUUUAUUACUUUUGAAGAAAAGUCUGU
AGAAACCUACUUUUCAAGGCAUCUGAC
CCAACCAUUGCCUUGGAUGGCAGCAAUCCAGCUCAGGCACAGCAUCACCGCCGC
CCGGCCGGGAAGAAGACGCCGGCUCG
GGUAGCCCGCAGCCUUCGAGAGAAGAUGCCUGAGAAGCGCGGCGUCGGCGUGG
GUCCUGCGCAGCCUGCCCCGCGAGCGC
CCGCUGCAAGUGCGAGGAAACCCGCGGUUUCUCCAGAUACAGUUAAACUGUUA
GCUCUCUCUAGGAGUCACAGAAGAUGA
AACAGUCUCAUGCCAGGAAAGCAAAAUCCCUGGAGGUGAAGCCCCUCCAUCCA
UGUAACAGUUAAUACUGUAUGCUGUGA
UUCACUGUGUCUAUUUGCCAUCGUCUAGUAGAGUAUUCACCAAGCUAGCAACU
CAGUUGAGCUCCAACUCAACCAAUGAA
UUGCCUGCCUGUCACAACGUGUUGGGGUACCAACUUGAGACUGCAAUUUUUUC
UAUGAGUCUAGUUACUAGGCAGUGUAG
UUAGCUGAUUGCUAAUAGUACCAAUCACUAACCACACGGCCAGGUAAAAAGAU
UUGGGAAUUCGUCCAAAUGAGCUGCCU
GUGCAUCAUCAAUGUGCGUGGGGAAGAGGGGUGUUGGAAAAUGCUGAUUUCAU
CCAUUGCCUAUUAAUUGCUCAGCCAAA
AGAAAAAAAUCAACAUUUCAGCUACUAAGUUUACAAUGUAUGUAAUGUGUAUG
UAUGUGGGGUUUUGUUUUGUUUUGUUU
UCAAUAUUCCUUCAGGCUCUUAACCAAAAUUUUAGAUAUAAGGGGGAAUAUGA
UUUUUUUCUUAGCUGACUGAUGUAUGU
UAUUAUAUGAACAUGUGAUUAUUAACUUCUUGAGACUAUAUUGUUAGUAAUA
UUUUGAAAGUAAUAUUGUUAGUAAUAUU
UCGAAAGAAUAAAGUGCCAUAAAGACAAAAAAAAAAAAAAAA (SEQ ID NO: 17)
hsa-miR-34b/c primary transcript (pri-miRNA) (DNA sequence)
AGCGGAGGCCAAATCAACAGCAACCCTAAGAACAAGCATTCTTTTTTTTTTTTCAACAGAACTAGGCCACACATA
TTTTTTGTCGTTATTTAAATTTTTAGTTGTACTACATAGAAAATAAACTCATTTTAAAAAGTTATTTCAGTAGGCA
ATGCATCTTCATGACTTTTACATATGAGTTTTATTTTTTATTACTTTTGAAGAAAAGTCTGTAGAAACCTACTTTTC
AAGGCATCTGACCCAACCATTGCCTTGGATGGCAGCAATCCAGCTCAGGCACAGCATCACCGCCGCCCGGCCG
GGAAGAAGACGCCGGCTCGGGTAGCCCGCAGCCTTCGAGAGAAGATGCCTGAGAAGCGCGGCGTCGGCGTG
GGTCCTGCGCAGCCTGCCCCGCGAGCGCCCGCTGCAAGTGCGAGGAAACCCGCGGTTTCTCCAGATACAGTTA
AACTGTTAGCTCTCTCTAGGAGTCACAGAAGATGAAACAGTCTCATGCCAGGAAAGCAAAATCCCTGGAGGTG
AAGCCCCTCCATCCATGTAACAGTTAATACTGTATGCTGTGATTCACTGTGTCTATTTGCCATCGTCTAGTAGAG
TATTCACCAAGCTAGCAACTCAGTTGAGCTCCAACTCAACCAATGAATTGCCTGCCTGTCACAACGTGTTGGGGT
ACCAACTTGAGACTGCAATTTTTTCTATGAGTCTAGTTACTAGGCAGTGTAGTTAGCTGATTGCTAATAGTACCA
ATCACTAACCACACGGCCAGGTAAAAAGATTTGGGAATTCGTCCAAATGAGCTGCCTGTGCATCATCAATGTGC
GTGGGGAAGAGGGGTGTTGGAAAATGCTGATTTCATCCATTGCCTATTAATTGCTCAGCCAAAAGAAAAAAAT
CAACATTTCAGCTACTAAGTTTACAATGTATGTAATGTGTATGTATGTGGGGTTTTGTTTTGTTTTGTTTTCAATA
TTCCTTCAGGCTCTTAACCAAAATTTTAGATATAAGGGGGAATATGATTTTTTTCTTAGCTGACTGATGTATGTTA
TTATATGAACATGTGATTATTAACTTCTTGAGACTATATTGTTAGTAATATTTTGAAAGTAATATTGTTAGTAATA
TTTCGAAAGAATAAAGTGCCATAAAGACAAAAAAAAAAAAAAAA (SEQ ID NO: 18)
Mir34a (murine genomic sequence)
CCAGCTGTGAGTAATTCTTTGGCAGTGTCTTAGCTGGTTGTTGTGAGTATTAGCTA
AGGAAGCAATCAGCAAGTATACTGCCCTAGAAGTGCTGCACATTGT
(SEQ ID NO: 19)
mmu-miR-34a (murine pre-miRNA)
CCAGCUGUGAGUAAUUCUUUGGCAGUGUCUUAGCUGGUUGUUGUGAGUAUUAG
CUAAGGAAGCAAUCAGCAAGUAUACUGCCCUAGAAGUGCUGCACAUUGU (SEQ
ID NO: 20)
mmu-miR-34a-5p (murine mature miRNA)
UGGCAGUGUCUUAGCUGGUUGU (SEQ ID NO: 21)
MIR34A (genomic sequence human)
GGCCAGCTGTGAGTGTTTCTTTGGCAGTGTCTTAGCTGGTTGTTGTGAGCAATAGT
AAGGAAGCAATCAGCAAGTATACTGCCCTAGAAGTGCTGCACGTTGTGGGGCCC
(SEQ ID NO: 22)
hsa-miR-34a (pre-miRNA)
GGCCAGCUGUGAGUGUUUCUUUGGCAGUGUCUUAGCUGGUUGUUGUGAGCAAU
AGUAAGGAAGCAAUCAGCAAGUAUACUGCCCUAGAAGUGCUGCACGUUGUGGG
GCCC (SEQ ID NO: 23)
hsa-miR-34a-5p (mature miRNA)
UGGCAGUGUCUUAGCUGGUUGU (SEQ ID NO: 24)

The present invention will now be illustrated with non-limiting examples in reference to the following figures.

### BRIEF DESCRIPTION OF THE FIGURES

Embodiments and experiments illustrating the principles of the invention will be discussed with reference to the following figures:

FIG. 1. miR-34b/c^{-/-} mice are more prone to develop thioacetamide-induced liver fibrosis. A) Representative hematoxylin and eosin and Sirius red staining of livers from C57BL/6 wild-type (WT) and miR-34b/c^{-/-} mice treated with thioacetamide (TAA) (n=8 per group) or vehicle (n=5 per group). Scale bar: 100 μm B) Quantitative morphometry of Sirius Red (SR) staining. Data are expressed as percentage over total field area. C) Liver hydroxyproline (HYP) content. D) qPCR analysis of fibrosis marker genes Acta2, Tgfb1 and Timp1. E) qPCR analysis of inflammation genes Il6 and Ccl2. F) Levels of serum alanine aminotransferase (ALT) in wild-type (WT) or miR-34b/c^{-/-} mice treated with thioacetamide (TAA). Two-way ANOVA plus Tukey's post-hoc: * p<0.05; ** p<0.01; *** p<0.005.

FIG. 2. miR-34b/c^{-/-} mice are more prone to develop carbon tetrachloride-induced liver fibrosis. A) Representative hematoxylin and eosin and Sirius red staining of liver from C57BL/6 wild-type (WT) and miR-34b/c^{-/-} mice treated with carbon tetrachloride (CCl₄) (n=11 per group) or vehicle (n=10-13 per group). Scale bar: 100 μm B) Quantitative morphometry of Sirius Red (SR) staining. Data are expressed percentage over total field area. C) Liver hydroxyproline (HYP) content. D) qPCR analysis of fibrosis marker genes Col1a1, Tgfb1 and Timp1. E) qPCR analysis of inflammation genes (cl2 and Il6. F) Levels of serum alanine aminotransferase (ALT) in wild-type (WT) or miR-34b/c^{-/-} mice treated with carbon tetrachloride (CCl₄). Two-way ANOVA plus Tukey's post-hoc: * p<0.05; ** p<0.01; *** p<0.005.

FIG. 3. miR-34b/c mimic antagonizes human stellate cells activation. A) Representative western blot on total lysates from Huh-7 and LX-2 co-cultures transfected with transfection reagent alone (TR), miRNA mimic negative control (NC), or human miR-34b/c mimic (miR) and treated with human Transforming Growth Factor β1 (TGF-β1, 2 ng/μL) or with vehicle (n=6 per group). B-E) Quantification of band intensities from western blot (n=6 per group). Two-way ANOVA plus Tukey's post-hoc: * p<0.05; ** p<0.01; *** p<0.005.

FIG. 4. Hepatic delivery of miR-34b/c ameliorates thioacetamide-induced advanced liver fibrosis. A) Schematic representation of treatment schedule. B) Representative hematoxylin and eosin and Sirius red staining of livers from C57BL/6 wild-type mice treated with thioacetamide (TAA) or vehicle (n=5) and injected with adeno-associated vector expressing either miR-34b/c (AAV-miR-34b/c) (n=10) or GFP as control (AAV-GFP) (n=9). Scale bar: 100 μm. C) Quantitative morphometry of Sirius Red (SR) staining. Data are expressed percentage over total field area. D) Liver fibrosis staging according to Ishak scoring system. E) Liver hydroxyproline (HYP) content. F) Expression of fibrosis marker genes Acta2, Col1a1 and Timp1 by qPCR. One-way ANOVA plus Tukey's post-hoc or Kurskal-Wallis plus Dunn's multiple comparison (C only): * p<0.05; ** p<0.01; *** p<0.005.

FIG. 5. Inflammation and hepatocellular damage in thioacetamide-induced advanced liver fibrosis. A) Necroinflammation activity according to Ishak scoring system in livers from C57BL/6 wild-type mice treated with thioacetamide (TAA) or vehicle and injected with adeno associated vector expressing miR-34b/c (AAV-miR-34b/c) or GFP as control (AAV-GFP). Kurskal-Wallis plus Dunn's multiple comparison: * p<0.05. B) qPCR analysis of inflammation genes Ccl2 and Il6. C) Serum alanine amino transferase (ALT) levels. One-way ANOVA plus Tukey's post-hoc: ** p<0.01.

FIG. 6. miR-34b/c overexpression ameliorates carbon tetrachloride-induced advanced liver fibrosis. A) Schematic representation of treatment schedule. B) Representative hematoxylin and eosin and Sirius red staining of livers from C57BL/6 wild-type mice treated with carbon tetrachloride (CCl₄) or vehicle (n=5) and injected with adeno associated vector expressing miR-34b/c (AAV-miR-34b/c) (n=7) or GFP as control (AAV-GFP) (n=5). Scale bar: 100 μm. C) Quantitative morphometry of collagen area by Sirius Red (SR) staining. Data are expressed percentage over total field area. D) Liver fibrosis staging according to Ishak scoring system E) Liver hydroxyproline (HYP) content. F) Expression of fibrosis genes Acta2, Col1a1 and Tgfb1 by qPCR. One-way ANOVA plus Tukey's post-hoc Kurskal-Wallis plus Dunn's multiple comparison (C only): * p<0.05; ** p<0.01; *** p<0.005.

FIG. 7. Inflammation and hepatocellular damage in carbon tetrachloride-induced advanced liver fibrosis. A) Necroinflammation activity according to Ishak scoring system in livers from C57BL/6 wild-type mice treated with carbon tetrachloride (CCl₄) or vehicle and injected with adeno associated vector expressing miR-34b/c (AAV-miR-34b/c) or GFP as control (AAV-GFP). Kurskal-Wallis plus Dunn's multiple comparison: * p<0.05; ** p<0.01. B) qPCR analysis of inflammation genes Ccl2 and Tnfa. Infa is shown instead of Il6 whose expression was below the limit of detection for several samples. C) Serum alanine aminotransferasi (ALT) levels. One-way ANOVA plus Tukey's post-hoc.

FIG. 8. Hierarchical clustering and heatmap of differentially expressed miRNAs from next generation sequencing analysis of PiZ versus wild-type (WT) livers (n=5 per group). The miR-34b-5p and miR-34c-5p are framed in red.

FIG. 9. Increased expression of miR-34b/c in mouse livers expressing Z α1-antitrypsin. (A) Differentially expressed miRNAs visualized by volcano plot. miRNAs with FDR<10⁻² (y-axis) and fold change in PiZ over wild-type (WT) >|4| are shown and miR-34bc/c are red circled. (B) qPCR on PiZ versus WT for miR-34 family members on liver RNA showing significant upregulation of miR-34b/c (at least n=3 per group; t-test: * p<0.05, ** p<0.01 versus WT). (C) qPCR on PiZ versus WT for miR-34 family members on plasma RNA showing significant upregulation of miR-34a-5p, miR-34b-3p and 5p, and miR-34c-5p (n=4 per group; t-test: ** p<0.01, *** p<0.005 versus WT).

FIG. 10 Circulating miR-16 levels as a marker of hemolysis. Difference in miR-16 levels between PiZ and wild-type plasma is not significant (n=4-5 per group; t-test).

FIG. 11. Expression levels of miR-34b/c correlate with Z α1-antitrypsin accumulation. (A) Expression of pri-miR-34b/c and albumin (Alb) in parenchymal and non-parenchymal cells from PiZ mouse livers (n=3; 1-test: * p<0.05, *** p<0.005) (B) Representative image of microdissected PAS-D⁺ and PAS-D⁻ areas in PAS-D stained livers from PiZ mice. (C) qPCR for miR-34b/c on micro-dissected liver areas with ATZ globule accumulation (PAS-D+, red dotted lines in B) or devoid of ATZ globules (PAS-D-, blue dotted lines in B) showed enrichment of miR-34b/c in ATZ rich areas (n=4 per group; paired /-test: * p<0.05 and ** p<0.01 versus PAS-D″). (D) qPCR for miR-34b/c in PiZ livers injected with rAAV-miR914 or control vector showing reduction of miR-34b/c levels in rAAV-miR914 injected mice at four weeks post-injection (n=5 per group; t-test: * p<0.05 versus rAAV-GFP).

FIG. 12. FOXO3 activation in PiZ livers. (A) Western blot and (B) quantification of band intensities for FOXO3 in PiZ and wild-type livers. β-actin (ACTB) was used as loading control. (C) Representative FOXO3 immuno-histochemistry on livers from wild-type (WT) and PiZ mice showing increased nuclear localization of FOXO3. Yellow arrow heads point to FOXO3 positive nuclei. □central vein; _{—} portal vein (n=3 per group; magnification: left panels, 20X; middle and right panels, 40X; scale bar: 100 μm). (D) Western blot and (E) quantification of band intensities (t-test: *** p<0.005) on liver nuclear extracts showing increased FOXO3 in PiZ compared to WT livers. RAD50 is used for nuclear protein normalization; GAPDH is shown as control for purity of nuclear extracts. CYT: cytoplasm fraction. (F) Enrichment plot and (G) summary of results from Gen Set Enrichment Analysis (GSEA) including FOXO3 target genes showing enrichment in PiZ mice versus wild-type livers.

FIG. 13. JNK-mediated FOX03 activation and upregulation of miR-34b/c in PiZ livers. (A) Western blot and (B) quantification of band intensities (one-way ANOVA and Tukey's post-hoc test: * p<0.05) on whole liver extracts showing increased phospho-Ser⁵⁷⁴ FOXO3 in PiZ compared to WT mice and reduced levels of phosphorylated FOXO3 in PiZ/Jnkl^{-/-} mice compared to PiZ and WT controls. (C) qPCR for FOXO3 in wild-type, PiZ and PiZ/Jnkl^{-/-} livers showing no significant differences (n=4 per group at least; one-way ANOVA). (D) Western blot and (E) quantification of band intensities (one-way ANOVA and Tukey's post-hoc test: *p<0.05) on liver nuclear extracts showing decreased FOXO3 in PiZ/Jnkl^{-/-} nuclei compared to PiZ livers. H3 is used for nuclear protein normalization; GAPDH is shown as control for purity of nuclear extracts. CYT: cytoplasm fraction. (F) qPCR for miR-3b/c showing significant downregulation of miR-34b/c levels in livers of PiZ/Jnkl^{-/-} compared to PiZ mice (n=5 per group; one-way ANOVA and Tukey's post-hoc test: ** p<0.01 versus PiZ).

FIG. 14. Deletion of miR-34b/c resulted in early development of liver fibrosis in PiZ mice. (A) Representative PAS-D and Sirius Red stainings of livers from wild-type (WT), miR-34b/c^{+/-}, PiZ/miR-34b/c^{+/+} as controls, PiZ/ miR-34b/c^{+/-}, and PiZ/miR-34b/c^{-/-} showing similar ATZ accumulation by PAS-D in PiZ/miR-34b/c^{+/+}, PiZ/ miR-34b/c^{+/-}, and PiZ/miR-34b/c^{-/-} and increased fibrosis in PiZ/miR-34b/c^{+/-} and PiZ/miR-34b/c^{-/-} compared to controls. (B) Serum levels of alanine aminotransferase (ALT) in wild-type, miR-34b/c^{-/-}, PiZ/miR-34b/c^{+/+}, and PiZ/miR-34b/c^{-/-} (n=5 to 13 per group; one-way ANOVA and Tukey's post-hoc test) (C) Quantification of percentages of Sirius Red (SR) positive area showing increased stained area in PiZ/miR-34b/c^{-/-} compared to controls (n=5 image per animal, n=3 to 11 animals per group; one-way ANOVA and Tukey's post-hoc test: ** p<0.01). (D) Hydroxyproline (HYP) determination on liver lysates showing increased hydroxyproline content in PiZ/miR-34b/c^{-/-} compared to controls (n=5 to 11 per group; one-way ANOVA and Tukey's post-hoc test: *p<0.05).

FIG. 15. Deletion of miR-34b/c dysregulates liver fibrosis associated genes in PiZ mice. (A) Principal component analysis of transcriptomic data from wild-type, miR-34b/c^{-/-}, PiZ/miR-34b/c^{+/+}, and PiZ/miR-34b/c^{-/-} mouse livers. (B) Top five upregulated cellular processes (upper panel) and biological components (lower panel) from Gene Ontology analysis on differentially expressed genes in PiZ/miR-34b/c^{-/-} versus PiZ/miR-34b/c^{+/+} livers. (C) Expression of liver fibrosis gene signature in wild-type (WT), miR-34b/c^{-/-}, PiZ/miR-34b/c^{+/+},and PiZ/miR-34b/c^{-/-}. Each column represents the average gene expression levels of n=5 mice per group. (D) Summary of results and enrichment plot from Gene Set Enrichment Analysis (GSEA) including liver fibrosis gene signature showing enrichment in PiZ mice versus wild-type livers.

FIG. 16. VENN diagram comparing gene datasets of miR-34b/c^{-/-} versus wild-type and PiZ/miR-34b/c^{-/-} versus PiZ/miR-34b/c^{+/+} at 13 to 15 weeks of age. Genes considered for further studies are highlighted in yellow. Abbreviations: DEG, differentially expressed genes; DW, downregulated; UP, upregulated; WT, wild-type.

FIG. 17. Increased PDGF signaling in PiZ/miR-34b/c^{-/-} livers. Schematic representation of miR-34/c binding to 8-mer recognition sites in Pdgfra (A) and Pdgfrb (B) 3′UTRs. miR-34b/c seed sequence pairings are depicted in blue and other base pairings are in red. Nucleotides that have been mutated for luciferase assays are indicated by asterisks. Luciferase activity assay on HeLa cells transfected with negative control (NC), miR-34b, or miR-34c mimic and with luciferase expressing plasmids carrying wild-type (WT) or mutated (mut) Pdgfra (C) and Pdgfrb (D) 3′UTR. (n=6 per group; one-way ANOVA and Tukey's post-hoc test: ** p<0.01, *** p<0.005). (E) Western blotting of PDGF pathway on whole liver extracts showing increased levels of miR-34b/c target genes PDGFRα/β, activation of PDGFRα/β and phosphorylation of PDGFR target proteins JAK1 and AKT in PiZ/miR-34b/c^{-/-} versus PiZ/miR-34b/c^{+/+} mice. (F) Quantification of band intensities of western blots in E (t-test: ** p<0.01, *** p<0.005).

FIG. 18. FOXO3 activation and miR-34c upregulation in livers of patients with AAT deficiency. (A) Western blot and (B) quantification of band intensities (t-test: * p<0.05) of liver nuclear extracts from AAT deficiency patients who underwent liver transplantation (Pi*ZZ) compared to control liver samples from patients undergoing liver transplantation for unrelated liver causes (Pi*MM) showing increased nuclear FOXO3 in livers from Pi*ZZ subjects. (C) Expression of miR-34c by qPCR is significantly increased in liver samples from Pi*ZZ subjects (Pi*ZZ, n=5) compared to control liver samples (Pi*MM, n=4). (1-test: ** p<0.01). (D) Western blot and (E) quantification of band intensities on whole liver extracts from AAT deficiency patients with mild liver disease (AATD) and from control subjects with unrelated liver disorders, showing increase phospho-Ser574 FOXO3 in livers of AAT deficiency patients with mild liver disease (t-test: * p<0.05). (F) Hepatic miR-3-4c levels by qPCR in AAT deficiency patients with mild liver disease (AATD) (n=4) and control subjects (n=2) showing a trend in miR-34c upregulation for AATD sample (t-test). (G) Representative image of laser microdissected PAS-D⁺ (red dotted lines) and PAS-D″ (blue dotted lines) areas in PAS-D stained liver from a Pi*ZZ subject. (H) qPCR for miR-34c on laser micro-dissected liver areas from a single AAT deficiency patient showing enrichment in ATZ accumulating livers areas (PAS-D+), versus liver areas devoid of ATZ globules (PAS-D-) (t-test: ** p<0.01).

FIG. 19. JNK-mediated FOXO3 activation in liver fibrosis. Western blot analysis and quantification of band intensities for total and phosphorylated JNK and FOX03 on whole liver extracts from (A, B) Abcb4^{-/-} mice, (C, D) mice with bile duct ligation (BDL), (E, F) mice treated with thioacetamide (TAA) or (G,H) carbon-tetrachloride (CCl₄) versus controls (n=4 for each group, t-test: * p<0.05; *** p<0.005).

FIG. 20. miR-34b/c upregulation in liver fibrosis. qPCR for miR-34b/c on whole liver extracts from (A) Abcb4^{-/-} mice, (B) mice with bile duct ligation (BDL), (C) mice treated with thioacetamide (TAA) or (D) carbon-tetrachloride (CCl₄) versus controls (n=4 for each group, t-test: * p<0.05; *** p<0.005).

FIG. 21.Thioacetamide-induced liver fibrosis in miR-34b/c^{-/-} mice. A) Western blot analysis on liver extracts from wild-type and miR-34b/c^{-/-} mice treated with thioacetamide (TAA) (n=3 per group). Calnexin (CNX) was used as loading control. B) Quantification of band intensities from western blots in panel A.

FIG. 22. Carbon tetrachloride-induced liver fibrosis in miR-34b/c^{-/-} mice A) Western blot analysis on liver extracts from wild-type and miR-34b/c″- mice treated with carbon tetrachloride (CCl₄) (n=3 per group). Calnexin (CNX) was used as loading control.B) Quantification of band intensities from western blots in panel A

FIG. 23. Transcriptional analysis of miR-34b/c-treated LX-2 cells. (A) Principal component analysis of transcriptomic data from LX2 cells treated with vehicle or Transforming Growth Factor B1 (TGF-β1) and left un-transfected or transfected with miRNA mimic negative control (NC) or with human miR-34b/c mimics (miR-34b/c) (n=4-5 per group). (B) VENN diagram comparing differentially expressed genes in LX-2 cells treated with TGF-B1+NC versus vehicle+NT and TGF-β1+miR-34b/c versus TGF-β1+NC. Abbreviations: DEG, differentially expressed genes; DW, downregulated; UP, upregulated;

FIG. 24. miR-34b/c inhibits human stellate cells activation. (A) Biological processes (upper panel) and cellular components (lower panel) from clustered gene ontology analysis on differentially expressed genes in opposite correlation in human Transforming Growth Factor β1 TGF-β1)+negative control versus vehicle-treated or un-transfected LX2 cells and TGF-β1+miR-34b/c versus TGF-β1+negative control treated LX2 cells. (B) Gene Set Enrichment Analysis using activated (left panel) and quiescent hepatic stellate cells (HSC) gene sets on transcriptomic data from LX2 cells treated with TGF-β1+miR-34b/c versus TGF-β1+negative control-treated cells.

FIG. 25. miR-34b/c targets COLIAl and genes of collagen biosynthesis. (A) Schematic representation of human miR-34b and -34c binding to 7-mer recognition sites in pro-α-1 chain collagen type I gene 3′UTRs. Nucleotides that have been mutated for luciferase assays are indicated by asterisks. (B) Luciferase activity assay on HeLa cells transfected with negative control (NC), miR-34b, or miR-34c mimic and with plasmids expressing luciferase gene carrying wild-type (WT) or mutated (mut) COL1A1 3′-UTR (n=3 per group; one-way ANOVA and Tukey's post-hoc test: ** p<0.01, *** p<0.005). (C) Enrichment plot from Gene Set Enrichment Analysis using collagen biosynthesis gene signature on transcriptomic data from LX2 cells treated with human Transforming Growth Factor β1 (TGF-β1)+miR-34b/c versus TGF-β1+negative control—treated cells. (D) Expression heatmap of collagen biosynthesis gene signature from LX2 cells incubated with vehicle (n=4) or human Transforming Growth Factor β1 (TGF-β1) and transfected with miRNA mimic negative control (NC) (n=4), or human miR-34b/c mimics (miR) (n=5).

FIG. 26. Hepatic delivery of miR-34b/c ameliorates thioacetamide-induced liver fibrosis. (A) Representative hematoxylin and eosin and Sirius red staining of livers from C57BL/6 wild-type mice treated with thioacetamide (TAA) and injected with adeno-associated vector expressing miR-34b (AAV-miR-34b) (n=7) or miR-34c (AAV-miR-34c) (n=9). Scale bar: 100 μm. (B) Quantitative morphometry of Sirius Red (SR) staining in livers from C57BL/6 wild-type mice treated with TAA or vehicle and injected with AAV-miR-34b, AAV-miR-34c, or both AAV-miR-34b/c or AAV-GFP as control. Data are expressed as percentage over total field area.

(C) Liver fibrosis staging according to Ishak's scoring system. (D) Liver hydroxyproline (HYP) tent. (E) Necro-inflammation grading according to Ishak's scoring system. (F) Serum alanine aminotransferase (ALT) (n=5-7 per group). One-way ANOVA plus Tukey's post-hoc or Kurskal-Wallis plus Dunn's multiple comparison (C only): * p<0.05; ** p<0.01; *** p<0.005; *** *p<0.001; ns, not significant.

FIG. 27. Hepatic delivery of miR-34b/c ameliorates carbon tetrachloride-induced liver fibrosis. (A) Representative hematoxylin and eosin and Sirius red staining of livers from C57BL/6 wild-type mice treated with thioacetamide (CCl₄) and injected with adeno-associated vector expressing miR-34b (AAV-miR-34b) (n=7) or miR-34c (AAV-miR-34c) (n=5). Scale bar: 100 μm. (B) Quantitative morphometry of Sirius Red (SR) staining in livers from C57BL/6 wild-type mice treated with CCl₄ or vehicle and injected with AAV-miR-34b, AAV-miR-34c, or both AAV-miR-34b/c or AAV-GFP as control. Data are expressed as percentage over total field area. (C) Liver fibrosis staging according to Ishak's scoring system. (D) Liver hydroxyproline (HYP) content. (E) Necro-inflammation grading according to Ishak's scoring system. (F) Serum alanine aminotransferase (ALT) (n=5-7 per group). One-way ANOVA plus Tukey's post-hoc or Kurskal-Wallis plus Dunn's multiple comparison (C only): * p<0.05; ** p<0.01; *** p<0.005; **** p<0.001

FIG. 28. Liver-directed delivery of miR-34b/c reduced expression of COL1A1 and PDGFR-α/β. (A) Col1a1 expression by qPCR analysis in livers from C57BL/6 wild-type mice treated with thioacetamide (TAA) or vehicle (n=5) (upper panel) or CC1-4 or vehicle (lower panel) and injected with adeno-associated vector expressing either miR-34b/c (AAV-miR-34b/c) (n-7-10) or GFP as control (AAV-GFP) (n=5-9). (B) Representative immunohistochemistry for COL1A1 (n=5 per treatment). Scale bar: 100 μm. C) Western blot analysis for PDGFR-α and PDGFR-β of liver lysates from TAA-(upper panel) and CCl₄-treated animals (lower panel). Calnexin (CNX) was used as loading control. D) Quantification of band intensities from western blots in panel A

### TABLES

TABLE 1
Primers used for qPCR analysis

| Gene | Forward (5′-3′) | Reverse (5′-3′) | SEQ ID NO: |
|---|---|---|---|
| Il6 | CCGGAGAGGAGACTTCACAG | CAGAATTGCCATTGCACAAC | 25 |
| Tgfb1 | TTGCTTCAGCTCCACAGAGA | CAGAAGTTGGCATGGTAGCC | 26 |
| Ccl2 | GCTCAGCCAGATGCAGTTAA | TCTTGAGCTTGGTGACAAAAACT | 27 |
| Col1a1 | GCCAAGAAGACATCCCTGAA | GCCATTGTGGCAGATACAGA | 28 |
| Timp1 | CTCATCACGGGCCGCCTAAG | CACTGTGCACACCCCACAGC | 29 |
| Acta2 | CCTGGCTTCGCTGTCTACCT | TTGCGGTGGACGATGGA | 30 |
| Tnfa | CTGAACTTCGGGGTGATCGG | GGCTTGTCACGAATTTTGAGA | 31 |
| B2m | TGGTGCTTGTCTCACTGACC | GTATGTTCGGCTTCCCATTC | 32 |

TABLE 2
Antibodies used for western blot and IHC analyses

| Antibody | Manufacturer | Dilution |
|---|---|---|
| anti-PDGFRα | Cell Signaling | 1/500 |
| | Technologies (#3174) | |
| anti-PDGFRβ | Cell Signaling | 1/500 |
| | Technologies (#3169) | |
| anti-phospho-PDGFRα/β | Cell Signaling | 1/500 |
| (Tyr849/Tyr857) | Technologies (#3170) | |
| anti-Actin, α-Smooth Muscle | Merk | 1/1000 |
| (Thr183/Tyr185) | (A5228) | |
| anti-Collagen I | Abcam | 1/250 (WB) |
| | (ab138492) | 1/100 (IHC) |
| anti-Calnexin | Santa Cruz Biotecnology | 1/1000 |
| | (sc-46669) | |

TABLE 3
Clinical features of AAT deficiency and
control patients with mild liver disease.

| Patient | Age | Gender | Other known conditions |
|---|---|---|---|
| AATD#1 | 83 | M | chronic hepatitis, alcoholic |
| | | | liver damage |
| AATD#2 | 90 | M | chronic hepatitis |
| AATD#3 | 66 | M | chronic hepatitis |
| AATD#4 | 70 | M | chronic sclerosis, steatohepatitis, |
| | | | eosinophil infiltration |
| control#1 | 52 | M | rectal carcinoma metastasis |
| control#2 | 67 | M | colon adenocarcinoma |

TABLE 4
TaqMan microRNA assays.

| miRNA | Assay ID |
|---|---|
| mmu-miR-34a-5p | 000426 |
| mmu-miR-34a-3p | 465771_mat |
| mmu-miR-34b-5p | 002617 |
| mmu-miR-34b-3p | 002618 |
| mmu-miR-34c-5p | 000428 |
| mmu-miR-34c-3p | 002584 |
| pri-miR-34b/c | Mm03306660_pri |
| mmu-miR-216b-5p | 002326 |
| mmu-miR-216a-3p | 464267_mat |
| mmu-miR-217-5p | 002556 |
| mmu-miR-708-5p | 002341 |
| mmu-miR-154-3p | 000478 |
| mmu-let-7b-3p | 002404 |
| mmu-miR-1948-3p | 121171_mat |
| mmu-miR-664-3p | 001323 |
| mmu-miR-23a | 000399 |
| mmu-miR-16 | 000391 |
| snoRNA234 | 001234 |
| U47 | 001223 |
| 18s | Hs03003631_g1 |
| hsa-miR-152 | 000475 |

TABLE 5
Primers used for Pdgra and Pdfrb 3′UTR cloning and mutagenesis of
miR-34b/c target sites.

| | | | SEQ ID |
|---|---|---|---|
| Primer | Sense | Sequence (5′ > 3′) | NO: |
| Pdgfra | F | GCTCGCTAGCCTCGAGCTGACACGCTCCGGGTATCAT | 33 |
| 3′UTR | R | ATGCCTGCAGGTCGACAAGTCATATATAATAAATCATTTAT | 34 |
| Pdgfrb | F | TAGCCTCGAGTCTAGAGAACTGACATCACTCCATTTTGCCC | 35 |
| 3′UTR | R | ATGCCTGCAGGTCGACCGGTTATTCAGTGAGAAGCACC | 36 |
| Pdgfra | F | gttaaaaaaaatataaacaaaagccgtaatacagcttgtcata | 37 |
| site | | cacattttggcagtattctccaa | |
| mut | R | ttggagaatactgccaaaatgtgtatgacaagctgtattacggc | 38 |
| | | ttttgtttatattttttttaac | |
| Pdgfrb | F | Gggacagcttgtggaagcgttgctgctgggaggcc | 39 |
| site | R | Ggcctcccagcagcaacgcttccacaagctgtccc | 40 |
| mut | | | |

Mutagenized targets are underlined.

TABLE 6
Primary antibodies.

| | | WB | IHC |
|---|---|---|---|
| Antibody | Manufacturer | dilution | dilution |
| anti-ACTB | Novus Biological | 1/5,000 | — |
| | (NB600-501) | | |
| anti-AKT | Cell Signaling Technologies | 1/1,000 | — |
| | (#9272) | | |
| anti-FOXO3 | Abcam | 1/1,000 | 1/500 |
| | (ab12162) | | |
| anti-GAPDH | Santa Cruz Biotech | 1/5,000 | — |
| | (sc-32233) | | |
| anti-JAK1 | Cell Signaling Technologies | 1/500 | — |
| | (#3334) | | |
| anti-P115 | Kindly provided by the De | 1/5,000 | — |
| | Matteis Lab | | |
| anti-p-AKT (Ser⁴⁷³) | Cell Signaling Technologies | 1/1,000 | — |
| | (#4058) | | |
| anti-PDGFRα | Cell Signaling Technologies | 1/500 | — |
| | (#3174) | | |
| anti-PDGFRβ | Cell Signaling Technologies | 1/500 | — |
| | (#3169) | | |
| anti-p-FOXO3 (Ser⁵⁷⁴) | Signalway Antibody | 1/250 | — |
| (used for mouse | (12874) | | |
| samples) | | | |
| anti-p-FOXO3 (Ser⁵⁷⁴) | Kindly provided by the | 1/500 | — |
| (used for human | Weiman's Lab | | |
| samples) | | | |
| anti-p-JAK1 | Cell Signaling Technologies | 1/500 | — |
| (Tyr¹⁰³⁴/Tyr¹⁰³⁵) | (#3331) | | |
| anti-p-PDGFRα/β | Cell Signaling Technologies | 1/500 | — |
| (Tyr⁸⁴⁹/Tyr⁸⁵⁷) | (#3170) | | |
| anti-p-SAPK/JNK | Cell Signaling Technologies | 1/1,000 | — |
| (Thr¹⁸³/Tyr¹⁸⁵) | (#9251) | | |
| anti-RAD50 | Santa Cruz Biotech | 1/250 | — |
| | (sc-74460) | | |
| anti-SAPK/JNK | Cell Signaling Technologies | 1/1,000 | — |
| | (#9252) | | |
| Anti-histone H3 | Abcam | 1/5,000 | — |
| | (ab4441) | | |

WB: Western blot;
IHC: immunohistochemistry.

TABLE 7
Upregulated genes in PiZ/miR-34b/c^{-/-} versus PiZ/miR-34b/c^{+/+} that
are predicted to be targeted by miR-34b/c.

| EnsemblGeneID | Symbol | logFC_EXP2vsEXP1 | FDR_EXP2vsEXP1 |
|---|---|---|---|
| ENSMUSG00000054611 | Kdm2a | 0.313 | 0.034957823 |
| ENSMUSG00000017291 | Taok1 | 0.33 | 0.00881375 |
| ENSMUSG00000003617 | Cp | 0.34 | 0.018000003 |
| ENSMUSG00000048833 | Slc39a9 | 0.354 | 0.037692369 |
| ENSMUSG00000049606 | Zfp644 | 0.379 | 0.021717669 |
| ENSMUSG00000028557 | Rnf11 | 0.4 | 0.018831342 |
| ENSMUSG00000022443 | Myh9 | 0.44 | 0.003186945 |
| ENSMUSG00000025255 | Zfhx4 | 0.44 | 0.039832073 |
| ENSMUSG00000026827 | Gpd2 | 0.456 | 0.010722766 |
| ENSMUSG00000074305 | Peak1 | 0.459 | 0.019917834 |
| ENSMUSG00000039176 | Polg | 0.464 | 0.037283144 |
| ENSMUSG00000040479 | Dgkz | 0.481 | 0.04717876 |
| ENSMUSG00000001525 | Tubb5 | 0.492 | 0.035661548 |
| ENSMUSG00000024614 | Tmx3 | 0.497 | 0.023633012 |
| ENSMUSG00000026594 | Ralgps2 | 0.515 | 0.013026839 |
| ENSMUSG00000040274 | Cdk6 | 0.53 | 0.00138706 |
| ENSMUSG00000055065 | Ddx17 | 0.533 | 0.007537239 |
| ENSMUSG00000031217 | Efnb1 | 0.535 | 0.004918379 |
| ENSMUSG00000032393 | Dpp8 | 0.563 | 0.045591847 |
| ENSMUSG00000037270 | 4932438A13Rik | 0.584 | 0.000407238 |
| ENSMUSG00000025790 | Slco3a1 | 0.61 | 0.037186636 |
| ENSMUSG00000027087 | Itgav | 0.655 | 0.030527502 |
| ENSMUSG00000005583 | Mef2c | 0.681 | 0.021017252 |
| ENSMUSG00000013698 | Pea15a | 0.696 | 0.013492371 |
| ENSMUSG00000038351 | Sgsm2 | 0.717 | 0.020701638 |
| ENSMUSG00000039834 | Zfp335 | 0.736 | 0.024144788 |
| ENSMUSG00000069662 | Marcks | 0.749 | 0.000279862 |
| ENSMUSG00000038178 | Slc43a2 | 0.777 | 0.004628457 |
| ENSMUSG00000021493 | Pdlim7 | 0.833 | 0.016601083 |
| ENSMUSG00000042446 | Zmym4 | 0.841 | 0.027712393 |
| ENSMUSG00000040209 | Zfp704 | 0.867 | 0.013986987 |
| ENSMUSG00000035566 | Pcdh17 | 0.869 | 0.035551529 |
| ENSMUSG00000025666 | Tmem47 | 0.881 | 0.02858255 |
| ENSMUSG00000026657 | Frmd4a | 0.902 | 0.002231081 |
| ENSMUSG00000022636 | Alcam | 0.965 | 4.35366E−11 |
| ENSMUSG00000022883 | Robo1 | 0.988 | 0.033546169 |
| ENSMUSG00000026821 | Ralgds | 1.004 | 0.041470799 |
| ENSMUSG00000038679 | Trps1 | 1.047 | 0.02144072 |
| ENSMUSG00000020427 | Igfbp3 | 1.058 | 0.029838814 |
| ENSMUSG00000039145 | Camk1d | 1.083 | 0.000111332 |
| ENSMUSG00000028073 | Pear1 | 1.166 | 0.018068822 |
| ENSMUSG00000021186 | Fbln5 | 1.167 | 0.030970872 |
| ENSMUSG00000045287 | Rtn4rl1 | 1.261 | 0.001487532 |
| ENSMUSG00000025821 | Zfp282 | 1.295 | 6.9356E−05 |
| ENSMUSG00000029231 | Pdgfra | 1.419 | 0.001095503 |
| ENSMUSG00000031343 | Gabra3 | 1.436 | 0.000512361 |
| ENSMUSG00000011884 | Gltp | 1.469 | 0.000933098 |
| ENSMUSG00000014303 | Glis2 | 1.54 | 0.043576876 |
| ENSMUSG00000025577 | Cbx2 | 1.552 | 0.000450612 |
| ENSMUSG00000006586 | Runx1t1 | 1.579 | 0.005734921 |
| ENSMUSG00000024620 | Pdgfrb | 1.698 | 0.000195207 |
| ENSMUSG00000044734 | Serpinb1a | 2.158 | 1.89314E−05 |
| ENSMUSG00000044646 | Zbtb7c | 2.409 | 0.013002945 |
| ENSMUSG00000076431 | Sox4 | 2.418 | 5.14055E−21 |
| ENSMUSG00000002020 | Ltbp2 | 2.623 | 0.00366541 |
| ENSMUSG00000003352 | Cacnb3 | 2.665 | 0.00145797 |
| ENSMUSG00000055254 | Ntrk2 | 2.759 | 0.022454347 |
| ENSMUSG00000027737 | Slc7a11 | 3.628 | 9.47175E−07 |

### EXAMPLE 1

### Materials and Methods

### Mouse Studies

Male 6- to 8-week-old C57BL/6 (Charles River Laboratories) and miR-34b/c^{-/- 16} (Jackson laboratory) mice were used. TAA (Sigma-Aldrich) was dissolved in phosphate buffered saline (PBS) and administered by intraperitoneal injection three times a week for four weeks with escalating doses, starting from 50 mg/kg/day to 200 mg/kg/day, as previously described ¹⁷. CCl₄ (Sigma-Aldrich) was dissolved in corn oil (Sigma-Aldrich) and administered by gavage three times a week for four weeks with escalating doses, starting from 0.875 ml/kg/day to 2.5 ml/kg/day, as previously described ¹⁷. At sacrifice, mice were perfused with PBS.

Murine Mir34b and Mir34c regions were PCR amplified from genomic DNA of C57BL/6 mice using the following primers Mir34b- rev 5′-CGCGGATCCTTGCGGG AAGAAGGACTCG-3′ (SEQ ID NO:41), Mir34b-fw 5′-ATTTGCGGCCGCTCCGAGGGTTACTTGCACTTA-3′(SEQ ID NO:42), Mir34c-fw 5′-GCGGCCGCAGTCAATATAATGACCAAATCAGCTAAG-3′ (SEQ ID NO:43), Mir34c-rev 5′-GGATCCCAGAACAGTTCCTGCTGCTG-3′ (SEQ ID NO:44). Amplified Mir34b and Mir34c were cloned in an AAV2.1 plasmid including the TBG promoter. Serotype 8 AAV vectors were produced by triple transfection of HEK293 cells, as previously described ¹⁸. AAV vectors were injected intravenously in the retro-orbital venous plexus in a volume of 100 μl at a total dose of 1×10¹³ genome copies/Kg.

Sirius Red staining was performed on 5-μm liver sections which were rehydrated and stained for 1 hour in picrosirius red solution (0.1% Sirius red in saturated aqueous solution of picric acid). After two changes of acidified water (0.5% acetic acid in water), sections were dehydrated, cleared in xylene, and mounted in a resinous medium. Images were captured by Axio Scan.Z1 microscope (Zeiss) and analyzed by ImageJ for quantification of Sirius Red positive area. Five images for each mouse were analyzed. Sections were analyzed blinded by an experienced pathologist (S.C.) for fibrosis staging using Ishak scoring system ¹⁹.

For gene expression analyses, total RNA from cells and livers was extracted using RNeasy mini kit (QIAGEN). 1-2 μg of RNA were retro-transcribed using High-Capacity cDNA Reverse Transcription Kit (Applied Biosystems). The qPCR reactions were set up using SYBR Green Master Mix and run in duplicate on a Light Cycler 480 system (Roche). Primers are reported in Table 1. Running program was as follows: pre-heating, 5 minutes at 95° C.; 40 cycles of 15 seconds at 95° C., 15 seconds at 60° C., and 25 seconds at 72° C. B2m and B2M were used as housekeeping genes. Data were analyzed using LightCycler 480 software version 1.5 (Roche).

For immunohistochemistry, 5-μm thick sections were rehydrated and antigen unmasking was performed in 0.01 M citrate buffer in a microwave oven. Next, sections underwent blocking of endogenous peroxidase activity in methanol/1.5% H₂O₂ (Sigma-Aldrich) for 30 min and were incubated with blocking solution (3% bovine serum albumin [Sigma-Aldrich], 5% donkey serum [Millipore], 1.5% normal goat serum [Vector Laboratories] 20 mM MgCl₂, 0.3% Triton [Sigma-Aldrich] in PBS) for 1 h. Sections were incubated with primary antibody anti-Collagen type I (Table 2) overnight at 4° C. and then with universal biotinylated goat anti-rabbit IgG secondary antibody (Vector Laboratories) for 1 h. Biotin/avidin-horseradish peroxidase (HRP) signal amplification was achieved using ABC Elite Kit (Vector Laboratories) according to manufacturer's instructions. 3,3′-diaminobenzidine (Vector Laboratories) was used as peroxidase substrate. Mayer's hematoxylin (Bio-Optica) was used for counter-staining. Sections were de-hydrated and mounted in mounting medium (Leica Biosystems).

Hepatic hydroxyproline content was measured as previously described¹⁷. Briefly, homogenized liver tissue was hydrolyzed in 6N HCl at 110° C. for 16 h. Hydrolysates were filtered and assayed in citrate-acetate buffer. Samples were incubated with Chloramine-T solution (Sigma-Aldrich) for 20 min at RT. Next, Ehrich's reagent (Sigma-Aldrich) was added, samples were incubated at 65° C. for 20 min, and absorbance was measured at 550 nm.

For Western blotting, proteins from tissues were extracted in RIPA buffer according to standard procedures. Primary antibodies were diluted in TBS-T/5% milk (Bio-Rad Laboratories) (Table 2). Secondary antibodies were enhanced chemiluminescence (ECL) anti-rabbit HRP and ECL anti-mouse HRP (GE Healthcare). Peroxidase substrate was provided by ECL Western Blotting Substrate kit (Pierce). Analysis of band intensities was performed using Quantity One 1-D Analysis Software version 4.6.7 (Bio-Rad Laboratories).

Hepatic levels of mmu-miR34b-5p and mmu-miR-34c-5p were analyzed as previously described²⁰.

### Cell Studies

LX-2 cells and Huh-7 cells were maintained at 37° C. in a humidified atmosphere of CO₂ and were co-cultured in a 1:5 ratio (LX2 to Huh-7) in Dulbecco's Modified Eagle medium (DMEM) supplemented with 2% and 10%, respectively, of fetal bovine serum, plus 1% penicillin/streptomycin and 1% of glutamine. The day after seeding, cells were incubated with 2 ng/ml of human TGF-β1 (Sigma Aldrich). After 24 hours, cells were transfected with 100 nM of miRIDIAN microRNA mimic negative control, with 50nM each of miRIDIAN mimic hsa-miR34b-5p and hsa-miR-34c-5p (Dharmacon), or with transfection reagent only. Interferin transfection reagent (Polyplus) was used for transfection according to the manufacturer's instructions. Cells were harvested 48 hours after transfection.

To validate COL1A1 as miR-34b/c targets, human COL1A1 3′-UTR was amplified by PCR from human genomic DNA using the following primers: 3′-UTR-fw 5′-GCTCGCTAGCCTCGAGACTCCCTCCATCCCAACC-3′ (SEQ ID NO:45) and 3′-UTR-rev 5′- ATGCCTGCAGGTCGACAAGCTTAAAAAGGAGTAGGCGGG-3′ (SEQ ID NO:46). PCR product was cloned downstream the firefly luciferase gene into pmirGLO Dual-Luciferase miRNA Target Expression plasmid (Promega). The miR-34b/c 7-mer recognition site was mutagenized by QuickChange site-directed mutagenesis kit (Agilent) according to manufacturer's instructions. Primers used for mutagenesis were the follows (mutagenized nucleotides are 5′- underlined): 3′-UTR mut-5′-CCCGCCCCCCGGTAGCTGCCCCGGTGACACATC-3′ (SEQ ID NO:47) and 3′-UTR mut-rev 5′-GATGTGTCACCGGGGCAGCTACCGGGGGGCGGG-3′ (SEQ ID NO:48). HeLa cells cultured in DMEM plus 10% FBS and 5% penicillin/streptomycin were co-transfected with the plasmid containing the wild-type or mutagenized COL1A1 3′-UTR and with negative control, miRIDIAN mimic has-miR-34b-5p or has-miR-34c-5p (Dharmacon) using Interferin transfection reagent (Polyplus). Cells were harvested 72 h after transfection and assayed for luciferase activity by the Dual-Luciferase Reporter Assay System (Promega). Data were expressed relative to renilla luciferase activity to normalize for transfection efficiency. Firefly-to-renilla activity ratio for each triplicate was normalized to the average of negative control transfected samples.

For Western blotting, proteins from tissues were extracted in RIPA buffer according to standard procedures. Primary antibodies were diluted in TBS-T/5% milk (Bio-Rad Laboratories) (Table 2). Secondary antibodies were ECL anti-rabbit HRP and ECL anti-mouse HRP (GE Healthcare). Peroxidase substrate was provided by ECL Western Blotting Substrate kit (Pierce). Analysis of band intensities was performed using Quantity One 1-D Analysis Software version 4.6.7 (Bio-Rad Laboratories).

### RNA-Seq

For RNA-seq analysis, library preparation was performed with a total of 100 ng of RNA from each sample using QuantSeq 3′mRNA-Seq Library prep kit (Lexogen) according to manufacturer's instructions. Amplified fragmented cDNA of 300 bp in size was sequenced in single-end mode by NovaSeq 6000 (Illumina) with a read length of 100 bp. Illumina NovaSeq 6000 base call (BCL) files were converted in fastq file through bcl2fastq. Sequence reads were trimmed BBDuk (sourceforge.net/projects/bbmap/) to remove adapter sequences and low-quality end bases (Q<20). Alignment was performed with STAR 2.6.0a²¹ on the Hg38 reference provided by UCSC Genome Browser ²². Gene expression levels were determined with HTseq-count 0.9.1²³. The raw expression data were normalized, analyzed and visualized by Rosalind HyperScale architecture (OnRamp BioInformatics, Inc.)²⁴. False Discovery Rate (FDR) <0.05 was considered as statistically significant. Data were deposited in GEO with the accession number GSE179200. GSEA was performed using the GSEA software (www.broadinstitute.org/gsea)²⁵ and restricting the input to the three gene lists (ACTIVATED HSC signature²⁶; QUIESCENT HSC signature²⁶ and REACTOME_COLLAGEN_FORMATION). FDR<0.25 was considered as statistically significant. Gene Ontology Enrichment Analysis (GOEA) was performed on the 71 genes regulated in opposite correlation (Dataset S5) by using the DAVID online tool (DAVID Bioinformatics Resources 6.8²⁷) restricting the output to Biological Process terms (BP_FAT) and Cellular Compartment terms (CC_FAT). Putative miR-34b/c target genes were identified by DIANA-microT-CDS software^{28,29}.

### Statistical Analyses

One- or two-way ANOVA plus Tukey's post-hoc or Kurskal-Wallis plus Dunn's multiple comparison were used as statistical tests. Statistical analysis used for each experiment are reported in figure legends. Experimental group sizes are reported in figure legends Data are reported as average ±standard error.

### Results

Deletion of miR-34b/c Increases Liver Fibrosis

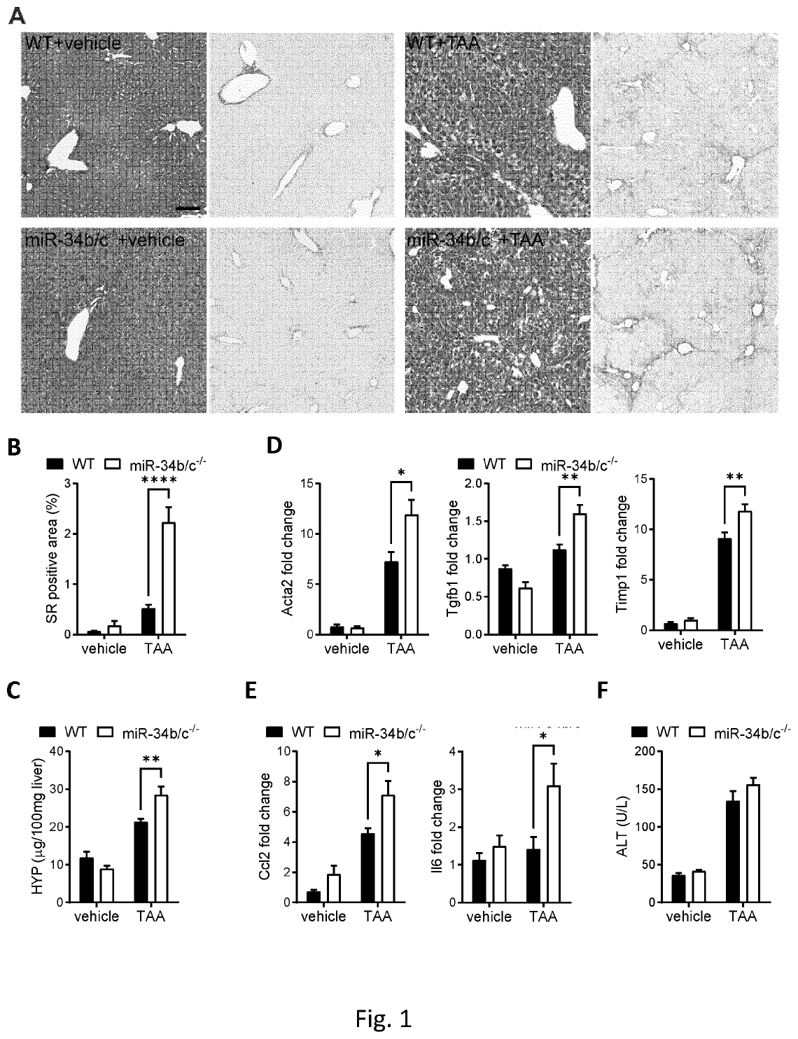

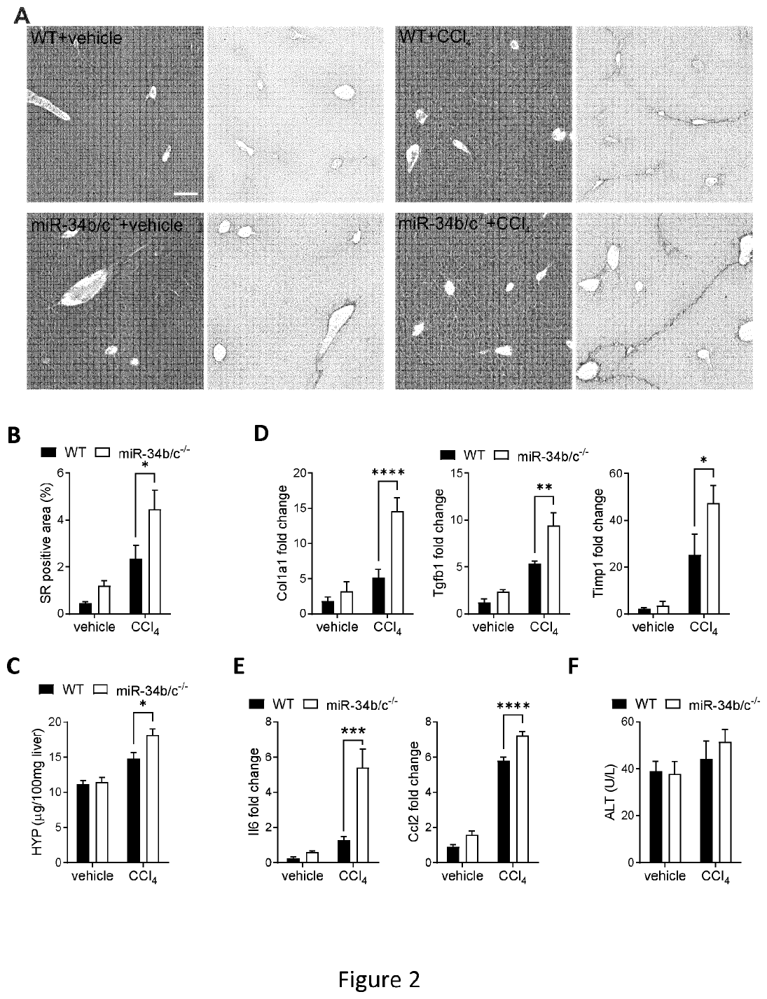

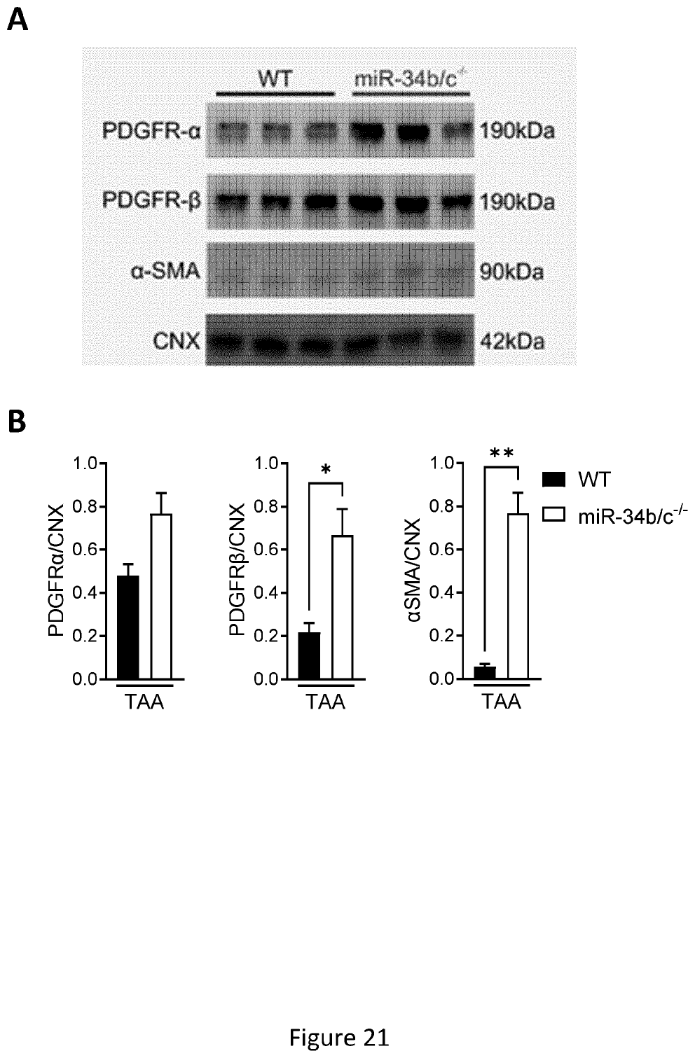

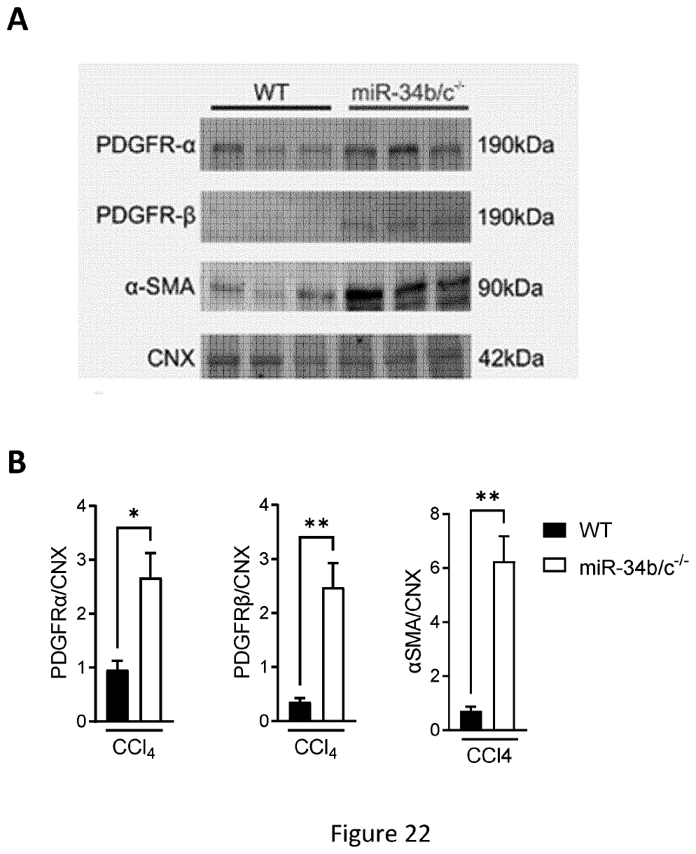

To investigate the role of miR-34b/c in liver fibrosis, inventors treated miR-34b/c^{-/-} and wild-type control mice with multiple types of pro-fibrotic insults. First, mice were treated with increasing doses of thioacetamide (TAA) or carbon tetrachloride (CCl₄) for 4 and 6 weeks, respectively or with vehicles as controls. TAA treatment induced liver fibrosis in both miR-34b/c^{-/-} and wild-type control mice (FIG. 1A) but livers from TAA-treated miR-34b/c^{-/-} mice showed significantly larger Sirius red (SR)-positive area and higher hydroxyproline content compared to TAA-treated wild-type mice (FIGS. 1A-C). Moreover, expression of fibrosis and inflammatory genes was greater in miR-34b/c^{-/-} compared to wild-type mice in response to TAA treatment (FIGS. 1D,E). Consistently, increased α and β subunits of PDGF receptor (PDGFR-α and -β), that are directly targeted by miR-34b/c ³⁰, and α-smooth muscle actin (α-SMA) proteins that are markers of HSC activation, were detected in TAA-treated miR-34b/c^{-/-} mouse livers compared to wild-type (FIG. 21). Alanine transaminase (ALT) serum levels were increased by TAA treatment to similar levels in wild-type and miR-34b/c^{-/-} compared to vehicle-treated controls (FIG. 1F). Similarly, after treatment with CCl₄, miR-34b/c^{-/-} livers showed increased SR staining and hydroxyproline content (FIGS. 2A-C), upregulation of fibrosis and inflammatory genes (FIGS. 2D,E) and increased PDGFR-α/β and a-SMA proteins (FIG. 22). compared to control livers. These data support a protective role against liver fibrosis of miR-34b/c, CCl₄ treatment resulted in small to no increase serum ALT compared to vehicle-treated animals (FIG. 2F), which was consistent with previous studies using similar treatment schedule and dosing ^{5.31}. Along with the data showing that mice expressing the mutant Z α1-antitrypsin have greater liver fibrosis (vide infra), these data support a protective role of miR-34b/c against liver fibrosis.

miR-34b/c Antagonizes Human Hepatic Stellate Cells Activation

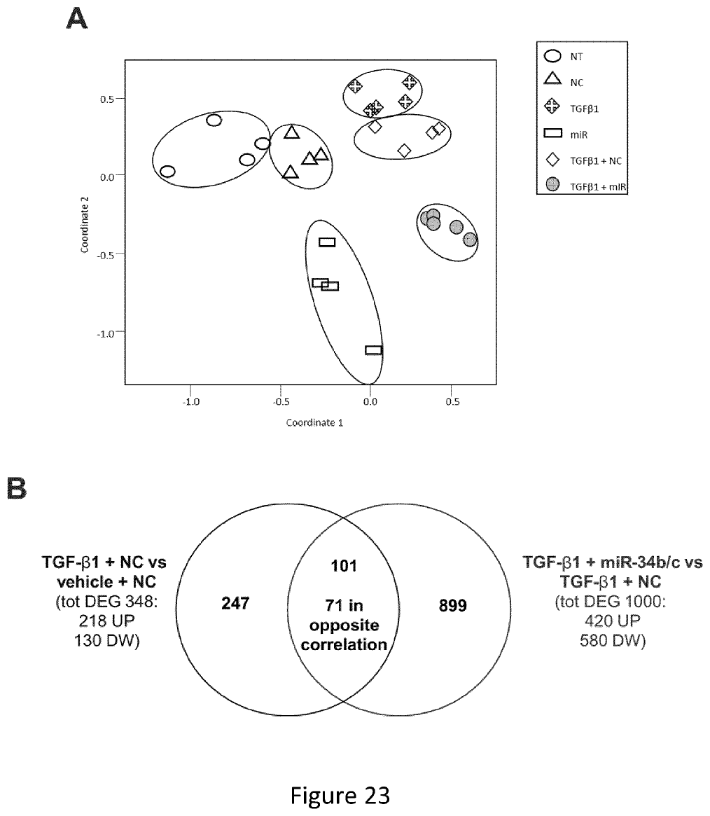

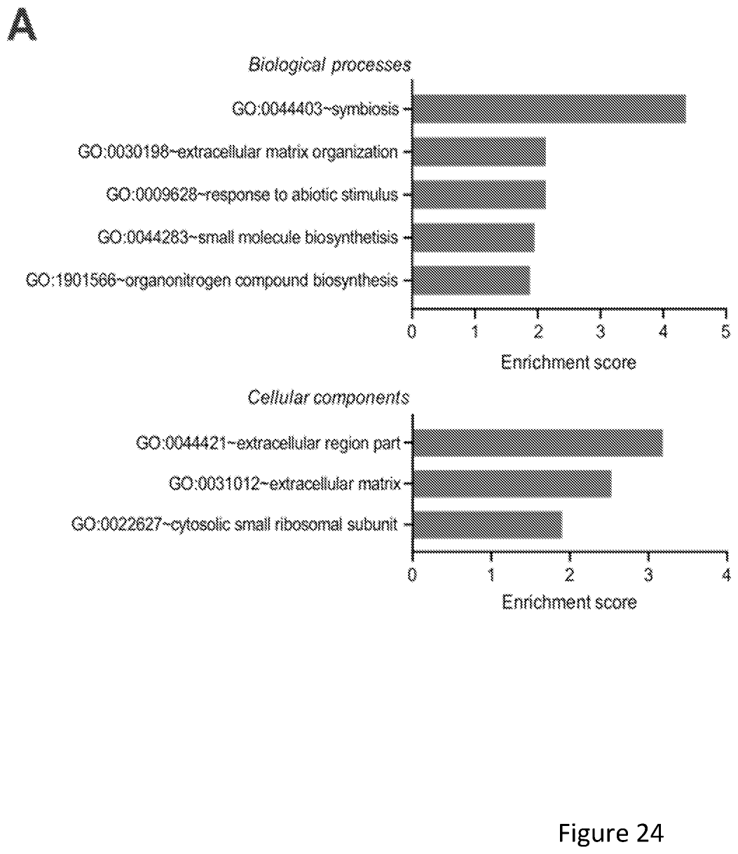

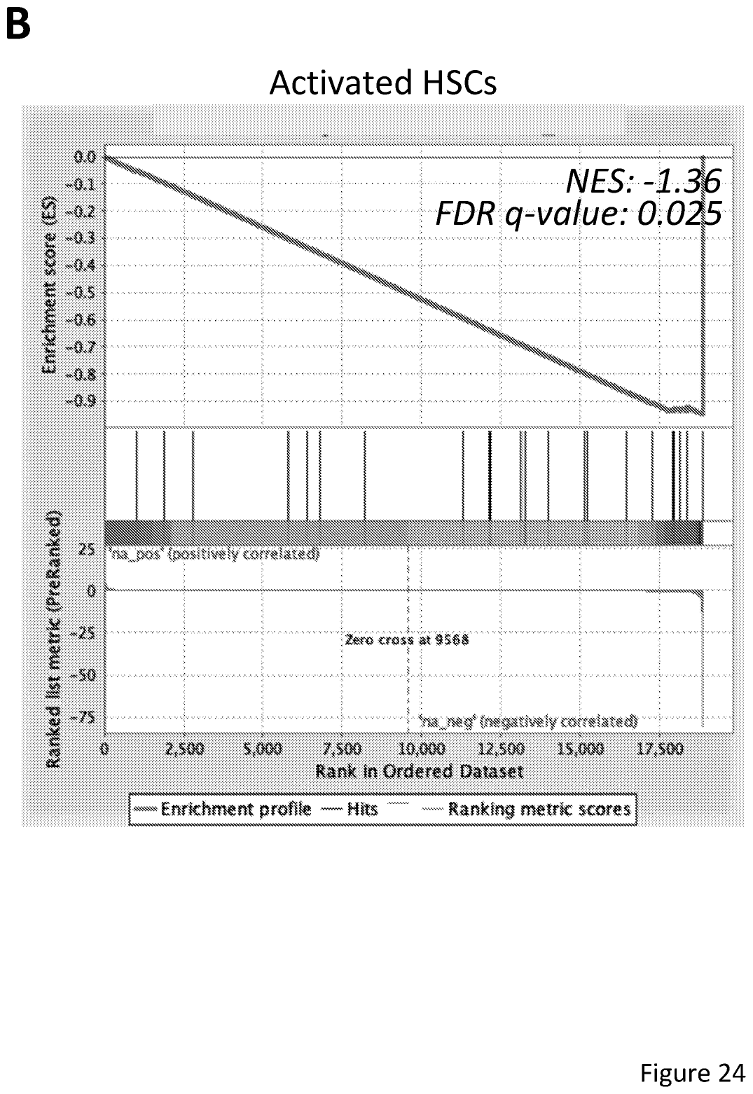

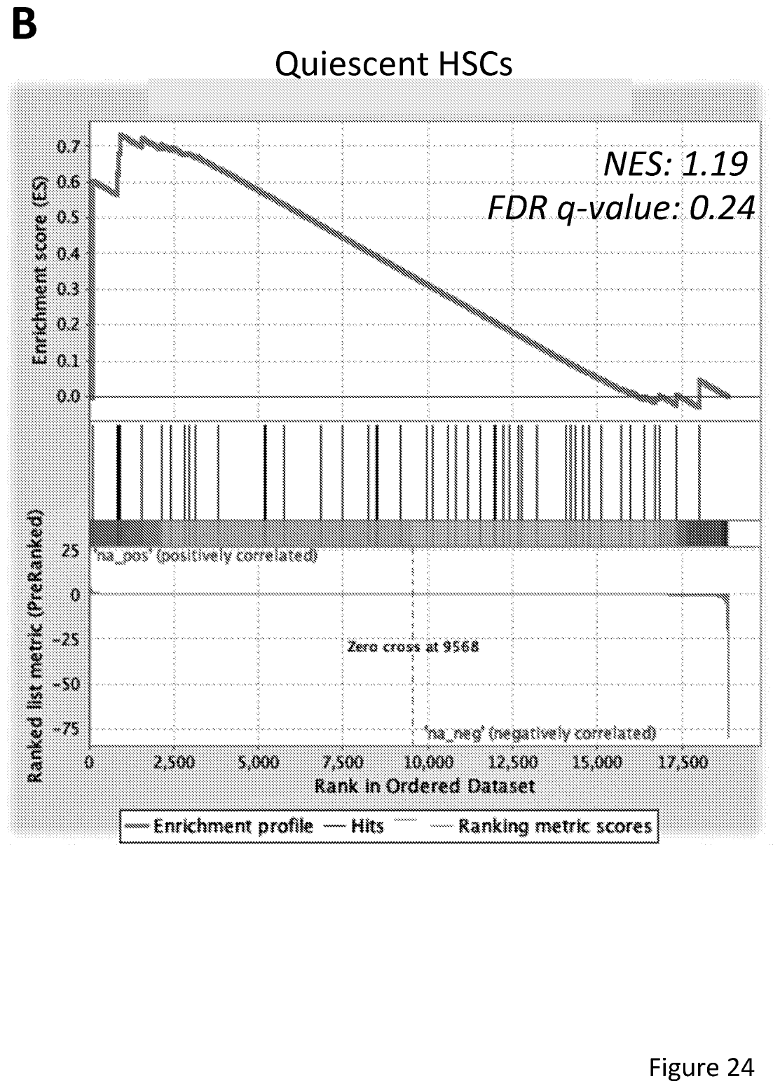

miR-34b/c belongs to a family of evolutionarily conserved miRNAs³². To investigate whether its anti-fibrotic activity is conserved in humans, the investigators transfected LX2 cells, a human hepatic stellate cell (HSC) line, with the miR-34b and miR-34c mimics. As controls, LX2 cells were transfected with a negative control mimic or were left untreated. All experimental groups included cells under either normal culturing condition or incubated with human TGF-β1 to induce HSC activation. By transcriptomic analysis, un-transfected and negative control transfected cells showed similar expression profiles (FIG. 23A). In contrast, compared to the negative control, cells transfected with miR-34b/c showed significant transcriptional changes (FIG. 23A) with 606 differentially expressed genes (280 up- and 326 downregulated) under basal conditions and 1,000 differentially expressed genes (420 up- and 580 downregulated) after TGF-β1 treatment (see GSE179200, Gene Expression Omnibus database). In negative control cells, TGF-β1 treatment resulted in 348 differentially expressed genes and 71 of these genes showed opposite correlation in TGF-β1-treated and miR-34b/c-transfected cells compared to negative control cells (FIG. 23B). Functional annotation clustering analysis of the 71 genes with opposite correlation revealed an enrichment of genes encoding components of the extracellular matrix or genes involved in its processing (FIG. 24A). Moreover, by gene set enrichment analysis (GSEA) based on HSC gene signatures26, compared to negative control cells, miR-34b/c-transfected TGF-β1-treated LX2 cells showed enrichment of genes of the activation signature that were downregulated and enrichment of genes of the quiescence signature that were upregulated (FIG. 24B).

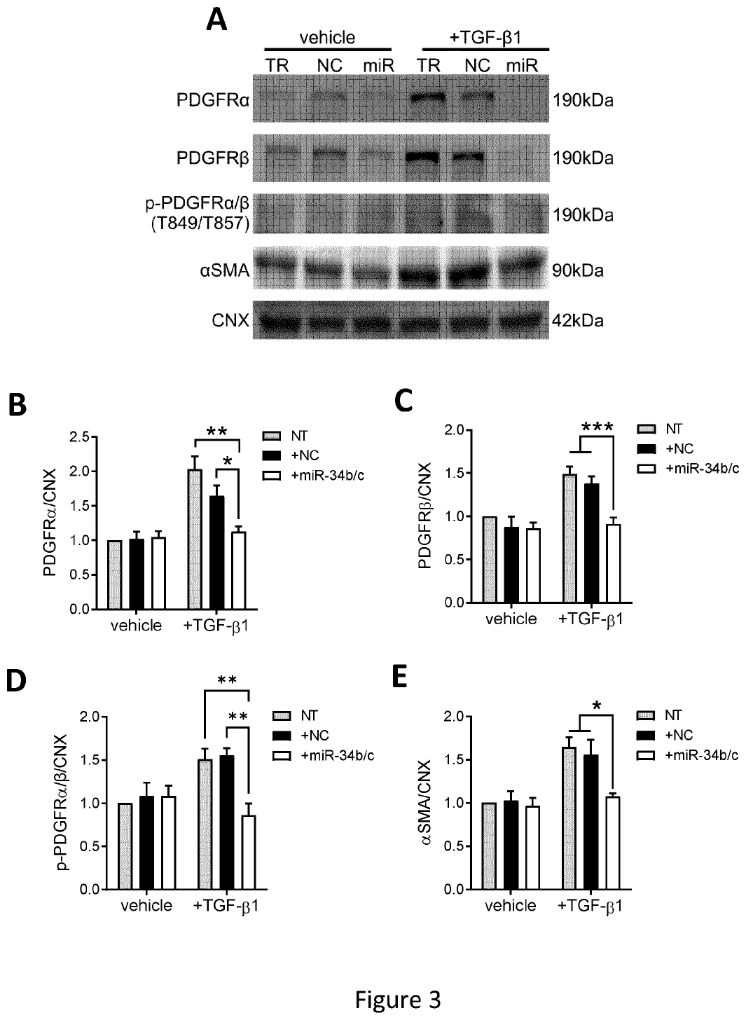

To further investigate miR-34b/c anti-fibrotic activity in humans inventors co-cultured hepatocytes (Huh7 cells) and hepatic stellate cell (LX-2) lines and transfected them with miR-34b/c mimics or a negative control mimic after incubation with the profibrogenic human Transforming Growth Factor-β1 (TGF-β1). As expected, TGF-β1 increased expression and phosphorylation of both α and β subunits of PDGF receptor (PDGFRα and β) and a-SMA, indicating HSC activation. Compared to the negative controls, miR-34b/c mimic significantly reduced PDGFRa and B, that are directly targeted by miR-34b/c ³⁰, (FIG. 3A-C), PDGFRα/β phosphorylation and α-SMA levels (FIG. 3A,D,E). Taken together, these findings support miR-34b/c inhibition of TGF-β1-induced HSC activation in the LX2 cell model.

miR-34b/c Directly Inhibits Expression of Genes Involved in Collagen Biosynthesis

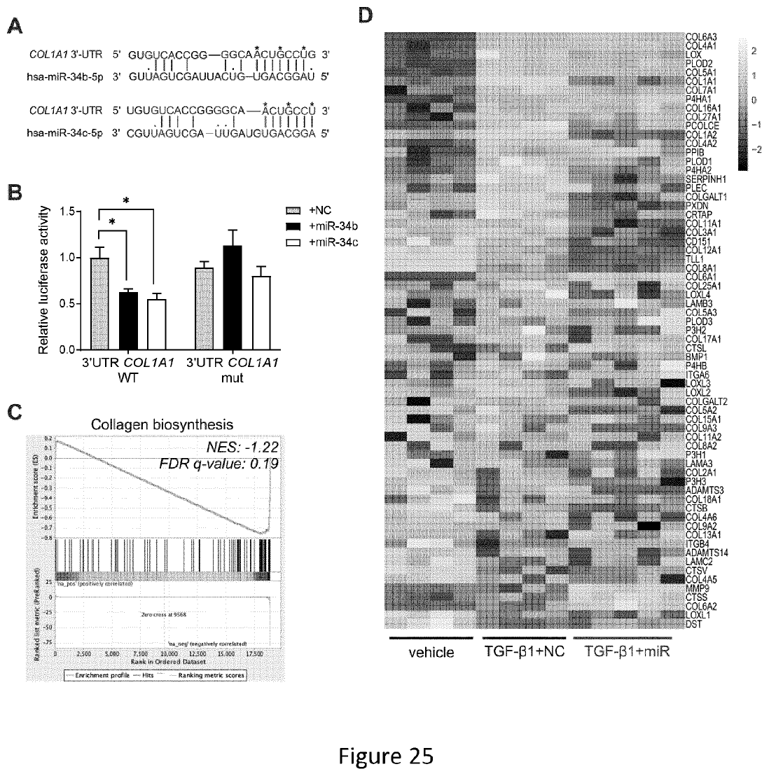

Type I collagen is strongly induced by pro-fibrotic stimuli and is the most abundant component of fibrotic liver scars33. By RNA-seq analysis, COL1A1 and COL1A2 genes encoding the pro-α-1 and pro-α-2 chains of type I collagen were downregulated after miR-34b/c transfection (GSE179200, Gene Expression Omnibus database). Interestingly, COL1A1 was retrieved as a target of miR-34c by a genome-wide analysis of miRNA-mRNA interactions in human mesenchymal stem cells34. Consistently, COL1Al3′-untranslated region (3′-UTR) includes two putative target sites for miR-34c (one 7-mer and one 6-mer) (FIG. 25A). When the 7-mer site located at the 3′-UTR of the luciferase gene was mutagenized, miR-34b and -34c failed to suppress luciferase activity (FIG. 25B). By GSEA, genes involved in collagen biosynthesis showed significant enrichment among downregulated genes in TGF-β1-treated LX2 cells transfected with the miR-34b/c mimics, compared to cells transfected with the negative control (FIGS. 25C,D). Notably, several key genes involved in collagen maturation and deposition contains putative miR-34b/c target sites and their downregulation or deletion has been previously associated with protection against liver fibrosis.

Hepatic-Specific Delivery of miR-34b/c Attenuates Liver Fibrosis

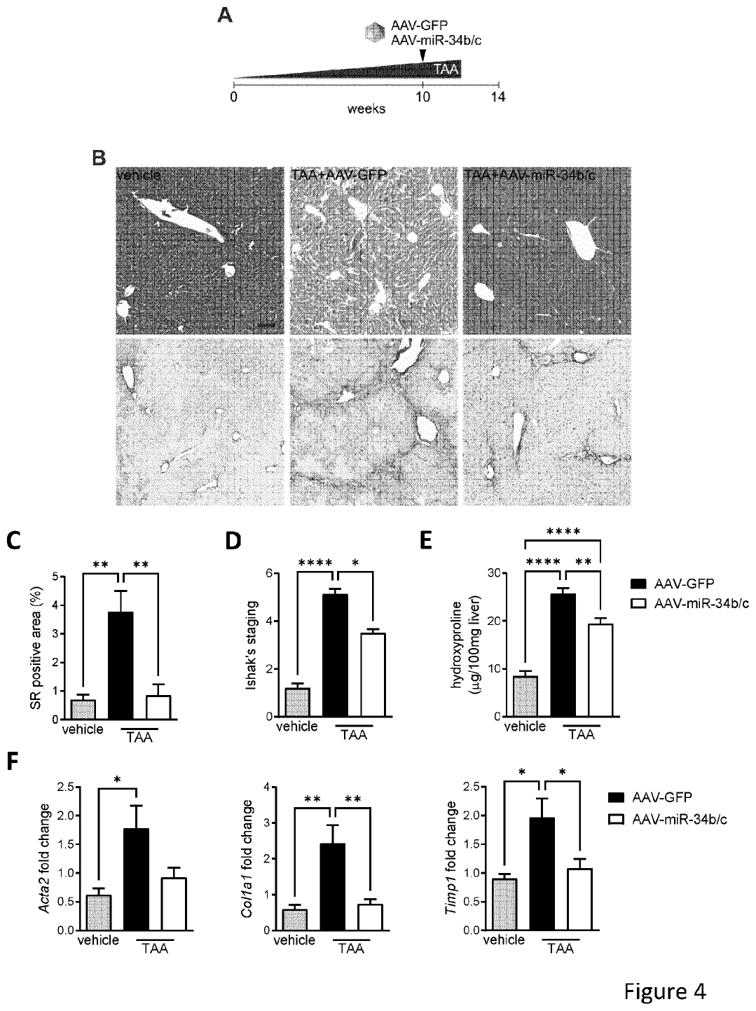

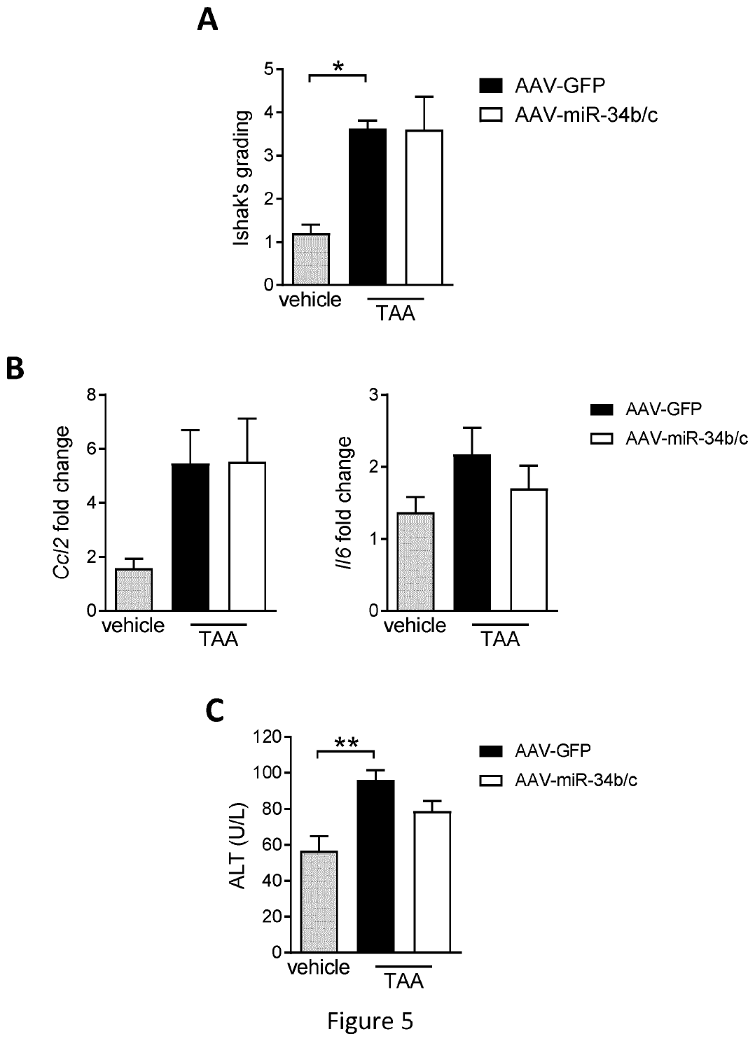

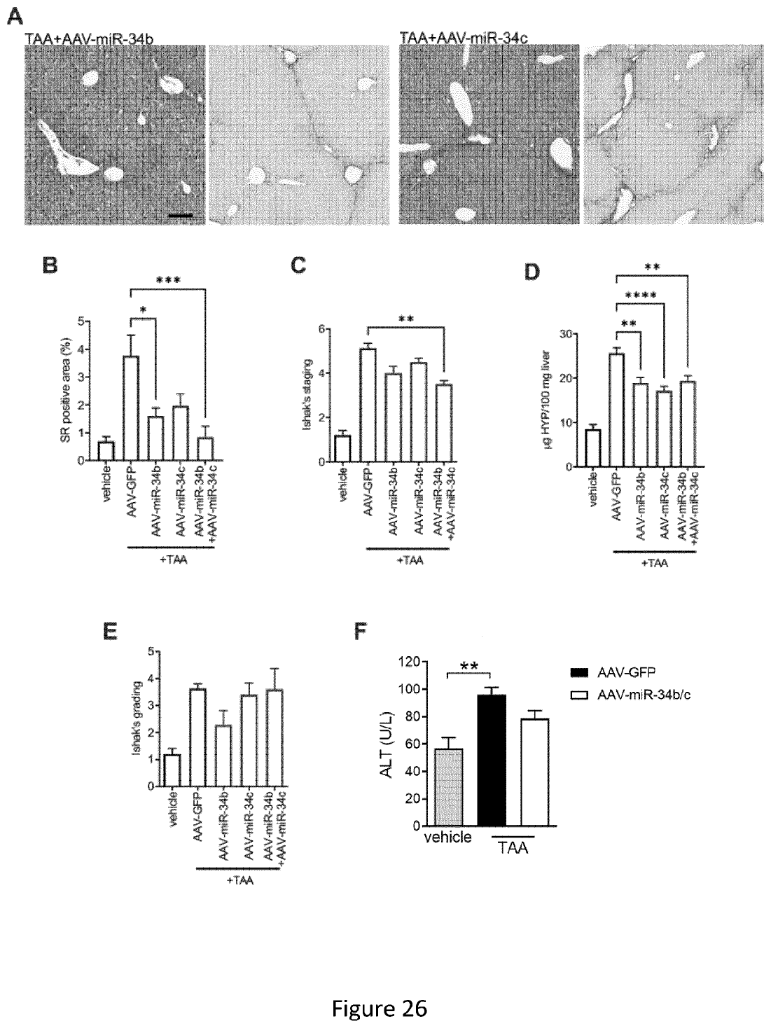

Inventors hypothesize that hepatocyte-specific delivery of miR-34b/c reduces liver fibrosis. To investigate this, inventors generated serotype 8 adeno-associated vectors (AAV) expressing murine miR-34b or miR-34c under the control of hepatocyte-specific thyroxine binding protein promoter. Wild-type mice were treated for 12 weeks with increasing doses of TAA or vehicle to induce advanced fibrosis and cirrhosis ¹⁷. After 10 weeks of TAA treatment, mice were intravenously injected with AAV-miR-34b, AAV-miR-34c, AAV-miR-34b/c or an AAV expressing green fluorescent protein (GFP) as controls and sacrificed 4 weeks later (FIG. 4A). TAA induced bridging fibrosis to cirrhosis in livers of AAV-GFP injected mice (FIG. 4B-D). Compared to AAV-GFP injected mice, livers of AAV-miR-34b/c injected mice showed significantly reduced fibrosis (FIG. 4B-D), consistent with lower hepatic hydroxyproline content (FIG. 4E). Moreover, livers of AAV-miR-34b/c injected mice showed normalized expression of fibrosis marker gene Acta2, Col1a1 and Timp1 (FIG. 4F). Livers of mice injected with AAV-miR-34b alone also showed reduction in SR positive area and hydroxyproline content and a trend in reduction of Ishak's fibrosis score, whereas livers of mice injected with AAV-miR-34c only showed significant reduction of hydroxyproline amount compared to AAV-GFP injected mice (FIGS. 26A-E). Grading of necro-inflammatory activity showed significant increase in TAA-treated compared to vehicle-treated mice, but no significant changes were detected between AAV-miR-34b/c and AAV-GFP injected animals (FIG. 5A). Expression of inflammatory genes Il6 and Ccl2 showed no significant differences between vehicle- and TAA-treated animals (FIG. 5B), possibly as a consequence of prolonged treatment ³⁵. Serum ALT levels were significantly increased by TAA-treatment, while a small, non-significant reduction was observed in AAV-miR-34b/c- compared to AAV-GFP-injected animals (FIG. 5C).

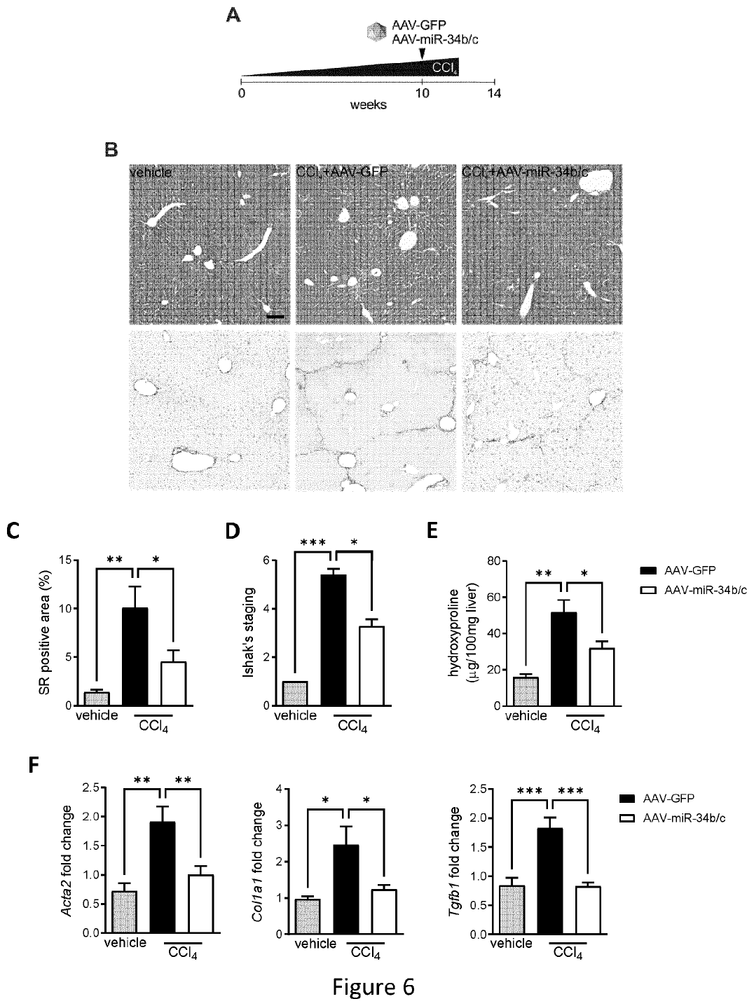

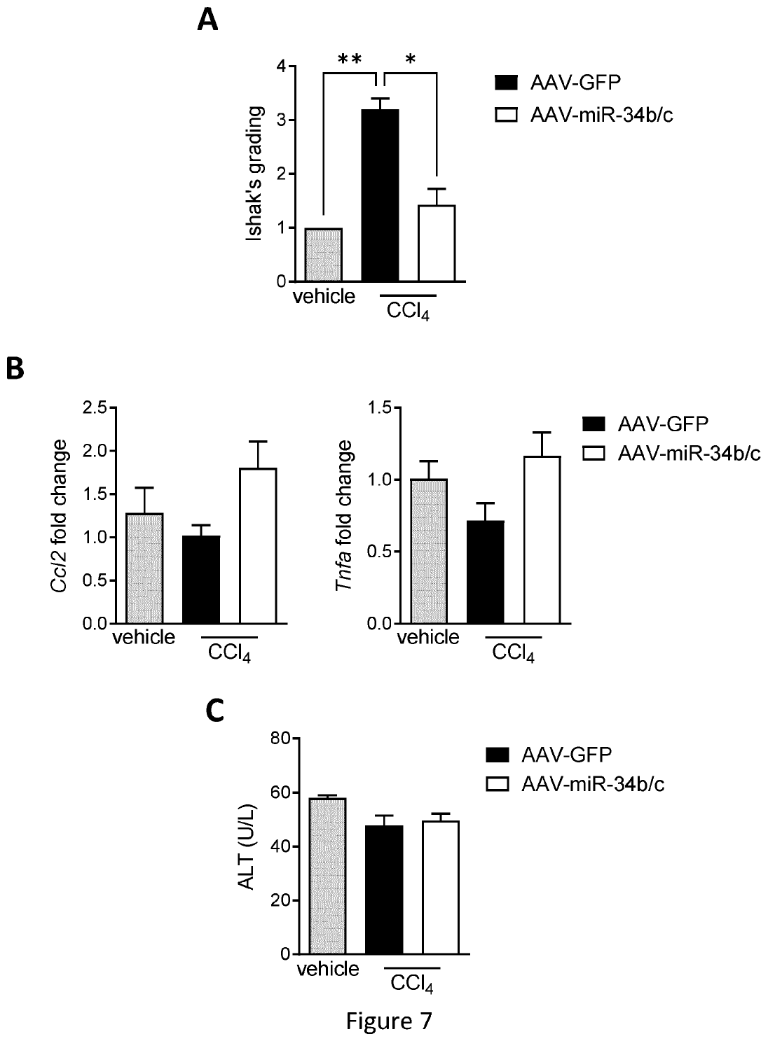

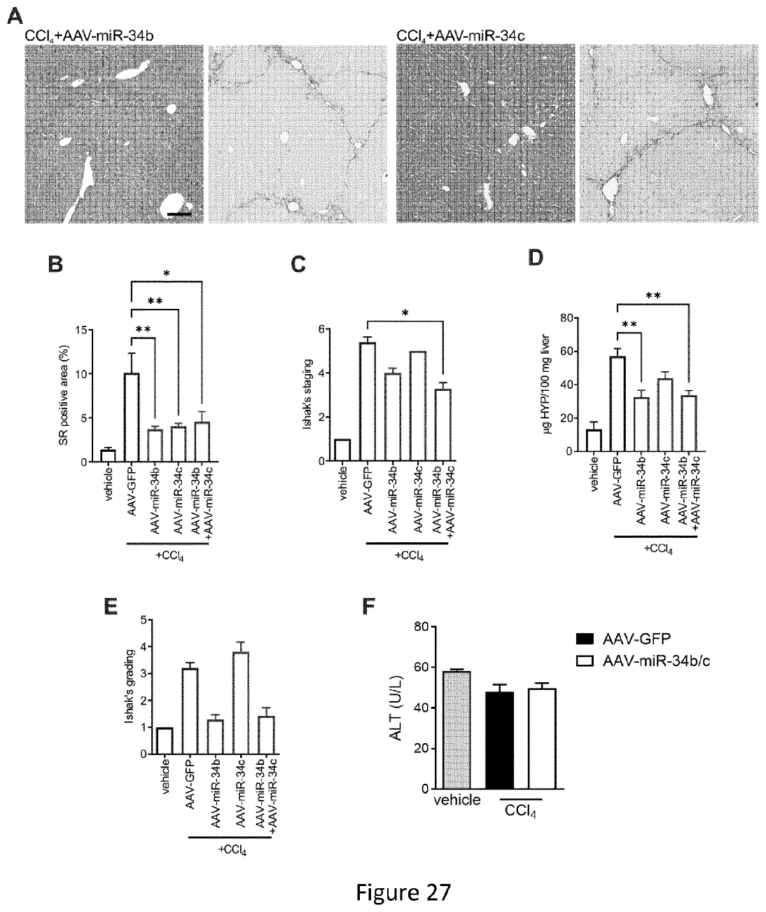

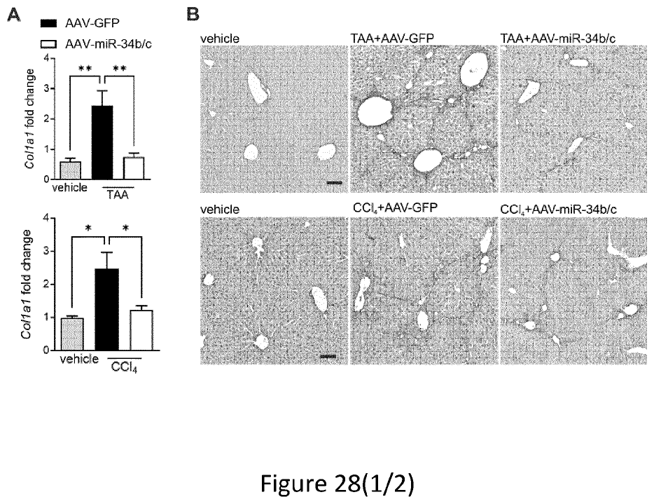

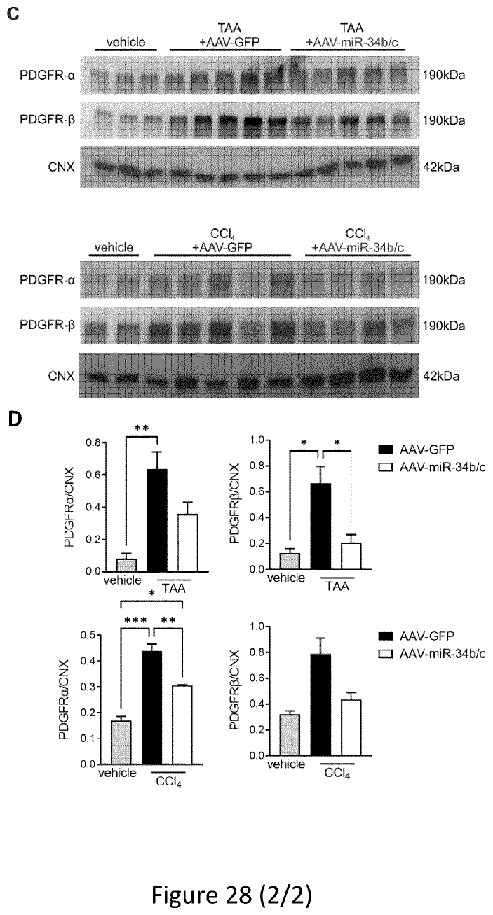

To confirm miR-34b/c anti-fibrotic effect, wild-type mice were also treated with CCl₄ or vehicle for 12 weeks ¹⁷ and at 10 weeks of treatment, they were injected intravenously with AAV-miR-34b, AAV-miR-34c, AAV-miR-34b/c or AAV-GFP, and sacrificed 4 weeks later (FIG. 6A). Consistent with results observed in TAA-treated animals, CCl₄ induced advanced fibrosis or cirrhosis (FIGS. 6B-D) in AAV-GFP injected mice while livers from mice injected with AAV-miR-34b/c and miR-34b/c overexpression showed a lower degree of fibrosis (FIGS. 6B-D), confirmed by a significant reduction in hepatic hydroxyproline content (FIG. 6E), and expression of fibrosis marker genes (FIG. 6F) compared to AAV-GFP treated control mice. Administration of AAV-miR-34b alone and, to a lesser extent AAV-miR-34c, also resulted in amelioration of liver fibrosis (FIGS. 27A-D). In contrast to TAA-induced fibrosis, necro-inflammatory activity is significantly reduced by miR-34b/c overexpression (FIG. 7A). However, inflammatory gene expression is unchanged between vehicle- and CCl₄-treated animals (FIG. 7B). No increase in circulating ALT was found in CCl₄-versus vehicle-treated animals (FIG. 7C). Reduced COL1A1 expression and deposition, and PDGFR-a/B were detected in both TAA- and CCl₄-treated mice that were injected with AAV-miR-34b/c (FIG. 28). Taken together, these findings demonstrate that hepatocyte overexpression of miR-34b/c exerts anti-fibrotic activity in mice with advanced liver fibrosis by downregulating PDGF pathway and collagen biosynthesis. Moreover, data support a stronger anti-fibrotic effect by miR-34b compared to miR-34c.

### Discussion

Liver fibrosis and cirrhosis are major health problems worldwide with an estimated prevalence of up to 25% and 2% in the general population, respectively ³⁶. At least in its initial stages, liver fibrosis can be reversed if the underlying insult is removed. However, this is not possible for several chronic liver diseases and once cirrhosis is established, treatments are limited to management of complications whereas organ transplantation remains restricted to few selected patients. Our understanding of the complex pathomechanisms resulting in liver fibrosis has greatly improved in the last two decades and several clinical interventional studies stemmed from this knowledge ³⁷. Nevertheless, obeticholic acid that is indicated for primary biliary cholangitis remains the only anti-fibrotic approved drug ^{38,39}. Clearly, there is an urgent need for novel and effective antifibrotic drugs.

In this study inventors have shown an anti-fibrotic activity of miR-34b/c. The miR-34 family is composed of three members, miR-34a, -34b, and -34c. MiR-34b and miR-34c are linked as a bi-cistronic transcriptional unit³². The miR-34 family was found to be increased in animal models of liver fibrosis ⁴⁰⁻⁴² and in human patients with different stages of liver fibrosis ⁴³. l While evidence supports a pro-fibrotic role for miR-34a ⁴⁴⁻⁴⁷, the involvement of miR-34b/c in liver fibrosis has been less clear. Similarly to miR-34a, miR-34c targets peroxisome proliferator-activated receptor γ (PPARγ), an anti-fibrotic transcription factor inhibiting HSC activation ⁴⁸ and, based on this finding a pro-fibrotic activity has been hypothesized also for miR-34c ⁴⁴. In contrast, miR-34c-3p has been found to inhibit HSC activation ⁴⁹. However, it should be noted that these studies were both performed in vitro without co-culturing with other liver cell lines whereas in this study, both co-culturing experiments and in vivo studies provide stronger evidence for an anti-fibrotic activity of miR-34b/c. Moreover, we found miR-34b/c directly targets the gene encoding type I collagen and other genes involved in collagen biosynthesis and deposition.

Mir-34b/c role in protecting liver from damage appeared to be specific to fibrosis rather than secondary to a cell protective effect. Analysis of ALT levels in TAA-treated animals showed no significance differences in miR-34b/c^{-/-} mice and miR-34b/c overexpressing mice compared to controls, suggesting that miR-34b/c is not hepatoprotective. Effect of miR-34b/c on inflammation is more difficult to dissect, because while miR-34b/c deletion resulted in upregulation of inflammatory genes after fibrosis induction, miR-34b/c overexpression resulted in reduced necroinflammation in CCl₄— but not in TAA-treated animals. Comprehensive analysis of miR-34b/c targets in different liver cell types and particularly in Kuppfer cell a may help to clarify if additional components of liver damage may be effectively regulated by miR-34b/c.

Hepatocyte delivery and expression of miR-34b/c resulted significant amelioration of liver fibrosis. Noteworthy, miR-34b/c showed efficacy even in livers with advanced fibrosis or cirrhosis (Ishak 5-6).

Mir-34b/c is highly conserved across species. Mouse and human mature miR-34b only differ for one nucleotide that is not included in the seed sequence, while miR-34c is identical between mouse and humans. Therefore, it is predicted that the effect of miR-3c observed in mice will also be recapitulated in humans.

As a proof-of-principle study, inventors used AAV vector for the delivery of the miR-34b/c. However, in future studies, delivery of non-viral miR-34b/c mimics by repeated administrations might be investigated for miRNA delivery, consistent with several applications of miRNA therapeutics that are increasingly moving into clinical trials ^{50,51}. Miravirsen, an anti-miR against miR-122, and RG-012, an antimiR targeting miR-21, are currently in phase II trials for HCV infection (NCT02031133) and Alport syndrome (NCT02855268), respectively. Moreover, a miR-34a mimic is under study for various types of solid tumors, including HCC (NCT01829971). However, this trial has been halted in phase I due to severe, immune-related adverse effects ⁵². While this immunogenicity issue needs to be carefully evaluated, it should be noted that it occurred in patients with advanced tumor who have severe dysregulation of the immune system and has not been observed in other trials. Delivery of miR-34b/c might also be performed by non-viral carriers that are increasingly used in clinical trials ^{51,53}.

In conclusion, it is herein shown the therapeutic potential of miR-34b/c as antifibrotic treatment.

### EXAMPLE 2

### Mouse Studies

Mouse procedures were approved by the Italian Ministry of Health. Male 4- to 15-week-old C57BL/6 (Charles River Laboratories), PiZ ⁵⁴, PiZ/Jnkl^{-/-}, miR-34b/c^{-/-≠}, PiZ/miR-34^{-/-}, Abcb4^{-/-} 55 mice were used. Synthetic miR-914 has been described elsewhere ⁵⁶. rAAV8pCB-mir914-GFP and rAAV8pCB-GFP were generated, purified, and titered by the UMass Gene Therapy Vector Core as previously described ⁵⁷. Bile duct ligation was performed in C57BL/6 mice as previously described ⁵⁸ and mice were sacrificed one week post-surgery. TAA (Sigma-Aldrich) was dissolved in phosphate buffered saline (PBS) and administered to C57BL/6 mice by intraperitoneal injection three times a week for four weeks with escalating doses, starting from 50 mg/kg to 200 mg/kg, as previously described ¹⁷. CCl₄ (Sigma-Aldrich) was dissolved in corn oil (Sigma-Aldrich) administered to C57BL/6 mice by gavage three times a week for four weeks with escalating doses, starting from 0.875 ml/kg to 2.5 ml/kg, as previously described ¹⁷. At sacrifice, mice were perfused with PBS. ALT levels were measured by scil VitroVet analyzer (Scil vet).

### Human Liver and Serum Samples

Human liver samples were collected snap-frozen and anonymized according to human studies approvals obtained from the Institute of Pathology, University Hospital of Basel, Switzerland and St. Louis University School of Medicine, Cardinal Glennon Children's Medical Center. Human liver samples were collected snap-frozen and anonymized according to human studies approvals. Liver specimens from patients with milder liver disease and AAT deficiency confirmed on pathology, and the corresponding controls were anonymously obtained from the Institute of Pathology, University Hospital of Basel, Switzerland (Table 3). Liver samples with severe disease were anonymously obtained from St. Louis University School of Medicine, Cardinal Glennon Children's Medical Center from patients under 18 years of age homozygous for the Z allele of SERPINA1 who underwent hepatic transplantation for liver failure. Control liver specimens were obtained from age-matched patients homozygous for the wild-type allele of the AAT who also underwent liver transplantation. Pathology features of these specimens were previously reported ⁵⁹.

### RNA-seq and GSEA

Libraries preparation was performed with a total of 100 ng of RNA from each sample using QuantSeq 3′mRNA-Seq Library prep kit (Lexogen) according to manufacturer's instructions. Amplified fragmented cDNA of 300 bp in size were sequenced in single-end mode by NextSeq500 (Illumina) with a read length of 75 bp. Sequence reads were trimmed using Trim Galore software ⁶⁰ to remove adapter sequences and low-quality end bases (Q <20). Alignment was performed with STAR ²¹ on the reference provided by UCSC Genome Browser ⁶¹. Gene expression levels were determined with htseq-count ²³ using the Gencode/Ensembl gene model. Differential expression analysis was performed using edgeR ⁶² . Putative miR-34b/c target genes were identified by DIANA-microT-CDS software, with threshold set to 0.7 ^{28,29}. GSEA was performed using the GSEA software (www.broadinstitute.org/gsea) ²⁵. Mouse FOXO3 target gene set was previously reported 63. Liver fibrosis gene set was generated combining previously identified expression signatures in different models of hepatic fibrosis ^{64,65}. Data were deposited in GEO with the accession number GSE141593.

miRNA Analyses

Small RNA libraries were constructed using a TruSeq small RNA sample preparation kit (Illumina) following the manufacturer's protocol. Small RNA-seq libraries were generated using lug of total RNA as input from each sample. By multiplexing, up to 12 samples were combined into a single lane to obtain sufficient coverage and two technical replicates were run for each library. Cluster generation was performed on Flow Cell v.3 (TruSeq SR Cluster Kit v.3; Illumina) using cBOT. Sequencing was performed on the HiSeq1000 platform. Each library was loaded at a concentration of 8-10 pM. Reads were trimmed to remove adapter sequences and low-quality ends and the resulting sequences shorter than 16 nucleotides were discarded. Reads mapping to contaminating sequences (e.g., ribosomal RNA, phIX control) were filtered-out. Filtered reads were aligned both to the human genome (hg19) and to human mature and precursor (hairpin) miRNAs (miRBase v.20) ⁶⁶ using CASAVA software (Illumina). Reads were aligned and grouped on the sequence of the mature miRNAs allowing up to two mismatches within the exact length of the reference mature sequence (i.e., excluding trimming or extension variants). Differential expression analysis of read counts was performed using the Generalized Linear Model approach implemented in the Bioconductor package edgeR ^{62.67}. To remove noise from lowly expressed miRNAs, we performed a Kolmogorov-Smirnov test to measure the distance between samples that were re-sequenced on different lanes. Minimal distance was found at a cut-off of 9 reads and thus, all miRNAs with a read count greater than 9 in at least two samples were retained. Heatmap and volcano plot were generated in R environment ⁶⁸. Data were deposited in GEO with the accession number GSE85413. For targeted miRNA expression analysis in mice and in human livers with mild liver disease, miRNA-enriched total RNA was extracted from liver tissues using miRNeasy Mini Kit and from plasma using miRNeasy serum/plasma kit (QIAGEN). 10-20 ng of total RNA were reverse transcribed using TaqMan MicroRNA Reverse Transcription Kit and TaqMan miRNA assay (Table 4) (Applied Biosystems). The qPCR was performed in triplicate using 1-3 μl of cDNA, TaqMan MicroRNA assay and TaqMan Universal Master Mix II no UNG (Applied Biosystems) on a Roche Light Cycler 480 system (Roche). Running program was as follows: preheating, 10 minutes at 95° C.; 40 cycles of 15seconds at 95° C. and 60 seconds at 60° C. SnoRNA234, miR-23a and miR-152 were used as housekeeping for mouse livers and plasma, and human livers, respectively. For pri-miR-34b/c analysis, isolation of hepatocytes and non-parenchymal liver cells from PiZ mouse livers was performed as previously described ⁵⁹. 2 μg of total RNA were retrotranscribed using High Capacity cDNA Reverse Transcription kit according to manufacturer's protocol (Applied Biosystems). The qPCR was performed using TaqMan pri-miRNA assay (Table 4) as for mature miRNAs. 18S was used as housekeeping. Data analysis was performed using LightCycler 480 software version 1.5 (Roche). To validate Pdgfra and Pdgfrb as targets of miR-34b/c, murine Pdgfra and Pdgfrb 3′untranslated regions (UTR) were amplified by PCR from genomic DNA of C57BL/6 wild-type mice and cloned downstream the firefly luciferase gene into pmirGLO Dual-Luciferase miRNA Target Expression Vector (Promega). The miR-34b/c 8-mer recognition sites in the pmirGLO Pdgfra.3′UTR and pmirGLO Pdgfrb.3′UTR plasmids were mutagenized by QuickChange site-directed mutagenesis kit (Agilent) according to manufacturer's instructions. Primers used for construct generation are shown in Table 5. HeLa cells were cultured in DMEM plus 10% fetal bovine serum (FBS) and 5% penicillin/streptomycin. Cells were co-transfected with the plasmid containing the wild-type or mutagenized pmirGLO Pdgfra.3′UTR and pmirGLO Pdgfrb.3′UTR and with negative control, miRIDIAN mimic miR-34b-5p, or miRIDIAN Mimic miR-34c-5p using Interferin transfection reagent (Polyplus). Cells were harvested 48 hours after transfection and assayed for luciferase activity using the Dual-Luciferase Reporter (DLR™) Assay System (Promega). Data were expressed relative to renilla luciferase activity to normalize for transfection efficiency. Two sets of experiments were performed with n=3 in each experiment. Firefly-to-renilla activity ratio for each replicate was normalized to the average of negative control transfected samples.

For analyses of miRNA in human livers with end stage liver disease, total RNA was isolated from liver tissue with Trizol (Life Technologies) and diluted to 10 ng/μl. miR-34c and U47 small RNAs were jointly retro-transcribed using small RNA-specific stem-loop primers (Life

Technologies) and Taqman miRNA Reverse Transcription Kit (Life Technologies). Droplets were generated using a QX-200 Droplet Generator (Biorad) and endpoint droplet digital PCR (ddPCR) was performed using small RNA-specific primers and FAM-labeled probes (Life Technologies), and 2× ddPCR Supermix for Probes no dUTP (Biorad). Positive and negative droplets were quantified (Biorad). Only samples with at least 10,000 accepted droplets and at least 100 negative droplets were included in the data analysis. Samples were run in triplicates, the replicates averaged, and the ratio of miR-34c-positive/U47-positive droplets was calculated.

Liver staining, Laser Controlled Microdissection and Western blotting

Livers from PBS-perfused mice were fixed in 4% paraformaldehyde for 12 hours, stored in 70% ethanol, and embedded into paraffin blocks. PAS-D staining was performed on 5-μm thick paraffin sections of livers. Sections were rehydrated and treated with 0.5% a-amylase type VI-B (Sigma-Aldrich) for 20minutes and stained with PAS reagent according to manufacturer's instructions (Bio-Optica).

Sirius Red staining was performed on 5-μm liver sections which were rehydrated and stained for 1hour in picrosirius red solution (0.1% Sirius red in saturated aqueous solution of picric acid). After two changes of acidified water (0.5% acetic acid in water), sections were dehydrated, cleared in xylene, and mounted in a resinous medium. Images were captured by Axio Scan.Z1 microscope (Zeiss) and analyzed by ImageJ for quantification of Sirius Red positive area. Five images for each mouse were analyzed. For immunohistochemistry, 5-μm thick sections were rehydrated and permeabilized in PBS/0.2-0.5% Triton (Sigma) for 20minutes. Antigen unmasking was performed in 0.01 M citrate buffer in a microwave oven. Next, sections underwent blocking of endogenous peroxidase activity in methanol/1.5% H202 (Sigma) for 30minutes and incubation with blocking solution [(3% BSA (Sigma), 5% donkey serum (Millipore), 1.5% horse serum (Vector Laboratories) 20 mM MgC12, 0.3% Triton (Sigma) in PBS] for 1hour. Sections were incubated with primary antibody (Table S5) over-night at 4° C. and with universal biotinylated horse anti-mouse/rabbit IgG secondary antibody (Vector Laboratories) for 1hour. Biotin/avidin-HRP signal amplification was achieved using ABC Elite Kit (Vector Laboratories) according to manufacturer's instructions. 3,3′-diaminobenzidine (DAB, Vector Laboratories) was used as peroxidase substrate. Mayer's hematoxylin (Bio-Optica) was used as counter-staining. Sections were de-hydrated and mounted in Vectashield (Vector Laboratories). Image capture was performed using Leica DM5000 microscope.

For laser-controlled microdissection, 10-μm sections from formalin-fixed paraffin-embedded PiZ livers were quickly rehydrated (xylene 2 minutes-2 times, 100% ethanol 1minute, 95% ethanol 1minute, 75% ethanol 1minute), stained with Mayer's hematoxylin (Bio-Optica) and eosin Y (Sigma) and quickly dehydrated (75% ethanol 1 minute, 95% ethanol 1minute, 100% ethanol 1minute). Solutions were all prepared in diethyl pyrocarbonate-treated water and kept at 4ºC to minimize RNA degradation. Dried sections underwent laser-controlled microdissection using PALM Microbeam (Zeiss). A total area of 5×10⁵ μm² for each sample was dissected. Serial PAS-D stained sections were used to identify areas for dissection. Total RNA was extracted using RNeasy FFPE kit (Qiagen) on manufacturer's protocol.

For Western blotting, proteins from tissues were extracted in RIPA buffer according to standard procedures. Nuclear protein extracts were prepared using CelLytic NuCLEAR Extraction Kit (Sigma-Aldrich). Primary antibodies were diluted in TBS-T/5% milk (Bio-Rad Laboratories) (Table 6). Secondary antibodies were ECL anti-rabbit HRP and ECL anti-mouse HRP (GE Healthcare). Peroxidase substrate was provided by ECL Western Blotting Substrate kit (Pierce). Analysis of band intensities was performed using Quantity One 1-D Analysis Software version 4.6.7 (Bio-Rad Laboratories).

### Statistical Analyses

Two tailed Student's/test and ANOVA plus Tukey's HSD post-hoc were used as statistical tests for mean comparisons. Experimental group sizes are reported in figure legends. Data are reported as average +standard error.

### RESULTS

Profiling of miRNA in PiZ Mouse Livers Revealed Upregulation of miR-34b/c

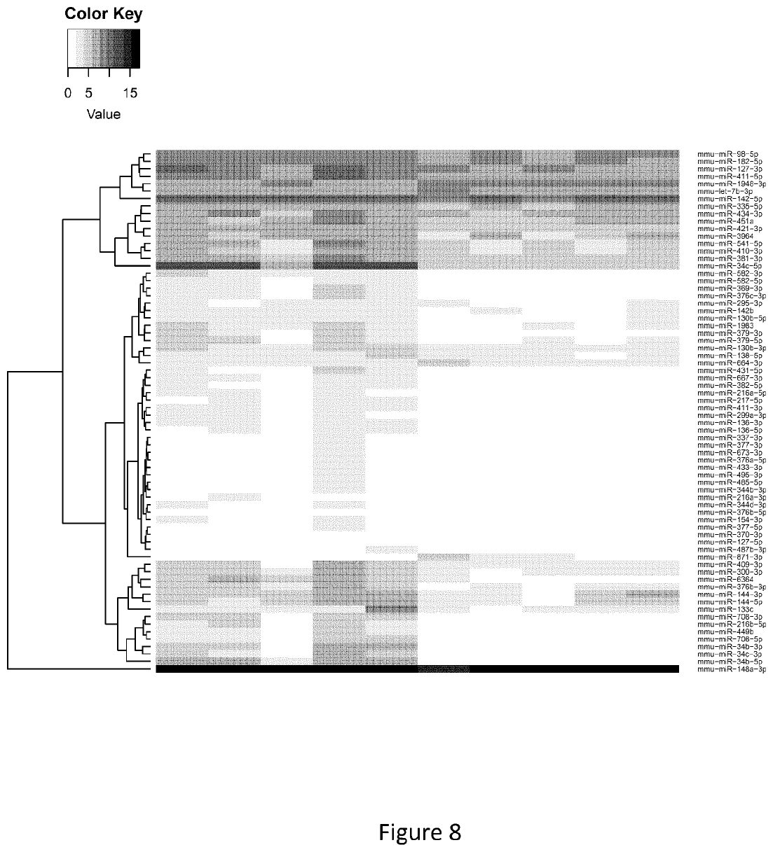

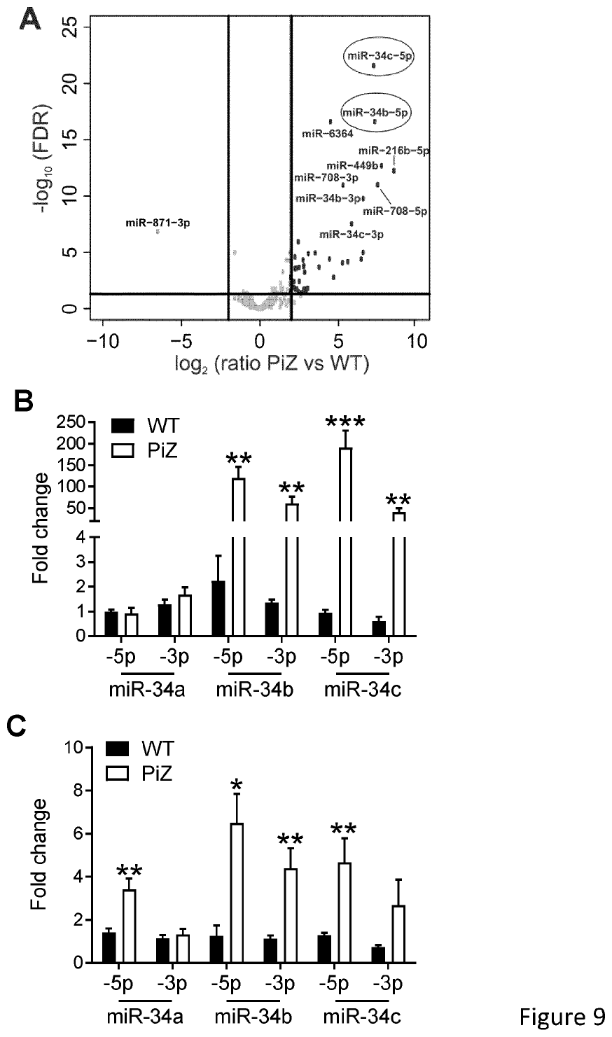

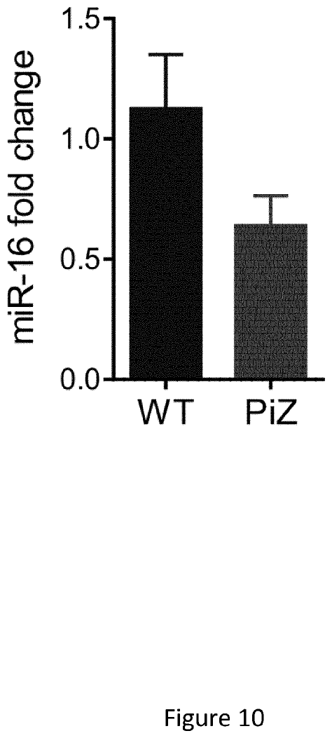

Transgenic PiZ mice accumulate ATZ within the ER of hepatocytes in a manner akin to that of patients affected by AAT deficiency. Therefore, these mice are a valuable experimental model for investigating the liver disease of AAT deficiency ⁵⁴. Inventors evaluated differentially expressed miRNA by next generation sequencing of miRNA in the livers of PiZ mice and strain-, age-, and sex-matched wild-type controls. Seventy miRNAs were found to be differentially expressed in PiZ livers compared to wild-type controls (FIG. 8). Among differentially expressed miRNAs, both miR-34b-5p and miR-34c-5p (henceforth referred to as miR-34b and miR-34c) showed the highest fold changes and statistical significance in PiZ mice compared to wild-type controls (FIG. 9A). In PiZ mice, upregulation of miR-34b and miR-34c, which are expressed from a common primary transcript32, was confirmed in both liver (FIG. 9B) and plasma (FIG. 9C) by targeted real time PCR analysis whereas differences in miR-34a expression were detected in blood but not in liver (FIGS. 9B-C). Expression of miR-16 a marker of hemolysis69, was not significantly different between the two groups (FIG. 10), ruling out hemolysis as the factor responsible for the increased miR-34b/c levels in the blood.

miR-34b/c is Mainly Expressed by Hepatocytes and its Levels Correlated with Hepatic ATZ Accumulation

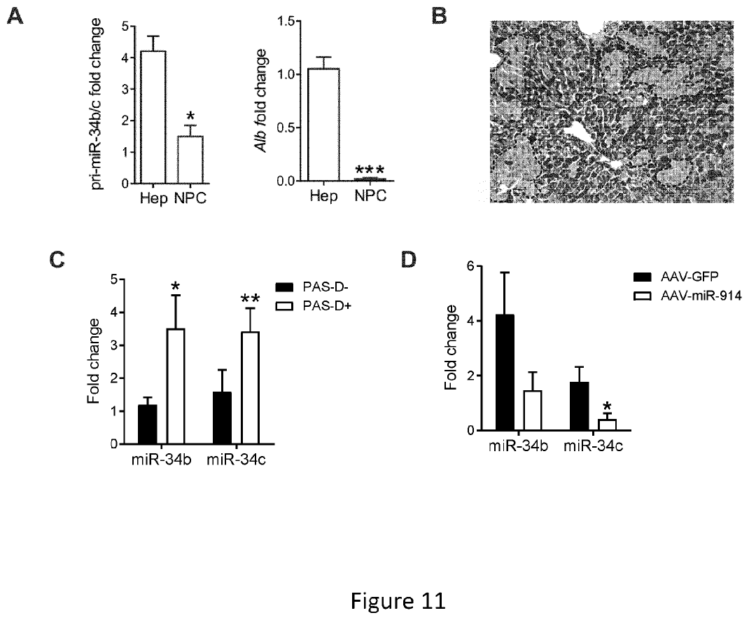

To investigate the role of miR-34b/c in ATZ-mediated liver disease, first inventors measured miR-34b/c expression in parenchymal and non-parenchymal liver cells. Because mature miRNAs can be secreted and taken up by neighboring cells not expressing the miRNAs, inventors evaluated the miR-34b/c common primary transcript (pri-miR-34b/c) in parenchymal and non-parenchymal liver cells of PiZ mice. This precursor transcript is specific for the cells expressing the miRNAs but not for cells that take up the mature miRNAs. The pri-miR-34b/c was found to be mainly expressed in the parenchymal fraction enriched for albumin gene expression (FIG. 11A). ATZ accumulation visualized by periodic acid-Schiff staining after diastase digestion (PAS-D) in PiZ mouse liver is not uniform and typically regions both devoid and containing PAS-D globules are detected on the same liver tissue section ⁷⁰. To correlate ATZ accumulation with miR-34b/c levels, inventors performed laser-controlled microdissection (LCM) on PiZ livers to separate PAS-D-negative from PAS-D-positive regions for qPCR analysis of miR-34b/c expression. Compared to PAS-D-negative regions, PAS-D-positive regions showed increased miR-34b/c (FIG. 11B-C). Next, inventors analyzed 6-week-old PiZ mice injected with a recombinant serotype 8 adeno-associated virus (rAAV8) vector that expresses an artificial miRNA designed to target and downregulate expression of ATZ (AAV8pCB-mir914-GFP) or injected with the same dose of an AAV8pCB-GFP expressing the green fluorescent protein (GFP) reporter gene ⁵⁶. By four weeks post-injection, livers of mice injected with AAV8pCB-mir914-GFP showed a reduction of PAS-D staining and circulating levels of ATZ ⁵⁹, and decreased miR-34b/c liver content (FIG. 11D). Taken together, these findings revealed a correlation between miR-34b/c levels and ATZ accumulation in the liver.

### MiR-34b/c Expression is Upregulated by JNK-FOXO3

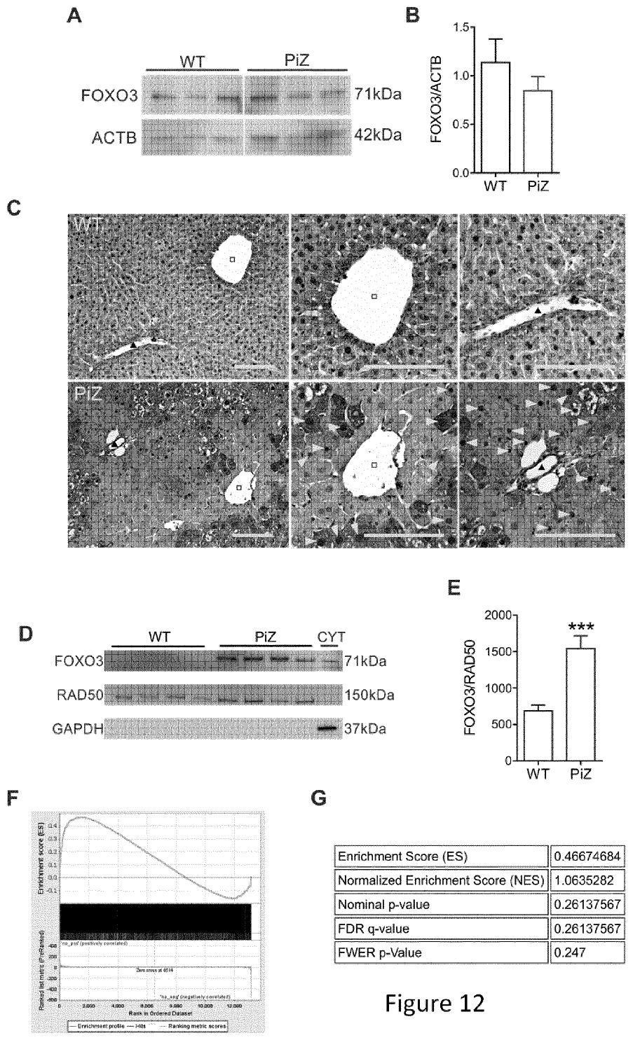

Expression of miR-34b/c is directly regulated by FOXO3 ^{71.72}. However, FOXO3 protein levels showed only mild and non-significant differences between PiZ and wild-type control mice (FIGS. 12A-B). Nevertheless, PiZ mice showed increased FOXO3 nuclear signals in hepatocytes by immunohistochemistry (FIG. 12C) that was confirmed by detection of higher FOXO3 levels in the nuclear fractions of PiZ mouse livers compared to wild-type controls, suggesting increased nuclear translocation (FIGS. 12D-E). Consistent with increased nuclear FOXO3, Gene Set Enrichment Analysis (GSEA) on RNA-seq data revealed a significant enrichment of FOXO3 target genes among differentially expressed genes in PiZ livers compared to wild-type controls (Enrichment Score [ES]=0.47) (FIGS. 12F-G).

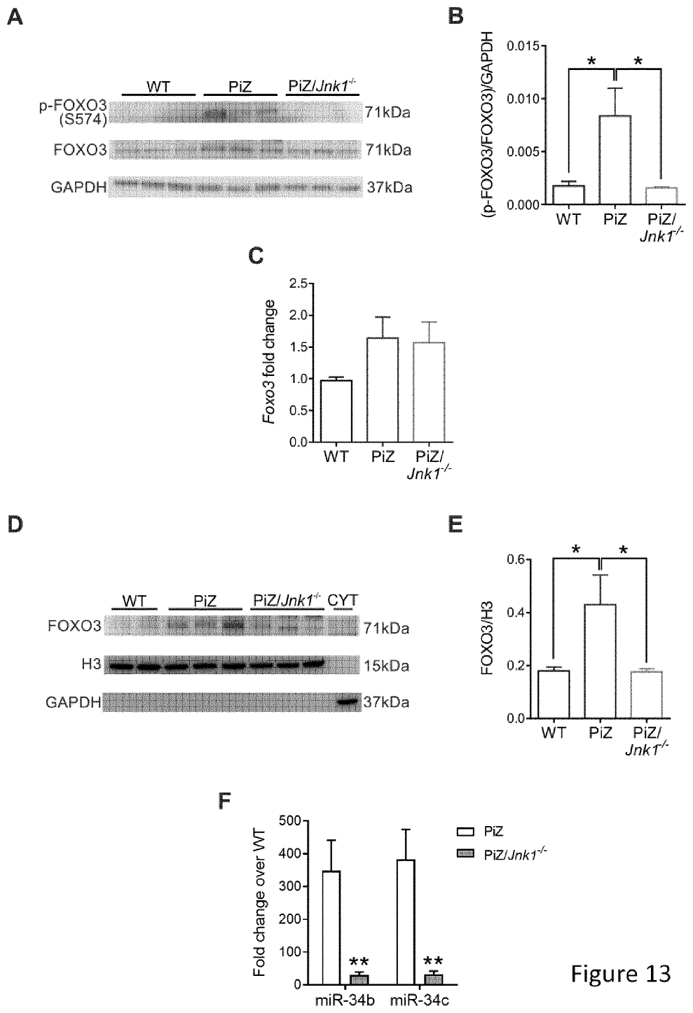

FOXO3 is activated by JNK phosphorylation on residue Ser^{574 73} and JNK is activated in PiZ livers74. PiZ mice showed increased levels of phospho-Ser⁵⁷⁴-FOXO3 whereas PiZ/Jnk1^{-/-} mice showed levels of phosphorylated FOXO3 similar to wild-type controls (FIGS. 13A-B), despite no significant changes in Foxo3 gene expression (FIG. 13C), and reduced nuclear FOXO3 compared to PiZ (FIGS. 13D-E). Moreover, PiZ/Jnk1^{-/-} livers showed reduced levels of miR-34b/c compared to PiZ control (FIG. 13F). Taken together, these results suggest that JNK-dependent activation of FOXO3 drives miR-34b/c expression in PiZ livers.

Lack of miR-34b/c Accelerates the Development of Liver Fibrosis in PiZ Mice

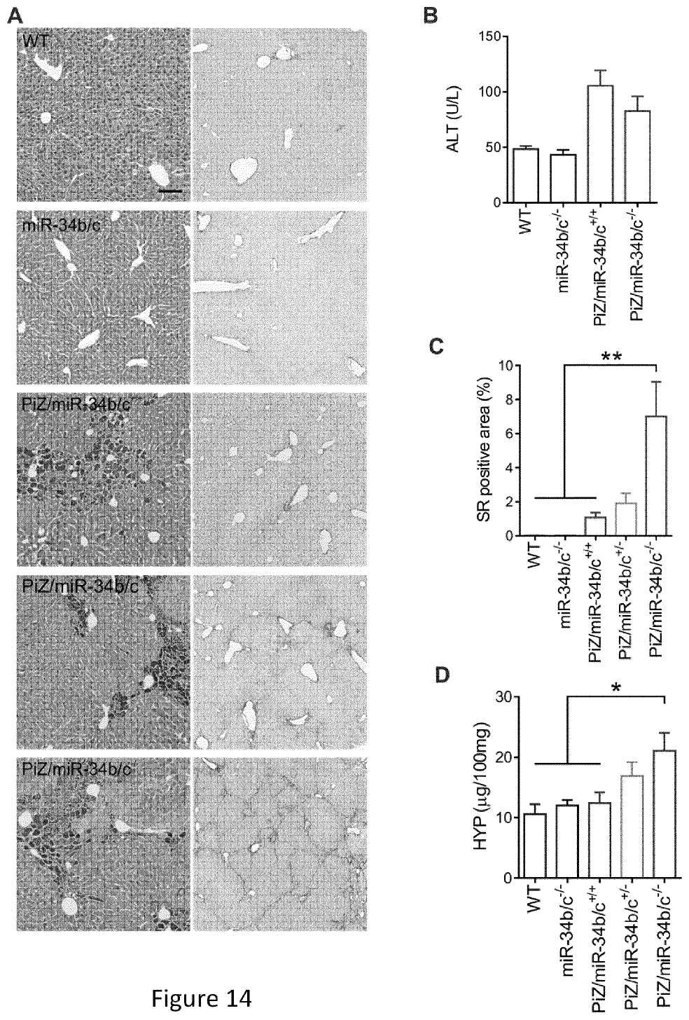

To investigate the role of miR-34b/c in ATZ-mediated liver disease, inventors generated PiZ/miR-34b/c^{-/-} by crossing PiZ mice with miR-34b/c^{-/-} mice 16. PiZ/miR-34b/c^{-/-} mice showed normal fertility and sex ratio. At 13-15 weeks of age, PiZ/miR-34b/c^{+/+},PiZ/miR-34b/c^{+/-} and PiZ/miR-34b/c^{-/-} livers showed similar ATZ accumulation by PAS-D staining (FIG. 14A, left panels). Circulating alanine aminotransferase (ALT) levels were only mildly increased in PiZ/miR-34b/c^{+/+} and PiZ/miR-34b/c^{-/-} compared to controls and not significantly different between PiZ/miR-34b/c^{-/-} and PiZ/miR-34b/c^{+/+} mice (FIG. 14B). Nevertheless, livers of PiZ/miR-34b/c^{-/-} and PiZ/miR-34b/c^{+/-} mice developed more severe fibrosis than PiZ/miR-34b/c^{+/+}, as shown by Sirius Red staining (FIG. 14A, right panels). Quantification of Sirius Red staining and measurement of hepatic hydroxyproline content confirmed a significant increase in liver fibrosis in PiZ/miR-34b/c^{-/-} compared to PiZ/miR-34b/c^{+/+},whereas PiZ/miR-34b/c^{+/-} showed an intermediate level of fibrosis between those of PiZ/miR-34b/c^{-/-} and PiZ/miR-34b/c^{+/+} (FIGA. 14C-D).

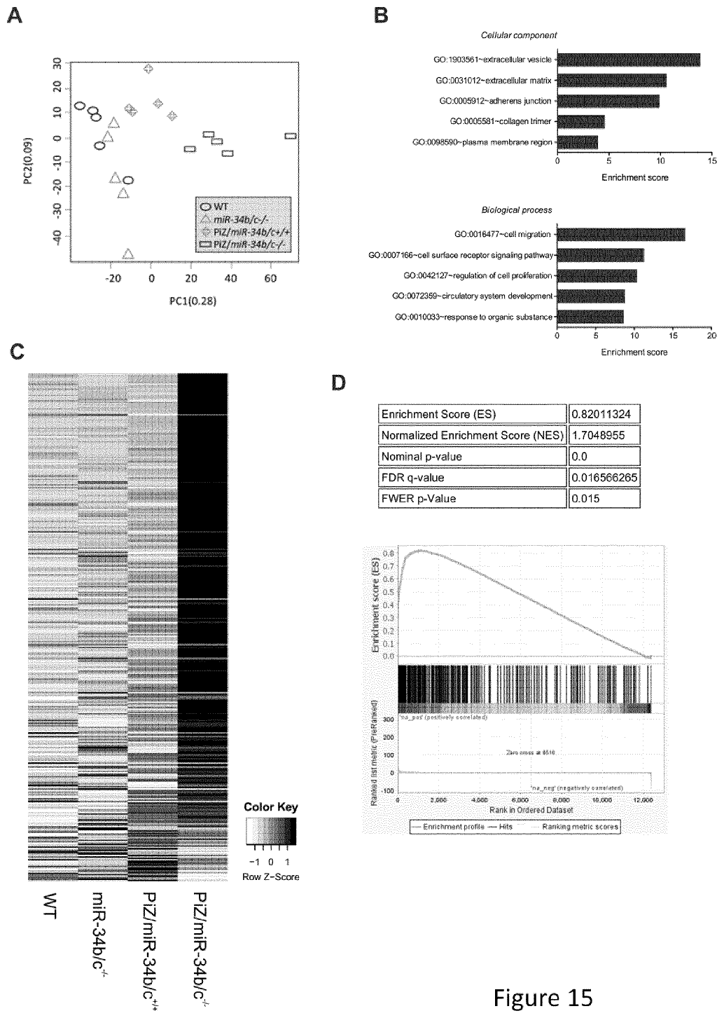

RNA-seq analysis showed that hepatic gene expression in PiZ/miR-34b/c^{-/-} mice was well clustered and separated from PiZ/miR-34b/c^{+/+}, miR-34b/c^{-/-}, and wild-type control mice (FIG. 15A). Analysis of differentially expressed genes in PiZ/miR-34b/c^{-/-} versus PiZ/miR-34b/c^{+/+} yielded 1,580 dysregulated genes (802 up- and 778 down-regulated). Functional annotation clustering analysis showed that a significant amount of the differentially expressed genes is associated with biological processes of extracellular matrix components, including collagens, tissue damage, and regeneration (i.e., cell death and proliferation, angiogenesis, cell migration) (FIG. 15B). Moreover, GSEA using a liver fibrosis expression signature ^{64,65} showed a significant enrichments of fibrosis genes among the upregulated genes in PiZ/miR-34b/c^{-/-} versus PiZ/miR-34b/c^{+/+} (FIGS. 15C-D). Taken together these findings support a protective role of miR-34b/c in the development of liver fibrosis in PiZ mice.

Activation of Platelet-Derived Growth Factor (PDGF) Pathway in PiZ Mice Lacking miR-34b/c

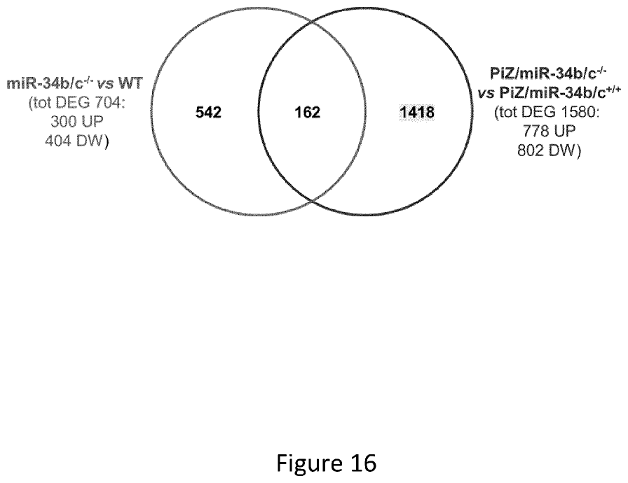

To investigate the anti-fibrotic mechanism of miR-34b/c in livers expressing ATZ, inventors searched for genes upregulated in miR-34b/c^{-/-} livers with fibrosis. By Venn diagram, inventors compared genes differentially expressed in miR-34b/c^{-/-} versus wild-type and in PiZ/miR-34b/c^{-/-} versus PiZ/miR-34b/c^{+/+} livers. Next, inventors isolated genes that were differentially expressed in PiZ/miR-34b/c^{-/-} versus PiZ/miR-34b/c^{+/+} but not in miR-34b/c^{-/-} versus wild-type livers (FIG. 16). Within this subset of 1418 genes, 58 upregulated genes were miR-34b/c putative target genes (Table 7). Among these genes, Pdgfra and Pdgfrb, encoding the α and β subunits of the platelet-derived growth factor receptor (PDGFR) respectively, were the most interesting because of the consolidated role of the platelet-derived growth factor (PDGF) pathway in liver fibrosis. PDGFA-D ligands are potent mitogens that drive hepatic stellate cell proliferation and differentiation into myofibroblasts through activation of tyrosine kinase PDGFR75

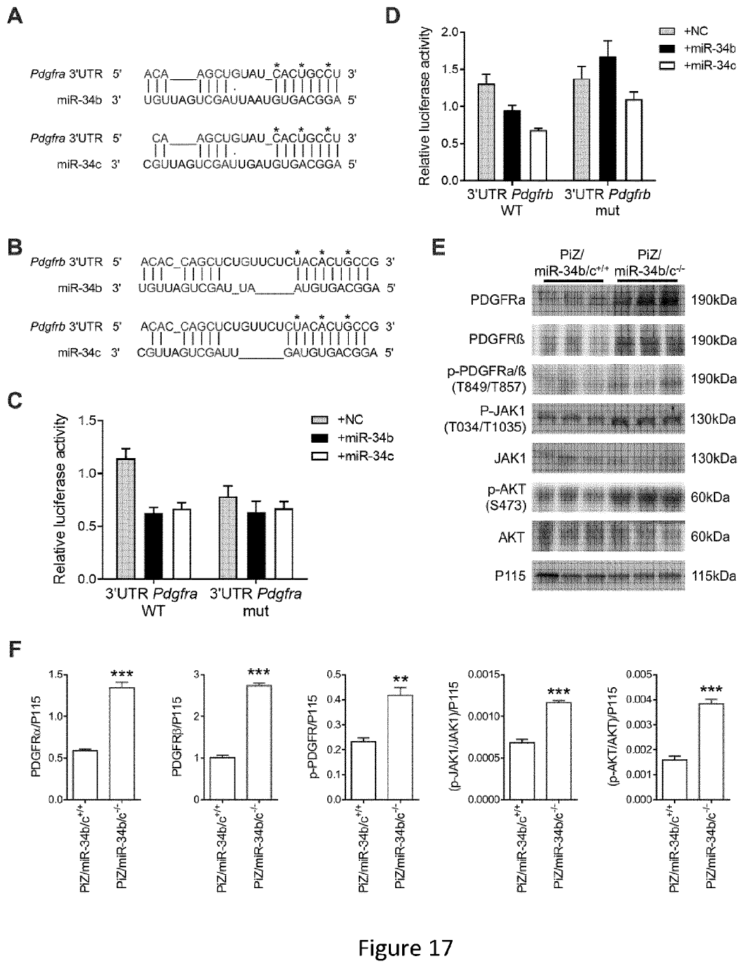

Downregulation of PDGFRa or PDGFRβ encoded by Pdgfra and Pdgfrb respectively, exerts a protective role against liver fibrosis, while their overexpression is pro-fibrotic ⁷⁶⁻⁷⁸. Interestingly, the Pdgfra 3′-untranslated region (UTR) includes two putative canonical target sites (one 8-mer and one 7-mer) for miR-34b/c. The 3′-UTR of Pdgfrb has six canonical binding sites for miR-34b/c (one 8-mer and five 6-mer) and one 8-mer site in the coding sequence. Moreover, PDGFRA and PDGFRB targeting by the miR-34 family has been validated in humans ³⁰. Targeting by miR-34b/c was validated by luciferase assay on wild-type and mutagenized 3′-UTR from Pdgfra (FIGS. 17A,C) and Pdgfrb (FIGS. 17B,D). Upregulation of Pdgfra and Pdgfrb was confirmed at the protein level in PiZ/miR-34b/c^{-/-} versus PiZ/miR-34b/c^{+/+} (FIGS. 17E,F). Moreover, livers from PiZ/miR-34b/c˜ showed increased levels of phospho-Tyr^{849/857}. PDGFRα/β and increased phosphorylation of its targets JAK1 and AKT, consistent with released miR-34b/c repression of PDGFRα/β and activation of its downstream targets (FIGS. 17E,F). Taken together these data show that lack of miR-34b/c results in upregulation of PDGFRα/β and activation of the PDGF pathway, thus suggesting that miR-34b/c antagonize liver fibrosis through repression of the PDGF pathway.

miR-34c is Increased in Human Livers Expressing ATZ

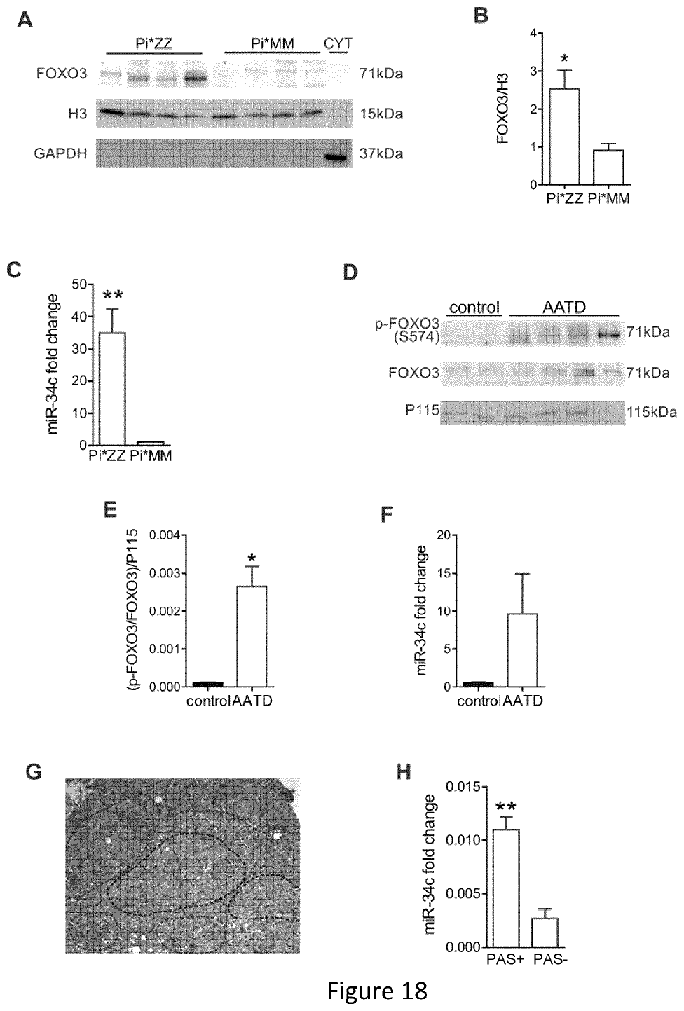

To investigate the clinical relevance of our findings, inventors analyzed FOXO3 and miR-34b/c in liver samples from patients with AAT deficiency. Because miR-34b and miR-34c are both expressed from the same primary transcript in humans³⁹, inventors analyzed human miR-34c levels as a proxy for miR-34b/c expression. Similar to PiZ mice, AAT deficient patients with advanced hepatic disease requiring liver transplantation⁵⁸ showed increased FOXO3 nuclear levels and significant miR-34c upregulation compared to control livers from patients who underwent liver transplantation for unrelated liver disorders (FIGS. 18A-C). Noteworthy, livers of patients with AAT deficiency were previously found to have activation of JNK⁷⁴. Moreover, liver specimens from four independent patients with milder liver disease (Table 3) showed increased phospho-Ser⁵⁷⁴-FOXO3 levels, compared to controls with unrelated liver disease (FIG. 18D-E) and a trend towards upregulation of miR-34c compared to controls (FIG. 18F). Consistent with the data in PiZ livers, miR-34c was upregulated in PAS-D-positive liver areas compared to globule-devoid areas obtained by LCM (FIG. 18G, H).

JNK/FOX03/miR-34b/c pathway is activated in liver fibrosis of different etiologies

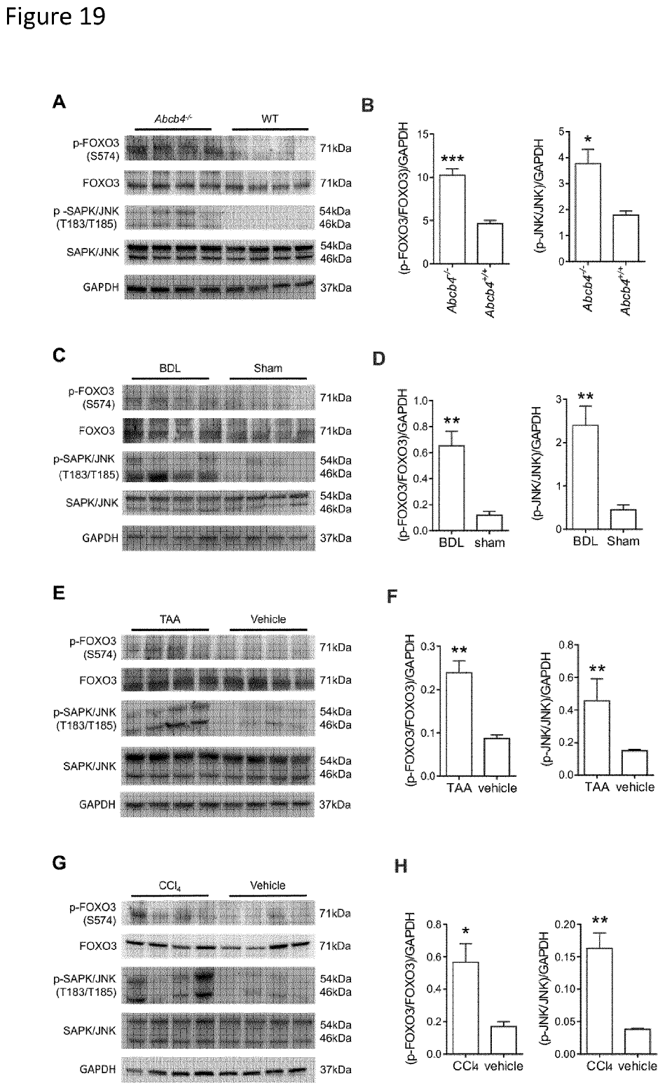

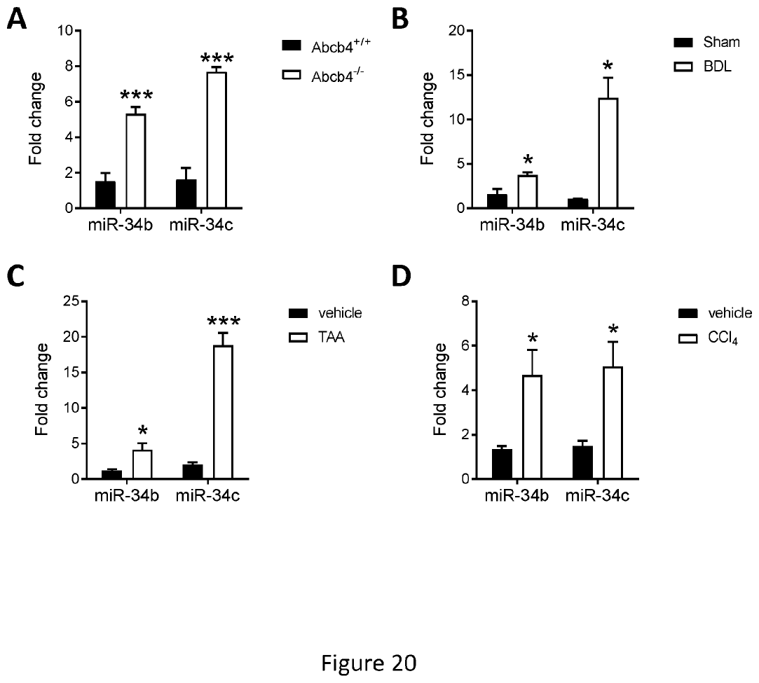

Inventors hypothesized that miR-34b/c and its upstream regulators JNK and FOXO3 may be involved in other forms of liver fibrosis besides AAT deficiency. To investigate this hypothesis, inventors evaluated JNK, FOXO3, and miR-34b/c in various models of liver fibrosis, including biliary fibrosis in Abcb4^{-/-} mice ⁷⁹ and in mice with bile duct ligation 58, and pharmacologically-induced pan-lobular fibrosis in mice administered with carbon-tetrachloride (CCl₄) or thioacetamide (TAA) ¹⁷. Consistent with previous studies ^{80,81}, JNK activation and concomitant increased phospho-Ser⁵⁷⁴-FOXO3 were detected in fibrotic livers compared to controls (FIG. 19). Moreover, miR-34b/c was upregulated in fibrotic livers compared to controls (FIG. 20), confirming activation of the JNK/FOXO3/miR-34b/c pathway in liver fibrosis induced by various etiologies.

### Discussion

In the present study, inventors found JNK phosphorylation on Ser⁵⁷⁴, FOXO3 activation, and miR-34b/c upregulation in murine and human livers with ATZ accumulation. PiZ mice deleted for miR-34b/c showed greater liver fibrosis and increased signaling of PDGF, an established pro-fibrotic molecule that is a target of miR-34b/c. Interestingly, JNK-activated FOXO3 and miR-34b/c upregulation were also found in several mouse models of liver fibrosis, suggesting that this pathway is broadly implicated in hepatic diseases.

JNK signaling is associated with cell death, survival, differentiation, proliferation, and tumorigenesis in hepatocytes. Moreover, it is involved in inflammation and fibrosis ⁸². JNK is activated in livers expressing ATZ ⁷⁴ and can phosphorylate FOXO3, thus promoting its nuclear translocation ⁸³. As previously observed with HCV infection ⁷³, JNK can phosphorylate FOXO3 on Ser⁵⁷⁴ residue in livers expressing ATZ. Ser⁵⁷⁴ phosphorylation drives FOXO3-dependent apoptosis ⁷³ and consistently, a correlation between apoptosis and the amount of ATZ has been previously shown ⁸⁴. Previous studies have reported that FOXO3 binds to the miR-34b/c promoter upregulating its expression ^{71,72} and accordingly, inventors found that upregulation of miR-34b/c in PiZ mice was dependent on FOXO3 activation by JNK. Interestingly, our findings suggest a previously unrecognized anti-fibrotic role for FOXO3-mediated upregulation of miR-34b/c in livers expressing ATZ, as well as in livers of mice subjected to various types of injuries resulting in fibrosis. Upregulation of miR-34 family members was previously found in animal models of pharmacologically induced liver fibrosis and miR-34b upregulation was associated with fibrosis due to viral hepatitis in humans ⁸⁵. While a growing body of evidence supports a pro-fibrotic role for miR-34a⁴⁴⁻⁴⁷, the role of miR-34b/c has been less clear and both anti-fibrotic⁴⁸⁻⁴⁹ and pro-fibrotic activities have been observed⁴⁴. However, most of these studies were performed in vitro without co-culturing of hepatocytes with hepatic stellate cells. In contrast, inventors evaluated the consequences of miR-34b/c deletion in vivo in the PiZ mouse model that spontaneously develops liver fibrosis by 16-24 weeks of age ⁸⁶.

The findings of this study also suggested that miR-34b/c reduces liver fibrosis by repressing PDGF signaling. Activation of the PDGF pathway mainly occurs in hepatic stellate cells and portal fibroblasts, promoting their proliferation and trans-differentiation towards myofibroblasts, which drive the development and progression of fibrosis ⁷⁵. Inventors detected FOXO3 activation and miR-34b/c expression mainly in hepatocytes. Therefore, it could be argued that repression of PDGF signaling by miR-34b/c in hepatocytes protects from liver fibrosis. However, miRNAs can be secreted and the paracrine activity of secreted miR-34b/c on other liver cell types cannot be excluded. Accordingly, miR-34b/c levels were increased in PiZ plasma, supporting their secretion by hepatocytes. On the other hand, PDGFRa expression is induced in damaged hepatocytes and hepatocyte-restricted deletion of PDGFRa decreased liver fibrosis ⁸⁷, supporting an hepatocyte-specific anti-fibrotic effect of miR-34b/c. Nevertheless, the involvement of additional genes unrelated to the PDGF pathway that are still targets of miR-34b/c cannot be excluded.

Interestingly, FOXO3 plays a crucial role in fibrogenesis occurring in idiopathic pulmonary fibrosis, a lethal, progressive fibrosing parenchymal lung disease ⁸⁸. Therefore, the results of the present studies raise the attractive hypothesis that miR34b/c upregulation might also be involved in lung fibrosis.

Liver fibrosis is not very common in young individuals who are homozygotes for the Z allele of SERPINA1, but its incidence increases significantly with age. According to recent studies, clinically relevant fibrosis occurs in 20-35% of adult Pi*ZZ and the degree of fibrosis correlates with the mutant protein burden ^{89,90}. The mechanisms underlying the variability in liver fibrosis development among homozygotes for the Z allele is unknown. Polymorphisms in the promoter region affecting miR-34b/c levels might protect or increase individuals' susceptibility to develop liver fibrosis^{91,92}. The heterozygote Z allele has recently emerged as the strongest single nucleotide polymorphism-based risk factor for cirrhosis in non-alcoholic fatty liver disease (NAFLD) and alcohol misuse ⁹³. Therefore, it can be speculated that miR-34b/c polymorphisms might also increase risk of liver fibrosis also in individuals who are heterozygotes for the Z allele.

In conclusion, inventors identified miR-34b/c upregulation and JNK-FOXO3 activation in livers injured by expression of ATZ or other types of insults resulting in fibrosis. In PiZ mice, lack of miR-34b/c resulted in more severe liver fibrosis, likely as a consequence of increased PDGF signaling. Taken together these results unravel an important pathway implicated in pathogenesis of liver diseases. Fibrosis is a major global health problem and the elucidation of its pathogenic mechanism and hence of key therapeutic targets is a research priority ⁹⁴. Elucidation of molecular mechanisms underlying liver fibrosis is fundamentally important for establishing antifibrotic therapies ⁹⁵ and this study revealed a new pathway that might become a target for therapeutic interventions.

### REFERENCES

1 The global, regional, and national burden of cirrhosis by cause in 195 countries and territories, 1990-2017: a systematic analysis for the Global Burden of Disease Study 2017. The lancet. Gastroenterology & hepatology 5, 245-266, doi:10.1016/s2468-1253(19)30349-8 (2020).
2 Zhang, J. et al. miR-21 Inhibition Reduces Liver Fibrosis and Prevents Tumor Development by Inducing Apoptosis of CD24+ Progenitor Cells. Cancer research 75, 1859-1867, doi:10.1158/0008-5472.CAN-14-1254 (2015).
3 Zhang, Z. et al. The autoregulatory feedback loop of microRNA-21/programmed cell death protein 4/activation protein-1 (MiR-21/PDCD4/AP-1) as a driving force for hepatic fibrosis development. The Journal of biological chemistry 288, 37082-37093, doi:10.1074/jbc.M113.517953 (2013).
4 Caviglia, J. M. et al. MicroRNA-21 and Dicer are dispensable for hepatic stellate cell activation and the development of liver fibrosis. Hepatology 67, 2414-2429, doi:10.1002/hep.29627 (2018).
5 Matsumoto, Y. et al. MiR-29a Assists in Preventing the Activation of Human Stellate Cells and Promotes Recovery From Liver Fibrosis in Mice. Molecular therapy: the journal of the American Society of Gene Therapy 24, 1848-1859, doi:10.1038/mt.2016.127 (2016).
6 Roderburg, C. et al. Micro-RNA profiling reveals a role for miR-29 in human and murine liver fibrosis. Hepatology 53, 209-218, doi:10.1002/hep.23922 (2011).
7 Chen, L., Brenner, D. A. & Kisseleva, T. Combatting Fibrosis: Exosome-Based Therapies in the Regression of Liver Fibrosis. Hepatology communications 3, 180-192, doi:10.1002/hep4.1290 (2019).
8 Chen, L. et al. Epigenetic regulation of connective tissue growth factor by MicroRNA-214 delivery in exosomes from mouse or human hepatic stellate cells. Hepatology 59, 1118-1129, doi:10.1002/hep.26768 (2014).
9 Calvente, C. J. et al. Neutrophils contribute to spontaneous resolution of liver inflammation and fibrosis via microRNA-223. J Clin Invest 129, 4091-4109, doi:10.1172/JCI122258 (2019).
10 He, Y. et al. MicroRNA-223 Ameliorates Nonalcoholic Steatohepatitis and Cancer by Targeting Multiple Inflammatory and Oncogenic Genes in Hepatocytes. Hepatology 70, 1150-1167, doi:10.1002/hep.30645 (2019).
11 Sveger, T. The natural history of liver disease in alpha 1-antitrypsin deficient children. Acta Paediatr Scand 77, 847-851 (1988).
12 Piitulainen, E., Carlson, J., Ohlsson, K. & Sveger, T. Alpha1-antitrypsin deficiency in 26-year-old subjects: lung, liver, and protease/protease inhibitor studies. Chest 128, 2076-2081, doi:128/4/2076 [pii] 10.1378/chest.128.4.2076 (2005).
13 Eriksson, S., Carlson, J. & Velez, R. Risk of cirrhosis and primary liver cancer in alpha 1-antitrypsin deficiency. The New England journal of medicine 314, 736-739, doi:10.1056/NEJM198603203141202 (1986).
14 Szabo, G. & Bala, S. MicroRNAs in liver disease. Nat Rev Gastroenterol Hepato/ 10, 542-552, doi:10.1038/nrgastro.2013.87 (2013).
15 Carlson, J. A. et al. Accumulation of PiZ alpha 1-antitrypsin causes liver damage in transgenic mice. The Journal of clinical investigation 83, 1183-1190 (1989).
16 Concepcion, C. P. et al. Intact p53-dependent responses in miR-34-deficient mice. PLOS genetics 8, e1002797, doi:10.1371/journal.pgen.1002797 (2012).
17 Kim, Y. O., Popov, Y. & Schuppan, D. Optimized Mouse Models for Liver Fibrosis. Methods in molecular biology 1559, 279-296, doi:10.1007/978-1-4939-6786-5_19 (2017).
18 Doria, M., Ferrara, A. & Auricchio, A. AAV2/8 vectors purified from culture medium with a simple and rapid protocol transduce murine liver, muscle, and retina efficiently. Human gene therapy methods 24, 392-398, doi:10.1089/hgtb.2013.155 (2013).
19 Ishak, K. et al. Histological grading and staging of chronic hepatitis. Journal of hepatology 22, 696-699, doi:10.1016/0168-8278(95)80226-6 (1995).
20 Piccolo, P. et al. Up-regulation of miR-34b/c by JNK and FOXO3 protects from liver fibrosis. Proceedings of the National Academy of Sciences 118, e2025242118, doi:10.1073/pnas.2025242118 (2021).
21 Dobin, A. et al. STAR: ultrafast universal RNA-seq aligner. Bioinformatics 29, 15-21, doi:10.1093/bioinformatics/bts635 (2013).
22 Lee, C. M. et al. UCSC Genome Browser enters 20th year. Nucleic Acids Res 48, D756-D761, doi:10.1093/nar/gkz1012 (2020).
23 Anders, S., Pyl, P. T. & Huber, W. HTSeq—a Python framework to work with high-throughput sequencing data. Bioinformatics 31, 166-169, doi:10.1093/bioinformatics/btu638 (2015).
24 Love, M. I., Huber, W. & Anders, S. Moderated estimation of fold change and dispersion for RNA-seq data with DESeq2. Genome Biol 15, 550, doi:10.1186/s13059-014-0550-8 (2014).
25 Subramanian, A. et al. Gene set enrichment analysis: a knowledge-based approach for interpreting genome-wide expression profiles. Proc Natl Acad Sci U S A 102, 15545-15550, doi:10.1073/pnas.0506580102 (2005).
26 Zhang, D. Y. et al. A hepatic stellate cell gene expression signature associated with outcomes in hepatitis C cirrhosis and hepatocellular carcinoma after curative resection. Gut 65, 1754-1764, doi:10.1136/gutjnl-2015-309655 (2016).
27 Dennis, G., Jr. et al. DAVID: Database for Annotation, Visualization, and Integrated Discovery. Genome biology 4, P3 (2003).
28 Reczko, M., Maragkakis, M., Alexiou, P., Grosse, I. & Hatzigeorgiou, A. G. Functional microRNA targets in protein coding sequences. Bioinformatics 28, 771-776, doi:10.1093/bioinformatics/bts043 (2012).
29 Paraskevopoulou, M. D. et al. DIANA-microT web server v5.0: service integration into miRNA functional analysis workflows. Nucleic acids research 41, W169-173, doi:10.1093/nar/gkt393 (2013).
30 Garofalo, M. et al. MiR-34a/c-Dependent PDGFR-alpha/beta Downregulation Inhibits Tumorigenesis and Enhances TRAIL-Induced Apoptosis in Lung Cancer. PloS one 8, e67581, doi:10.1371/journal.pone.0067581 (2013).
31 Song, L. J. et al. The Differential and Dynamic Progression of Hepatic Inflammation and Immune Responses During Liver Fibrosis Induced by Schistosoma japonicum or Carbon Tetrachloride in Mice. Frontiers in immunology 11, 570524, doi:10.3389/fimmu.2020.570524 (2020).
32 He, L. et al. A microRNA component of the p53 tumour suppressor network. Nature 447, 1130-1134, doi:10.1038/nature05939 (2007).
33 Arteel, G. E. & Naba, A. The liver matrisome - looking beyond collagens. JHEP reports: innovation in hepatology 2, 100115, doi:10.1016/j.jhepr.2020.100115 (2020).
34 Balakrishnan, I. et al. Genome-wide analysis of miRNA-mRNA interactions in marrow stromal cells. Stem cells 32, 662-673, doi:10.1002/stem.1531 (2014).
35 Salguero Palacios, R. et al. Activation of hepatic stellate cells is associated with cytokine expression in thioacetamide-induced hepatic fibrosis in mice. Laboratory Investigation 88, 1192-1203, doi:10.1038/labinvest.2008.91 (2008).
36 Harris, R., Harman, D. J., Card, T. R., Aithal, G. P. & Guha, I. N. Prevalence of clinically significant liver disease within the general population, as defined by non-invasive markers of liver fibrosis: a systematic review. The lancet. Gastroenterology & hepatology 2, 288-297, doi:10.1016/S2468-1253(16)30205-9 (2017).
37 Ramachandran, P. & Henderson, N. C. Antifibrotics in chronic liver disease: tractable targets and translational challenges. The lancet. Gastroenterology & hepatology 1, 328-340, doi:10.1016/S2468-1253(16)30110-8 (2016).
38 Nevens, F. et al. A Placebo-Controlled Trial of Obeticholic Acid in Primary Biliary Cholangitis. The New England journal of medicine 375, 631-643, doi:10.1056/NEJMoa1509840 (2016).
39 Kowdley, K. V. et al. A randomized trial of obeticholic acid monotherapy in patients with primary biliary cholangitis. Hepatology 67, 1890-1902, doi:10.1002/hep.29569 (2018). 40 Hong, J. S. et al. MicroRNA signatures associated with thioacetamide-induced liver fibrosis in mice. Bioscience, biotechnology, and biochemistry 81, 1348-1355, doi:10.1080/09168451.2017.1308242 (2017).
41 Hyun, J. et al. MicroRNA-378 limits activation of hepatic stellate cells and liver fibrosis by suppressing Gli3 expression. Nature communications 7, 10993, doi:10.1038/ncomms10993 (2016).
42 Li, W. Q. et al. The rno-miR-34 family is upregulated and targets ACSL1 in dimethylnitrosamine-induced hepatic fibrosis in rats. The FEBS journal 278, 1522-1532, doi:10.1111/j.1742-4658.2011.08075.x (2011).
43 Murakami, Y. et al. The progression of liver fibrosis is related with overexpression of the miR-199 and 200 families. PloS one 6, e16081, doi:10.1371/journal.pone.0016081 (2011).
44 Li, X. et al. microRNA-34a and microRNA-34c promote the activation of human hepatic stellate cells by targeting peroxisome proliferator-activated receptor gamma. Molecular medicine reports 11, 1017-1024, doi:10.3892/mmr.2014.2846 (2015).
45 Tian, X. F., Ji, F. J., Zang, H. L. & Cao, H. Activation of the miR-34a/SIRT1/p53 Signaling Pathway Contributes to the Progress of Liver Fibrosis via Inducing Apoptosis in Hepatocytes but Not in HSCs. PloS one 11, e0158657, doi:10.1371/journal.pone.0158657 (2016).
46 Wan, Y. et al. Regulation of Cellular Senescence by miR-34a in Alcoholic Liver Injury. The American journal of pathology 187, 2788-2798, doi:10.1016/j.ajpath.2017.08.027 (2017).
47 Yan, G. et al. MicroRNA-34a Promotes Hepatic Stellate Cell Activation via Targeting ACSL1. Medical science monitor: international medical journal of experimental and clinical research 21, 3008-3015, doi:10.12659/MSM.894000 (2015).
48 Zhang, F., Lu, Y. & Zheng, S. Peroxisome proliferator-activated receptor-gamma cross-regulation of signaling events implicated in liver fibrogenesis. Cellular signalling 24, 596-605, doi:10.1016/j.cellsig.2011.11.008 (2012).
49 Chen, L. et al. Therapeutic effects of serum extracellular vesicles in liver fibrosis. Journal of extracellular vesicles 7, 1461505, doi:10.1080/20013078.2018.1461505 (2018).
50 Rupaimoole, R. & Slack, F. J. MicroRNA therapeutics: towards a new era for the management of cancer and other diseases. Nature reviews. Drug discovery 16, 203-222, doi:10.1038/nrd.2016.246 (2017).
51 Chakraborty, C., Sharma, A. R., Sharma, G., Doss, C. G. P. & Lee, S. S. Therapeutic miRNA and SIRNA: Moving from Bench to Clinic as Next Generation Medicine. Molecular therapy. Nucleic acids 8, 132-143, doi:10.1016/j.omtn.2017.06.005 (2017).
52 Beg, M. S. et al. Phase I study of MRX34, a liposomal miR-34a mimic, administered twice weekly in patients with advanced solid tumors. Investigational new drugs 35, 180-188, doi:10.1007/s10637-016-0407-y (2017).
53 Winkle, M., El-Daly, S. M., Fabbri, M. & Calin, G. A. Noncoding RNA therapeutics - challenges and potential solutions. Nature reviews. Drug discovery, doi:10.1038/s41573-021-00219-z (2021).
54 Carlson, J. A. et al. Multiple tissues express alpha 1-antitrypsin in transgenic mice and man. The Journal of clinical investigation 82, 26-36, doi:10.1172/JCI113580 (1988).
55 Smit, J. J. et al. Homozygous disruption of the murine mdr2 P-glycoprotein gene leads to a complete absence of phospholipid from bile and to liver disease. Cell 75, 451-462 (1993).
56 Mueller, C. et al. Sustained miRNA-mediated knockdown of mutant AAT with simultaneous augmentation of wild-type AAT has minimal effect on global liver miRNA profiles. Mol Ther 20, 590-600, doi:10.1038/mt.2011.292 (2012).
57 Gao, G. P. et al. Novel adeno-associated viruses from rhesus monkeys as vectors for human gene therapy. Proceedings of the National Academy of Sciences of the United States of America 99, 11854-11859, doi:10.1073/pnas.182412299 (2002).
58 Tag, C. G. et al. Bile duct ligation in mice: induction of inflammatory liver injury and fibrosis by obstructive cholestasis. Journal of visualized experiments: JoVE, doi:10.3791/52438 (2015).
59 Attanasio, S. et al. CHOP and c-JUN upregulate the mutant Z alphα-1 antitrypsin, exacerbating its aggregation and liver proteotoxicity. Journal of Biological Chemistry, doi:10.1074/jbc.RA120.014307 (2020). 60 Ferriero, R. et al. Pyruvate dehydrogenase complex and lactate dehydrogenase are targets for therapy of acute liver failure. J Hepatol 69, 325-335, doi:10.1016/j.jhep.2018.03.016 (2018).
61 Kent, W. J. et al. The human genome browser at UCSC. Genome Res. 12, 996-1006, doi:10.1101/gr.229102 (2002).
62 Robinson, M. D., Mccarthy, D. J. & Smyth, G. K. edgeR: a Bioconductor package for differential expression analysis of digital gene expression data. Bioinformatics 26, 139-140, doi:10.1093/bioinformatics/btp616 (2010).
63 Webb, A. E., Kundaje, A. & Brunet, A. Characterization of the direct targets of FOXO transcription factors throughout evolution. Aging cell 15, 673-685, doi:10.1111/acel.12479 (2016).
64 van Koppen, A. et al. Uncovering a Predictive Molecular Signature for the Onset of NASH-Related Fibrosis in a Translational NASH Mouse Model. Cellular and molecular gastroenterology and hepatology 5, 83-98 e10, doi:10.1016/j.jcmgh.2017.10.001 (2018).
65 Lau-Corona, D., Bae, W. K., Hennighausen, L. & Waxman, D. J. Sex-biased genetic programs in liver metabolism and liver fibrosis are controlled by EZH1 and EZH2. PLOS genetics 16, e1008796, doi:10.1371/journal.pgen.1008796 (2020).
66 Griffiths-Jones, S., Grocock, R. J., van Dongen, S., Bateman, A. & Enright, A. J. miRBase: microRNA sequences, targets and gene nomenclature. Nucleic acids research 34, D140-144, doi:10.1093/nar/gkj112 (2006).
67 Mccarthy, D. J., Chen, Y. & Smyth, G. K. Differential expression analysis of multifactor RNA-Seq experiments with respect to biological variation. Nucleic acids research 40, 4288-4297, doi:10.1093/nar/gks042 (2012).
68 Team, R. C. (R Foundation for Statistical Computing, Vienna (Austria), 2015).
69 Kirschner, M. B. et al. Haemolysis during sample preparation alters microRNA content of plasma. PloS one 6, e24145, doi:10.1371/journal.pone.0024145 (2011).
70 Geller, S. A., Nichols, W. S., Dycaico, M. J., Felts, K. A. & Sorge, J. A. Histopathology of alpha 1-antitrypsin liver disease in a transgenic mouse model. Hepatology 12, 40-47 (1990).
71 Kress, T. R. et al. The MK5/PRAK kinase and Myc form a negative feedback loop that is disrupted during colorectal tumorigenesis. Molecular cell 41, 445-457, doi:10.1016/j.molcel.2011.01.023 (2011).
72 Masui, K. et al. mTOR complex 2 controls glycolytic metabolism in glioblastoma through FoxO acetylation and upregulation of c-Myc. Cell metabolism 18, 726-739, doi:10.1016/j.cmet.2013.09.013 (2013).
⁷³ Tikhanovich, I. et al. Regulation of FOXO3 by phosphorylation and methylation in hepatitis C virus infection and alcohol exposure. Hepatology 59, 58-70, doi:10.1002/hep.26618 (2014).
74 Pastore, N. et al. Activation of the c-Jun N-terminal kinase pathway aggravates proteotoxicity of hepatic mutant Z alpha1-antitrypsin. Hepatology 65, 1865-1874, doi:10.1002/hep.29035 (2017).
75 Borkham-Kamphorst, E. & Weiskirchen, R. The PDGF system and its antagonists in liver fibrosis. Cytokine & growth factor reviews 28, 53-61, doi:10.1016/j.cytogfr.2015.10.002 (2016).
76 Kocabayoglu, P. et al. beta-PDGF receptor expressed by hepatic stellate cells regulates fibrosis in murine liver injury, but not carcinogenesis. Journal of hepatology 63, 141-147, doi:10.1016/j.jhep.2015.01.036 (2015).
77 Kikuchi, A. et al. Platelet-Derived Growth Factor Receptor alpha Contributes to Human Hepatic Stellate Cell Proliferation and Migration. The American journal of pathology 187, 2273-2287, doi:10.1016/j.ajpath.2017.06.009 (2017).
78 Campbell, J. S. et al. Platelet-derived growth factor C induces liver fibrosis, steatosis, and hepatocellular carcinoma. Proceedings of the National Academy of Sciences of the United States of America 102, 3389-3394, doi:10.1073/pnas.0409722102 (2005).
79 Mauad, T. H. et al. Mice with homozygous disruption of the mdr2 P-glycoprotein gene. A novel animal model for studies of nonsuppurative inflammatory cholangitis and hepatocarcinogenesis. The American journal of pathology 145, 1237-1245 (1994).
80 Kluwe, J. et al. Modulation of hepatic fibrosis by c-Jun-N-terminal kinase inhibition. Gastroenterology 138, 347-359, doi:10.1053/j.gastro.2009.09.015 (2010).
81 Kwak, B. J. et al. The Role of Phospho-c-Jun N-Terminal Kinase Expression on hepatocyte Necrosis and Autophagy in the Cholestatic Liver. The Journal of surgical research 241, 254-263, doi:10.1016/j.jss.2019.03.034 (2019).
82 Seki, E., Brenner, D. A. & Karin, M. A liver full of JNK: signaling in regulation of cell function and disease pathogenesis, and clinical approaches. Gastroenterology 143, 307-320, doi:10.1053/j.gastro.2012.06.004 (2012).
83 Ho, K. K. et al. Phosphorylation of FOXO3a on Ser-7 by p38 promotes its nuclear localization in response to doxorubicin. The Journal of biological chemistry 287, 1545-1555, doi:10.1074/jbc.M111.284224 (2012).
84 Lindblad, D., Blomenkamp, K. & Teckman, J. Alpha-1-antitrypsin mutant Z protein content in individual hepatocytes correlates with cell death in a mouse model. Hepatology 46, 1228-1235, doi:10.1002/hep.21822 (2007).
85 Singh, A. K. et al. Global microRNA expression profiling in the liver biopsies of hepatitis B virus-infected patients suggests specific microRNA signatures for viral persistence and hepatocellular injury. Hepatology 67, 1695-1709, doi:10.1002/hep.29690 (2018).
86 Brunt, E. M. et al. Hepatic progenitor cell proliferation and liver injury in alpha-1-antitrypsin deficiency. Journal of pediatric gastroenterology and nutrition 51, 626-630, doi:10.1097/MPG.0b013e3181e7ff55 (2010).
87 Lim, B. J. et al. Selective deletion of hepatocyte platelet-derived growth factor receptor a and development of liver fibrosis in mice. Cell Communication and Signaling 16, 93, doi:10.1186/s12964-018-0306-2 (2018).
88 Al-Tamari, H. M. et al. Fox03 an important player in fibrogenesis and therapeutic target for idiopathic pulmonary fibrosis. EMBO Mol Med 10, 276-293, doi:10.15252/emmm.201606261 (2018).
89 Clark, V. C. et al. Clinical and Histologic Features of Adults with Alphα-1 Antitrypsin Deficiency in a Non-Cirrhotic Cohort. Journal of hepatology, doi:10.1016/j.jhep.2018.08.005 (2018).
90 Hamesch, K. et al. Liver Fibrosis and Metabolic Alterations in Adults with Alpha1 Antitrypsin Deficiency Caused by the Pi*ZZ Mutation. Gastroenterology, doi:10.1053/j.gastro.2019.05.013 (2019).
91 Xu, Y. et al. A potentially functional polymorphism in the promoter region of miR-34b/c is associated with an increased risk for primary hepatocellular carcinoma. Int J Cancer 128, 412-417, doi:10.1002/ijc.25342 (2011).
92 Son, M. S. et al. Promoter polymorphisms of pri-miR-34b/c are associated with hepatocellular carcinoma. Gene 524, 156-160, doi:10.1016/j.gene.2013.04.042 (2013).
93 Strnad, P. et al. Heterozygous carriage of the alpha1-antitrypsin Pi*Z variant increases the risk to develop liver cirrhosis. Gut, doi:10.1136/gutjnl-2018-316228 (2018).
94 Henderson, N. C., Rieder, F. & Wynn, T. A. Fibrosis: from mechanisms to medicines. Nature 587, 555-566, doi:10.1038/s41586-020-2938-9 (2020).
95 Trautwein, C., Friedman, S. L., Schuppan, D. & Pinzani, M. Hepatic fibrosis: Concept to treatment. J Hepatol 62, S15-24, doi:10.1016/j.jhep.2015.02.039 (2015).

1. A method for the treatment and/or prevention of fibrosis and of diseases associated with fibrosis, comprising administering an effective amount of an agent selected from the group consisting of:
a combination of:
miR-34b or a precursor or a mimic or a functional derivative thereof and
miR-34c or a precursor or a mimic or a functional derivative thereof; or
miR-34b or a precursor or a mimic or a functional derivative thereof or
miR-34c or a precursor or a mimic or a functional derivative thereof:
to a patient in need thereof.

2. The method according to claim 1 wherein it the agent comprises a double-stranded RNA molecule 22 to 24 basepairs in length comprising:
a) an active strand comprising miR-34b or miR-34c and b) a passenger strand comprising a sequence that is at least 60%, 70%, 80%, 90% or 100% complementary to the active strand, optionally said RNA molecule being blunt-ended.

3. The method according to claim 1, whereinthe miR-34b comprises the SEQ ID NO: 3 or 1, and/or miR-34c comprises the SEQ ID NO: 11 or 9.

4. The method according to claim 1, wherein said agent is provided within a delivery vehicle, optionally wherein the delivery vehicle is selected from the group consisting of a vector, and a delivery vehicle selected from nanoparticles, microparticles, liposomes or other biological or synthetic vesicle or material including lipid nanoparticles, polymer-based nanoparticles, polymer-lipid hybrid nanoparticles, microparticles, microspheres, liposomes, colloidal gold particles, graphene composites, cholesterol conjugates, cyclodextran complexes, polyethylenimine e polymers, lipopolysaccharides, polypeptides, polysaccharides, lipopolysaccharides, collagen, pegylation of viral vehicles.

5. A method for the treatment and/or prevention of fibrosis and of diseases associated with fibrosis comprising administering a nucleic acid coding for an agent of claim 1 to a patient in need thereof.

6. (canceled)

7. The method of claim 13, wherein a host cell transformed with thea vector is administered.

8. A The method of claim 5, wherein a recombinant adeno-associated virus (rAAV) particle comprises a nucleic acid encoding miR-34b and/or miR-34c or a precursor or a mimic or a functional derivative thereof.

9. (canceled)

10. A method for the diagnosis of fibrosis and/or of diseases associated with fibrosis and/or for determining the activity, the stage, or the severity of fibrosis and/or of diseases associated with fibrosis in a subject, and/or for the classification of a subject as a receiver or non receiver of a treatment for fibrosis and/or of diseases associated with fibrosis, and/or for the evaluation of the efficacy of a medical treatment, and/or for the determination of the progression or the regression of the disease in fibrosis patients or in diseases associated with fibrosis patients, and/or for the classification of a patient as a potential responder or non responder to a medical treatment, and/or for the prediction of disease outcome for a patient comprising determining the level of miR-34b and/or miR34c in a sample obtained from a subject and comparing it with a proper control.

11. A kit for the diagnosis of fibrosis and/or of diseases associated with fibrosis and/or for determining the activity, the stage, or the severity of fibrosis and/or of diseases associated with fibrosis in a subject, and/or for the classification of a subject as a receiver or non receiver of a treatment for fibrosis and/or for diseases associated with fibrosis, and/or for the evaluation of the efficacy of a medical treatment, and/or for the determination of the progression or the regression of the disease in fibrosis patients or in diseases associated with fibrosis patients, and/or for the classification of a patient as a potential responder or non responder to a medical treatment, and/or for the prediction of disease outcome comprising primers and/or probes specific for miR-34b and miR-34c, or for miR-34b or for miR-34c.

12. The method of claim 1 wherein the fibrosis is a fibrosis of liver, lungs, kidneys, skin, joints and/or the disease associated with fibrosis is an acquired or genetic diseases selected from the group consisting of: Cholestatic liver diseases, such as Primary Sclerosing Cholangitis, Primary Biliary Cholangitis, Primary Familiar Intrahepatic Cholestasis, Non-alcoholic fatty liver disease (NAFLD)/Non-alcoholic steatohepatitis (NASH) preferably with advanced fibrosis, Viral hepatitis, Genetic diseases affecting liver, such as Wilson disease, Primary Familiar Intrahepatic Cholestasis, A1AT deficiency, Haemochromatosis, Congenital Hepatic Fibrosis.

13. The method of claim 4, wherein the vector-comprises a coding sequence for and/or expresses an agent selected from the group consisting of:
miR-34b or a precursor or a mimic or a functional derivative thereof; and
miR-34c or a precursor or a mimic or a functional derivative thereof.

14. The method of claim 4, wherein the vector is a viral or non-viral vector, and the viral vector is optionally selected from adeno-associated virus (AAV) vectors, lentivirus vectors, adenoviral vector, retroviral vectors, alphaviral vectors, vaccinia virus vectors, herpes simplex virus (HSV) vectors, rabies virus vectors, and Sindbis virus vectors.
